(19) United States
(12) Patent Application Publication
(10) Pub. No.: US 2026/0201315 A1
(43) Pub. Date: Jul. 16, 2026

(54) INCREASED NITROGEN FIXATION USING BACTERIA WITH IMPROVED AMMONIA SECRETION

(71) Applicants: Bayer CropScience LLC, St. Louis, MO (US); Ginkgo Bioworks, Inc., Boston, MA (US)

(72) Inventor: Sue Zanne TAN, Cambridge, MA (US)

(21) Appl. No.: 19/129,826

(22) PCT Filed: Nov. 17, 2023
(86) PCT No.: PCT/US2023/080240

§ 371 Date: May 14, 2025

Related U.S. Application Data
(60) Provisional application No. 63/384,373, filed on Nov. 18, 2022.

(51) Int. Cl. C12N 1/20; C05F 11/08; C07K 14/195; C12N 15/11; C12N 15/74; C12R 1/01

(52) U.S. Cl. CPC C12N 1/20; C05F 11/08; C07K 14/195; C12N 15/11; C12N 15/74; C12N 2830/34; C12R 2001/01

(57) ABSTRACT

Herbaspirillum bacterial strains are engineered to express or overexpress one or more proteins and/or attenuate expression of or delete one or more genes involved in nitrogen fixation and its regulation. This enables the diazotrophic bacteria to secrete more ammonia. The engineered bacteria arc introduced to agricultural plants, or seeds for growing such, forming a symbiotic relationship that results in increased nitrogen availability to the plant and improved plant agronomic traits.

### CROSS-REFERENCE TO RELATED APPLICATIONS

This application claims priority to U.S. Provisional Patent Application No. 63/384,373, filed Nov. 18, 2022, the contents of which are incorporated herein by reference in their entirety.

### REFERENCE TO SEQUENCE LISTING SUBMITTED ELECTRONICALLY

The official copy of the sequence listing is submitted electronically via EFS-Web as an XML-formatted sequence listing with a file named “BCS229003_WO_SequenceListing.xml” created on Nov. 15, 2023, and having a size of 151 kilobytes, and is filed concurrently with the specification. The sequence listing contained in this XML-formatted document is part of the specification and is incorporated herein by reference in its entirety.

### FIELD OF THE DISCLOSURE

The present disclosure relates to Herbaspirillum microbes engineered to increase nitrogen fixation and their association with an agricultural plant to provide increased nitrogen availability to the plant and improved agronomic traits for the plant. Compositions including the Herbaspirillum microbes and/or plants associated or grown from the microbes and methods of preparing and using the compositions are also disclosed.

### BACKGROUND OF THE DISCLOSURE

Plant growth requires nitrogen. Although nitrogen makes up 80% of the atmosphere in the form of dinitrogen gas (N₂), plants cannot utilize N₂ directly. Plants obtain nitrogen in the form of ammonium (NH₄⁺) and nitrates from the soil by absorption through their roots. Nitrogen-based (N-based) fertilizers have been developed to address the limiting nature of nitrogen for plant growth, but inefficient use of the N-based fertilizers by plants have contributed to contamination of soils and water resources and pose a hazard to human health.

Some microorganisms are able to convert atmospheric N₂ to reduced forms of nitrogen such as ammonia and amino acids, which can be used by plants. Microorganisms that can perform this biological nitrogen fixation are called diazotrophs. Diazotrophic microorganisms have nitrogenase, an enzyme complex that fixes N₂ to NH₃. Several associations between diazotrophs and their host plants are known that typically involve the formation of root nodules on the plant. The associations can be endophytic, meaning the microorganism colonizes the plant tissue and in some instances they colonize without causing any apparent symptoms of disease. For example, gram-negative rhizobia interact with legume and non-legume plants, gram positive actinomycetes associate with actinorhizal plants, and some cyanobacteria can form symbiotic associations with a variety of plants. Herbaspirillum is a gram-negative endophytic diazotroph that associates with cereals. In these diazotroph-host plant interactions, the microorganism provides fixed nitrogen to the plant and in turn, the plant provides reduced carbon and possibly other nutrients to the diazotroph.

Nitrogen fixing microorganisms thus hold promise as a more environmentally friendly alternative to chemical fertilizers and could offer a route to enhance plant growth, improve sustainability of agriculture, and reduce damage to the environment and human health. With the advent of technologies that allow sequencing and study of genomes, it is now possible to exploit knowledge of the genomes of diazotrophs to engineer them for more efficient nitrogen fixation.

### SUMMARY OF THE DISCLOSURE

The present disclosure provides an Herbaspirillum seropedicae (Hs) microbe genetically modified to increase nitrogen fixation compared to a non-genetically modified Hs microbe. Nitrogen fixation is increased in Hs by strategies including: increasing conversion of nitrogen to ammonia by increasing nitrogenase activity, preventing re-assimilation of secreted ammonia into the cell, reducing Hs incorporation of fixed ammonia into amino acids/biomass, and modifying expression or activity of nitrogen regulatory proteins. The genetically modified Hs microbe is associated with agricultural plants, or the seeds for growing such agricultural plants, forming a commensal relationship that results in increased nitrogen availability to the plant and improved plant agronomic traits.

In some embodiments, the genetically modified Hs microbe includes: (a) a heterologous NifA gene encoding transcriptional activator NifA; and (b) downregulation of expression of endogenous AmtB gene encoding ammonium transporter AmtB. In particular embodiments, downregulation of expression of the endogenous AmtB gene includes replacement of the endogenous AmtB gene with an expression cassette including the heterologous NifA gene such that expression of AmtB is reduced or eliminated. In particular embodiments, the NifA expression cassette includes heterologous NifA gene operably linked to a constitutive promoter. In particular embodiments, the constitutive promoter is P65. In particular embodiments, the constitutive promoter is CP25. In particular embodiments, the constitutive promoter is CP32. In particular embodiments, the NifA expression cassette is integrated at a neutral site in the Hs microbe genome. In other embodiments the heterologous NifA gene is Hs NifA.

In some embodiments, the genetically modified Hs microbe includes downregulation of expression of the endogenous ginK gene encoding PII-like nitrogen regulatory protein. In particular embodiments, downregulation of expression of the endogenous ginK gene includes a deletion of the endogenous ginK gene.

In some embodiments, the genetically modified Hs microbe includes endogenous nifA gene operably linked to a heterologous constitutive promoter. In particular embodiments, the constitutive promoter is P65. In particular embodiments, the constitutive promoter is CP25. In particular embodiments, the constitutive promoter is CP32.

In particular embodiments, the genetically modified Hs microbe includes: (a) downregulated endogenous ginK gene encoding P11-like nitrogen regulatory protein; (b) a heterologous nifA gene encoding transcriptional activator NifA operably linked to a first heterologous constitutive promoter; (c) downregulated endogenous amtB gene encoding ammonium transporter AmtB; and (d) endogenous nifA gene operably linked to a second heterologous constitutive promoter, wherein downregulation of expression of the endogenous amtB gene includes replacement of the endogenous amtB gene with the heterologous nifA gene operably linked to the first constitutive promoter such that expression of AmtB is reduced or eliminated, and wherein the second heterologous constitutive promoter replaces the endogenous nifA promoter. In particular embodiments, downregulated endogenous glnK gene includes a deletion of the endogenous glnK gene.

In some embodiments, the genetically modified Hs microbe includes downregulated endogenous ntrC gene encoding nitrogen regulatory protein NtrC. In some embodiments, the genetically modified Hs microbe includes a deletion of the endogenous ntrC gene encoding nitrogen regulatory protein NtrC and deletion of endogenous AmtB gene encoding ammonium transporter AmtB.

In some embodiments, the genetically modified Hs microbe includes downregulated endogenous nifH gene encoding dinitrogenase reductase nifH. In some embodiments, the genetically modified Hs microbe includes a deletion of the endogenous nifH gene encoding dinitrogenase reductase nifH.

In particular embodiments, the genetically modified Hs microbe includes endogenous glnA gene encoding glutamine synthetase (GS) attenuated in expression and/or activity as compared to an Hs microbe that is not genetically modified. In particular embodiments, the attenuated expression of the GS includes expression of the endogenous glnA gene operably linked to a heterologous promoter. In particular embodiments, the heterologous promoter drives weaker expression of glnA as compared to endogenous Hs glnA gene promoter. In particular embodiments, the attenuated expression of the GS includes expression of the endogenous glnA gene operably linked to a ribosomal binding site (RBS) modified to decrease translation efficiency of the endogenous glnA gene as compared to the endogenous glnA gene operably linked to the corresponding endogenous unmodified RBS. In particular embodiments, the attenuated expression of the GS includes expression of the endogenous glnA gene operably linked to a degradation tag.

In particular embodiments, the genetically modified Hs microbe includes an amino (N)-terminal truncation of the endogenous glnE gene encoding an adenylyl transferase to attenuate activity of GS. In particular embodiments, the N-terminal truncation is from amino acid residue 1 to 260 of Hs glnE gene.

In particular embodiments, the genetically modified Hs microbe does not include a selectable marker or a counter-selection marker.

In particular embodiments, nitrogenase activity of a genetically modified Hs microbe is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to nitrogenase activity of a control Hs microbe as measured by an acetylene reduction assay, an ¹⁵N₂ fixing assay, an ¹⁵N dilution assay, and/or an ammonia biosensor assay. In particular embodiments, ammonia secretion from a genetically modified Hs microbe is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to ammonia secretion of a control Hs microbe as measured by an ammonia biosensor assay. In particular embodiments, a control Hs microbe includes a non-genetically modified Hs microbe. In particular embodiments, a control Hs microbe includes an Hs microbe with increased nitrogen fixation as compared to a non-genetically modified Hs microbe. In particular embodiments, the ammonia biosensor assay includes an Hs microbe including a deletion of endogenous nifH gene and a detectable reporter expression cassette.

The disclosure also provides a formulation including a genetically modified Hs microbe and a carrier. In particular embodiments, the formulation includes a stabilizer, a surfactant, an adherent, a fungicide, a nematicide, an insecticide, an herbicide, a virucide, a nutrient, or any combination thereof. In particular embodiments, the formulation includes a seed coating.

The disclosure also provides a composition including a plant or plant part and a genetically modified Hs microbe disclosed herein. In particular embodiments, the genetically modified Hs microbe is included in a formulation. In particular embodiments, the plant part is a seed and the formulation includes a seed coating. In particular embodiments, the composition further includes a medium that promotes plant growth.

Particular embodiments provide for a method for preparing a composition, including contacting the surface of a plant or plant part with a formulation including a genetically modified Hs microbe disclosed herein to produce an inoculated plant or plant part, wherein the genetically modified Hs microbe is present in the formulation in an amount capable of improving an agronomic trait of the plant grown from the inoculated plant or plant part.

Particular embodiments provide for a method of inoculating a plant or plant part, including contacting a plant or plant part with a formulation including a genetically modified Hs microbe disclosed herein. In particular embodiments, the method further includes growing the inoculated plant or plant part. Particular embodiments provide for a plant or part thereof grown from the inoculated plant or plant part.

Particular embodiments provide for a method of improving an agronomic trait in a plant, including growing a plant from a plant or plant part that has been contacted with a formulation including a genetically modified Hs microbe disclosed herein. In particular embodiments, chlorophyll content of a genetically modified Hs microbe-associated plant is increased 3 to 20% as compared to chlorophyll content of a reference agricultural plant. In particular embodiments, fresh shoot weight of a genetically modified Hs microbe-associated plant is increased 3 to 20% as compared to fresh shoot weight of a reference agricultural plant. In particular embodiments, shoot dry weight of a genetically modified Hs microbe-associated plant is increased 3 to 20% as compared to shoot dry weight of a reference agricultural plant.

Particular embodiments provide for a method of reducing nitrogen fertilizer application, including growing a plant from a plant or plant part that has been contacted with a formulation including a genetically modified Hs microbe disclosed herein. In particular embodiments, the application of nitrogen to the plant is reduced as compared to application of nitrogen to a control plant grown from a plant or plant part that has not been contacted with the formulation. In particular embodiments, the reduction in the application of nitrogen is measured as N replacement, and wherein the N replacement per application is at least 5 ppm of N, at least 6 ppm of N, at least 7 ppm of N, at least 8 ppm of N, at least 9 ppm of N, at least 10 ppm of N, at least 11 ppm of N, at least 12 ppm of N, at least 13 ppm of N, at least 14 ppm of N, at least 15 ppm of N, at least 16 ppm of N, or greater.

In some embodiments, the plant or plant part includes: maize, wheat, soybean, barley, millet, rice, turfgrass, cotton, canola, rapeseed, alfalfa, tomato, sugarbeet, oats, rye, sorghum, almond, walnut, apple, peanut, strawberry, lettuce, orange, potato, banana, sugarcane, potato, cassava, mango, guava, palm, onions, olives, peppers, tea, yams, cacao, sunflower, asparagus, carrot, coconut, lemon, lime, watermelon, cabbage, cucumber, and grape.

Particular embodiments provide for a genetic construct. In some embodiments, the genetic construct includes 5′ to 3′: (a) an upstream (US) homology arm including sequence homologous to sequence 5′ of the start codon of an endogenous Hs glnK coding sequence; and (b) a downstream (DS) homology arm including sequence homologous to sequence 3′ of the stop codon of the endogenous Hs glnK coding sequence. In some embodiments, the genetic construct includes 5′ to 3′: (a) a US homology arm including sequence homologous to sequence 5′ of the start codon of an endogenous Hs amtB coding sequence; (b) a P65 promoter; (c) a nifA coding sequence; and (d) a DS homology arm including sequence homologous to sequence 3′ of the stop codon of the endogenous Hs amtB coding sequence. In some embodiments, the genetic construct includes 5′ to 3′: (a) a US homology arm including sequence homologous to sequence 5′ of an endogenous Hs nifA promoter; (b) a CP25 promoter; and (c) a DS homology arm including sequence homologous to sequence 3′ of the endogenous Hs nifA promoter. Particular embodiments provide for a cell including a genetic construct disclosed herein.

### BRIEF DESCRIPTION OF THE DRAWINGS

FIG. 1. Nitrogen fixation pathway in Herbaspirillum seropedicae (Hs) and the delivery of fixed nitrogen to plants: (1) Nitrogenase enzyme complex converts nitrogen to ammonia; (2) uptake of secreted nitrogen into the Hs cell is hypothesized to involve an AmtB ammonium transporter; (3) incorporation of ammonia into amino acids is catalyzed by glutamine synthetase, which converts ammonia and glutamate into glutamine.

FIG. 2. A schematic of a nif cluster in Hs.

FIGS. 3A-3I. Genetic constructs of the disclosure in a suicide vector backbone. The genetic constructs are described in Example 1.

FIG. 4. A schematic showing a transformation process of a genetic construct (e.g., ΔamtB::P65:nifA:TrrnB) into Hs that results in overexpression of nifA at the amtB locus with simultaneous deletion of the amtB gene. (I) Genomic region of amtB before modification. (II) Transform with non-replicating plasmid and select on gentamicin (Gent). (III) Upon selection on Gent, the entire plasmid recombines into the genome. A counter-selection on hsvTK forces a second recombination event that loops out the rest of the plasmid that contains the hsvTK and Gent marker. (IV) amtB DS recombines with amtB DS to loop out the rest of the plasmid. (V) Genomic region of amtB after insertion. US=upstream sequence. DS=downstream sequence. CDS=coding sequence. Gent=gentamicin selection marker. hsvTK=human alpha herpesvirus 1 thymidine kinase counter-selection marker. Amp=ampicillin marker for selection in E. coli. P65=promoter P65. NifA=NifA coding sequence.

FIG. 5. Overview of conjugation process of Hs with E. coli BW29427 containing integration constructs.

FIGS. 6A, 6B. Pictures of dilution plates from representative conjugation experiments. (FIG. 6A) Selection for the first genomic crossover on gentamycin after conjugation, and (FIG. 6B) Counter selection in a nucleoside analog, dP6-(b-D-2-deoxyribofuranosyl)-3,4-dihydro8H-pyrimido[4,5-c][1,2]oxazin-7-one (dP).

FIG. 7. Schematic showing how an indicator strain behaves in the absence or presence of ammonium and how the indicator strain can be used to measure ammonium secretion from genetically modified Hs strains in an ammonia biosensor assay.

FIGS. 8A-8B. In vitro assays performed on engineered strain Hs12, in comparison to wild-type (wt) and ΔnifA negative control. (FIG. 8A) ammonia biosensor (indicator) assay—measures GFP fluorescence of Hs indicator strain as an estimate of extracellular ammonia concentration. (FIG. 8B) ¹⁵N dilution assay—measures percent of fixed nitrogen, ¹⁴N, incorporated into amino acids. P-value annotations are as follows: not significant (ns), p-value<0.1 (*), p-value<0.01 (**), and p-value<0.001 (***) based on a t-test.

FIG. 9. Depiction of an in planta assay to screen and characterize engineered microbes in the greenhouse.

FIG. 10. Nitrogen (N) dose response of corn shoot fresh weight. Arrow shows actual testing regime at 25 ppm N application rate. The application rate is the concentration of N in the fertilizer. The numbers under the curve arc p values.

FIG. 11. Dose response experiments. A standard curve of N fertilizer was added to corn plants and the resulting dry weight was measured. The regression slope is consistent from 2 different dose response experiments. The dose response for one experiment was 0.047 g/ppm N, and the second experiment showed 0.046 g/ppm N dry weight. The experiments showed a consistent response.

FIG. 12. Significant N supplement effect of engineered Hs12 (ΔglnK, PnifA::PCP25, amtB::P65:nifA) on corn biomass 28 days after planting (in-furrow inoculation) under 25 ppm N per application condition, p=0.021. The graph indicates that corn plants contacted with the non-genetically engineered wild type Hs0 (control) (and thus receiving no supplemental nitrogen from the bacteria) has a biomass of less than 6.6 g dry weight, while corn plants contacted with the genetically engineered Hs12 (and thus supplemented with 6.25 ppm nitrogen from the Hs12 microbe) has a biomass of >6.8 g dry weight.

FIG. 13. Significant N supplemented effect of engineered Hs12 (ΔglnK, PnifA::PCP25, amtB::P65:nifA) on corn biomass 28 days after planting (in-furrow inoculation) under a range of N application conditions, p=0.097. Mixed model and mean effect aggregated across N levels. The graph is similar to that of FIG. 12 except that the N supplement effect of Hs12 is aggregated across 0, 25, and 100 ppm N application.

### DETAILED DESCRIPTION

The present disclosure describes Herbaspirillum bacterial strains that are engineered to increase nitrogen fixation. The Herbaspirillum are genetically modified to express or overexpress one or more proteins and/or to attenuate expression of or knock out one or more genes involved in nitrogen fixation and its regulation, resulting in increased secretion of ammonia. When introduced to agricultural plants, the engineered bacteria form a close association with the host plants, which results in increased nitrogen availability to the plant and improved agronomic traits of the plants such as increased biomass production. In particular, nitrogen fertilizer application on agricultural plants can be reduced by introducing the engineered microbes of the disclosure to the agricultural plants.

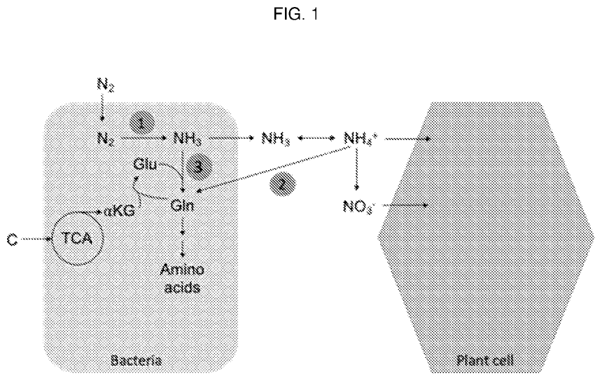

Herbaspirillum seropedicae (Hs) is a gram-negative diazotrophic (nitrogen fixing) endophyte, originally isolated from sorghum. The nitrogen fixation pathway in this organism is shown in FIG. 1. In order to increase the availability of fixed nitrogen to the plants, the following engineering strategies can be performed: (1) increase conversion of nitrogen to ammonia by increasing nitrogenase activity, (2) prevent re-assimilation of secreted ammonia into the cell by deleting the ammonia transporter, AmtB, and (3) reducing the incorporation of fixed ammonia into amino acids/biomass so that more of the fixed ammonia is secreted by the cells. In addition, the nitrogen fixation pathway is known to be tightly regulated by the presence of exogenous nitrogen, by ammonium or nitrate, through interactions with transcription factors (NtrC/NtrB) and regulatory proteins (GlnK) (Chubatsu et al. Plant and Soil 2012; 356(1-2):197-207).

The genome of Hs strain SmR1 has been annotated (GenBank NC_014323; Pedrosa et al. (2011) PLoS Genetics 7:e1002064). In Hs, structural and regulatory nitrogen fixation (nif) genes involved in biosynthesis, maturation, and assembly of active nitrogenase complex are clustered in a 40-kb continuous region that includes 46 open reading frames (orfs) and at least seven operons (Pedrosa et al. (2011) PLoS Genetics 7:e1002064). Independent analysis indicates that there may be as many as 55 open reading frames in Hs strain SmR1. The nif cluster includes genes necessary to fix nitrogen, including nitrogenase structural genes, genes encoding regulatory proteins that regulate nitrogenase production and activity, and genes encoding products for molybdenum uptake, electron transport, and metal cluster synthesis. Expression of the nif genes depends on polymerase sigma factor σ⁵⁴ and transcriptional activator NifA. The complete genome sequence of Hs Z78 used to create the genetically engineered Hs microbes of the disclosure is available (GenBank: CP011930.1).

Transcriptional activator NifA has three domains: an N-terminal region that appears to mediate inhibition of NifA in response to the level of fixed nitrogen by interacting with PII signal transduction proteins; a central region that interacts with the RNA polymerase holoenzyme containing sigma factor σ⁵⁴; and a C-terminal region with a conserved helix-turn-helix motif for DNA binding. NifA responds to the levels of fixed nitrogen and oxygen. Under increased levels of oxygen, NifA is inactivated. NifA is also regulated by a global nitrogen metabolism Ntr system in Hs. Proteins of the Ntr system regulate expression or repression of genes to mobilize nitrogen sources under ammonium ion limitation. The Ntr system includes: a uridylyltransferase encoded by glnD; signal transduction proteins of the PII family encoded by glnB and glnK; an ammonium transporter encoded by amtB; a glutamine synthetase (GS) encoded by glnA; an adenylyltransferase encoded by glnE; and the two-component regulatory system encoded by ntrB and ntrC. GlnB and GlnK are reversibly uridylylated by GlnD and regulate phosphorylation of NtrC by NtrB. Without being bound by any one hypothesis, the following can occur under nitrogen limiting conditions: active phosphorylated NtrC activates transcription of NifA and GlnK. GlnK is uridylylated by GlnD. Uridylylated GlnK interacts with the inhibitory N-terminal GAF domain of NifA, allowing NifA to be active to positively regulate expression of other nif genes.

The nitrogenase enzyme complex is responsible for fixing atmospheric nitrogen (N₂) by reducing it to ammonia (NH₃). The nitrogenase enzyme complex includes two metalloproteins, the iron-protein (Fe) and the molybdenum-iron (MoFe) protein. The iron molybdenum cofactor (FeMoco) is the site of N₂ reduction in the dinitrogenase protein. Nitrogen fixation is an energy-intensive endeavor, requiring a supply of magnesium adenosine triphosphate (MgATP). Reversible inhibition of nitrogenase activity can occur post-translationally once levels of extracellular ammonium (NH4⁺) increases. In one model, GlnK is deuridylylated in presence of high ammonium and binds to the AmtB ammonium transporter at the cell membrane. This triggers inhibition of nitrogenase by an unknown mechanism. The AmtB transporter transports ammonia into the cell. Mutants of AmtB in Hs have also been reported to exhibit nitrogenase activity even in the presence of ammonium; thus there is a potential role of the AmtB transporter in exogenous ammonia sensing (Noindorf et al., Archives of Microbiology 2006; 185(1):55-62).

The following aspects and options related to the current disclosure are now described in additional detail as follows: (i) Genetic Modifications; (ii) Assays; (iii) Compositions and Formulations; (iv) Methods of Use; (v) Exemplary Embodiments; (vi) Examples; (vii) Variants; and (viii) Closing Paragraphs.

### (i) Genetic Modifications

An Hs microbe of the present disclosure can be genetically modified to increase the amount of nitrogen available to a plant associated with the genetically modified microbe. In particular embodiments, modifications to the Hs microbe include: increasing nitrogenase activity; reducing or eliminating ammonium transport; reducing or eliminating nitrogen regulatory proteins; and reducing or attenuating glutamine synthase activity.

In particular embodiments, increasing nitrogenase activity includes expressing or overexpressing one or more heterologous NifA genes operably linked to a promoter. Particular embodiments provide for a heterologous expression cassette including an NifA gene inserted at a neutral site in the Hs genome. In particular embodiments, a neutral site includes any location in the Hs genome that allows insertion of heterologous nucleic acid without affecting transcription or translation of genes elsewhere in Hs and/or without affecting Hs growth or normal function. Particular embodiments provide for a heterologous expression cassette including an NifA gene inserted at the endogenous amtB gene. A heterologous nifA gene may also been inserted into other neutral sites including HSERO_RS20140, HSERO_RS06930, HSERO_RS04675, HSERO_RS17925, HSERO_RS20445, HSERO_RS10460, HSERO_RS17440 and HSERO_RS24900, operably linked to a promoter for expression or overexpression. In particular embodiments, the heterologous NifA gene is operably linked to a heterologous constitutive promoter. In particular embodiments, the heterologous NifA gene is operably linked to a P65 constitutive promoter. In particular embodiments, the heterologous NifA gene is operably linked to a CP32 constitutive promoter. In particular embodiments, the heterologous NifA gene is operably linked to a CP25 constitutive promoter. In yet other embodiments, the heterologous NifA gene is NifA from Herbaspirillum seropedicae, referred to herein as Hs NifA, or more particularly Herbaspirillum seropedicae Z78.

Regulation of NifA includes binding of NtrC to the NifA promoter (Chubatsu et al. Plant and Soil 2012; 356(1-2):197-207). Particular embodiments provide for insertion of a heterologous constitutive promoter to replace the endogenous Hs NifA promoter such that the heterologous constitutive promoter is operably linked to the endogenous Hs NifA gene. In particular embodiments, the heterologous constitutive promoter is CP25. In particular embodiments, the heterologous constitutive promoter is P65. In particular embodiments, the heterologous constitutive promoter is CP32. In particular embodiments, a genetic construct to obtain such genetic modification includes a polynucleotide sequence including (5′ to 3′) Hs NifA promoter upstream (US) homology arm—CP25 promoter—Hs NifA promoter downstream (DS) homology arm of SEQ ID NO: 5 or SEQ ID NO: 15. In particular embodiments, the heterologous constitutive promoter is CP32. In particular embodiments, a genetic construct to obtain such genetic modification includes a polynucleotide sequence including (5′ to 3′) Hs NifA promoter upstream (US) homology arm—CP25 promoter—Hs NifA promoter downstream (DS) homology arm having a sequence having at least 90% sequence identity to SEQ ID NO: 5 or to SEQ ID NO: 15.

Exogenous nitrogen represses the nitrogenase pathway. The present disclosure describes a microbial product that can be co-applied with fertilizer during planting, so exogenous nitrogen such as N-fertilizer could repress the nitrogen fixation pathway genes. Therefore, in some embodiments, the nitrogen repression of the nitrogenase pathway may be removed by downregulating expression of or deleting endogenous GlnK gene to increase nitrogen fixation in Hs. NifA is highly regulated by the signal transduction PII nitrogen regulatory proteins GlnB/GlnK (Chubatsu et al., Plant and Soil 2012; 356(1-2):197-207). Deleting GlnK could contribute to the removal of the NtrC mediated regulation of nifA, allowing the expression of NifA even in the presence of nitrogen. In particular embodiments, increasing nitrogenase activity includes downregulation of expression of the endogenous GlnK gene encoding P11-like nitrogen regulatory protein. Downregulation of the endogenous GlnK gene includes deleting (i.e., knocking out) the endogenous GlnK gene in an Hs microbe. In particular embodiments, a genetic construct to obtain such genetic modification includes a polynucleotide sequence including (5′ to 3′) glnK US homology arm—glnK DS homology arm of SEQ ID NO: 1 or SEQ ID NO: 11. In particular embodiments, a genetic construct to obtain such genetic modification includes a polynucleotide sequence including (5′ to 3′) glnK US homology arm—glnK DS homology arm having at least 90% sequence identity to SEQ ID NO: 1 or to SEQ ID NO: 11.

In particular embodiments, an Hs microbe is genetically modified to downregulate the expression of or delete (e.g., knock out) the endogenous amtB gene encoding ammonium transporter AmtB to increase the amount of nitrogen available to a plant associated with the genetically modified Hs microbe. In particular embodiments, downregulating the expression of or deleting the endogenous amtB gene encoding ammonium transporter AmtB reduces or prevents re-assimilation of secreted ammonium, thus increasing the amount of extracellular ammonium for uptake by a plant. In particular embodiments, a genetic construct to obtain a deletion of endogenous amtB gene includes a polynucleotide sequence including (5′ to 3′) amtB US homology arm—amtB DS homology arm of SEQ ID NO: 4 or SEQ ID NO: 14. In particular embodiments, a genetic construct to obtain a deletion of endogenous amtB gene includes a polynucleotide sequence including (5′ to 3′) amtB US homology arm—amtB DS homology arm having at least 90% sequence identity to SEQ ID NO: 4 or to SEQ ID NO: 14. Particular embodiments provide for replacement of the endogenous amtB gene with an exogenous NifA expression cassette such that expression of AmtB is reduced or eliminated and a heterologous NifA is expressed. In particular embodiments, a genetic construct to obtain a replacement of endogenous amtB gene with an exogenous NifA expression cassette includes a polynucleotide sequence including (5′ to 3′) amtB US homology arm—P65 promoter—Hs NifA CDS—amtB DS homology arm of SEQ ID NO: 2 or SEQ ID NO: 12. In particular embodiments, a genetic construct to obtain a replacement of endogenous amtB gene with an exogenous Hs NifA expression cassette includes a polynucleotide sequence including (5′ to 3′) amtB US homology arm—P65 promoter—Hs NifA CDS—amtB DS homology arm having at least 90% sequence identity to SEQ ID NO: 2 or to SEQ ID NO: 12. In particular embodiments, a genetic construct to obtain a replacement of endogenous amtB gene with an exogenous NifA expression cassette includes a polynucleotide sequence including (5′ to 3′) amtB US homology arm—CP32 promoter—Hs NifA CDS—amtB DS homology arm of SEQ ID NO: 3 or SEQ ID NO: 13. In particular embodiments, a genetic construct to obtain a replacement of endogenous amtB gene with an exogenous NifA expression cassette includes a polynucleotide sequence including (5′ to 3′) amtB US homology arm—CP32 promoter—Hs NifA CDS—amtB DS homology arm having at least 90% sequence identity to SEQ ID NO: 3 or to SEQ ID NO: 13.

In particular embodiments, the genetically modified Hs microbe includes: (a) a deletion of endogenous glnK gene encoding PII-like nitrogen regulatory protein; (b) a heterologous nifA gene encoding transcriptional activator NifA operably linked to a first heterologous constitutive promoter; (c) deletion of endogenous amtB gene encoding ammonium transporter AmtB; and (d) endogenous nifA gene operably linked to a second heterologous constitutive promoter, wherein deletion of the endogenous amtB gene includes replacement of the endogenous amtB gene with the heterologous nifA gene operably linked to the first heterologous constitutive promoter such that expression of AmtB is reduced or eliminated, and wherein the second heterologous constitutive promoter replaces the endogenous nifA promoter. In particular embodiments, the first heterologous constitutive promoter is P65, the second heterologous constitutive promoter is CP25, and the genetically modified Hs microbe is Hs12. In particular embodiments, the first heterologous constitutive promoter is P65, the second heterologous constitutive promoter is CP32, and the genetically modified Hs microbe is Hs8. In yet other embodiments, the heterologous NifA gene is Hs NifA.

In particular embodiments, the genetically modified Hs microbe includes: (a) a deletion of endogenous glnK gene encoding PII-like nitrogen regulatory protein; (b) a heterologous nifA gene encoding transcriptional activator NifA operably linked to a heterologous constitutive promoter; and (c) deletion of endogenous amtB gene encoding ammonium transporter AmtB, wherein deletion of the endogenous amtB gene includes replacement of the endogenous amtB gene with the heterologous nifA gene operably linked to the heterologous constitutive promoter such that expression of AmtB is reduced or eliminated. In particular embodiments, the heterologous constitutive promoter includes CP32 and the genetically modified Hs microbe is Hs3. In yet other embodiments, the heterologous NifA gene is Hs NifA.

In particular embodiments, the genetically modified Hs microbe includes: (a) a deletion of endogenous ntrC gene encoding nitrogen regulatory protein NtrC; (b) a heterologous nifA gene encoding transcriptional activator NifA operably linked to a first heterologous constitutive promoter; (c) deletion of endogenous amtB gene encoding ammonium transporter AmtB; and (d) endogenous nifA gene operably linked to a second heterologous constitutive promoter, wherein deletion of the endogenous amtB gene includes replacement of the endogenous amtB gene with the heterologous nifA gene operably linked to the first constitutive promoter such that expression of AmtB is reduced or eliminated, and wherein the second heterologous constitutive promoter replaces the endogenous nifA promoter. In particular embodiments, the first heterologous constitutive promoter is CP32, the second heterologous constitutive promoter is CP25, and the genetically modified Hs microbe is Hs4. In particular embodiments, the first heterologous constitutive promoter is P65, the second heterologous constitutive promoter is CP25, and the genetically modified Hs microbe is Hs11. In yet other embodiments, the heterologous NifA gene is Hs NifA.

In particular embodiments, the genetically modified Hs microbe includes: (a) a heterologous nifA gene encoding transcriptional activator NifA operably linked to a heterologous constitutive promoter; and (b) deletion of endogenous amtB gene encoding ammonium transporter AmtB, wherein deletion of the endogenous amtB gene includes replacement of the endogenous amtB gene with the heterologous nifA gene operably linked to the heterologous constitutive promoter such that expression of AmtB is reduced or eliminated. In particular embodiments, the heterologous constitutive promoter includes CP32 and the genetically modified Hs microbe is Hs2. In particular embodiments, the heterologous constitutive promoter includes P65 and the genetically modified Hs microbe is Hs5. In yet other embodiments, the heterologous NifA gene is Hs NifA.

In particular embodiments, the genetically modified Hs microbe includes: (a) a deletion of endogenous glnK gene encoding PII-like nitrogen regulatory protein; (b) a heterologous nifA gene encoding transcriptional activator NifA operably linked to a heterologous constitutive promoter; and (c) deletion of endogenous amtB gene encoding ammonium transporter AmtB, wherein deletion of the endogenous amtB gene includes replacement of the endogenous amtB gene with the heterologous nifA gene operably linked to the heterologous constitutive promoter such that expression of AmtB is reduced or eliminated. In particular embodiments, the heterologous constitutive promoter is P65 and the genetically modified Hs microbe is Hs6.

In particular embodiments, the genetically modified Hs microbe includes: (a) a deletion of endogenous ntrC gene encoding nitrogen regulatory protein NtrC; (b) a heterologous nifA gene encoding transcriptional activator NifA operably linked to a heterologous constitutive promoter; and (c) deletion of endogenous amtB gene encoding ammonium transporter AmtB, wherein deletion of the endogenous amtB gene includes replacement of the endogenous amtB gene with the heterologous nifA gene operably linked to the heterologous constitutive promoter such that expression of AmtB is reduced or eliminated. In particular embodiments, the heterologous constitutive promoter is CP32, and the genetically modified Hs microbe is Hs7. In particular embodiments, the heterologous constitutive promoter is P65, and the genetically modified Hs microbe is Hs9 or Hs10. In yet other embodiments, the heterologous NifA gene is Hs NifA.

In particular embodiments, the genetically modified Hs microbe includes: (a) deletion of endogenous amtB gene encoding ammonium transporter AmtB; and (b) endogenous nifA gene operably linked to a heterologous constitutive promoter, wherein the heterologous constitutive promoter replaces the endogenous nifA promoter. In particular embodiments, the heterologous constitutive promoter is CP25.

In particular embodiments, the genetically modified Hs microbe includes endogenous nifA gene operably linked to a heterologous constitutive promoter, wherein the heterologous constitutive promoter replaces the endogenous nifA promoter. In particular embodiments, the heterologous constitutive promoter is CP25.

In particular embodiments, the genetically modified Hs microbe includes: (a) a deletion of endogenous ntrC gene encoding nitrogen regulatory protein NtrC; and (b) deletion of endogenous amtB gene encoding ammonium transporter AmtB.

NtrC is essential for activating the glnA promoter under nitrogen limiting conditions, which drives expression of the glutamine synthetase glnA gene to assimilate ammonia into amino acids. NtrC also appears to play a role in not only GS expression but also GS activity, in particular, as it relates to nitrate assimilation. Therefore, downregulating expression of or deleting the endogenous ntrC gene may reduce or prevent the assimilation of ammonia into amino acids and/or biomass of an Hs microbe, allowing more ammonia to be secreted from the Hs microbe for uptake by a plant. In particular embodiments, an Hs microbe is genetically modified to downregulate expression of or delete (e.g., knock out) the endogenous NtrC gene encoding nitrogen regulatory protein NtrC to increase the amount of nitrogen available to a plant associated with the genetically modified Hs microbe. In particular embodiments, a genetic construct to obtain such genetic modification includes a polynucleotide sequence including (5′ to 3′) ntrC US homology arm—ntrC DS homology arm of SEQ ID NO: 6 or SEQ ID NO: 16. In particular embodiments, a genetic construct to obtain such genetic modification includes a polynucleotide sequence including (5′ to 3′) ntrC US homology arm—ntrC DS homology arm having at least 90% sequence identity to SEQ ID NO: 6 or to SEQ ID NO: 16.

In particular embodiments, the genetically modified Hs microbe includes a deletion of endogenous nifA gene encoding transcriptional activator NifA and serves as a negative control. In particular embodiments, a genetic construct to obtain such genetic modification includes a polynucleotide sequence including (5′ to 3′) nifA US homology arm—nifA DS homology arm of SEQ ID NO: 9 or SEQ ID NO: 19. In particular embodiments, a genetic construct to obtain such genetic modification includes a polynucleotide sequence including (5′ to 3′) nifA US homology arm—nifA DS homology arm having at least 90% sequence identity to SEQ ID NO: 9 or to SEQ ID NO: 19.

In particular embodiments, the genetically modified Hs microbe includes a deletion of endogenous nifH gene encoding a dinitrogenase reductase and serves as a control. In particular embodiments, a genetic construct to obtain such genetic modification includes a polynucleotide sequence including (5′ to 3′) nifH US homology arm—nifH DS homology arm of SEQ ID NO: 8 or SEQ ID NO: 18. In particular embodiments, a genetic construct to obtain such genetic modification includes a polynucleotide sequence including (5′ to 3′) nifH US homology arm—nifH DS homology arm having at least 90% sequence identity to SEQ ID NO: 8 or SEQ ID NO: 18.

Compositions including microbes that are genetically engineered to decrease the expression of endogenous NifH are provided. In some embodiments the expression is decreased by inserting a heterologous promoter such that it is operably linked to the endogenous nifH gene to decrease activity and/or expression of NifH compared to the native nifH promoter. In some embodiments, the nifH gene can be partially or fully deleted from the microbe's genome.

In particular embodiments, an Hs microbe is genetically modified to reduce glutamine synthetase (GS, encoded by glnA) expression and/or activity to increase the amount of nitrogen available to a plant associated with the genetically modified Hs microbe. In particular embodiments, reducing GS activity reduces or prevents the amount of ammonia that is assimilated into amino acids and/or biomass of the microbe, thus increasing the amount of ammonia that is secreted from Hs for uptake by a plant.

In particular embodiments, reducing GS expression and/or activity includes reducing GS expression by regulating the strength of the promoter operably linked to glnA. Particular embodiments provide for regulating promoter strength by replacing the endogenous Hs glnA promoter with an exogenous promoter that drives lower expression of GS as compared to the native endogenous glnA promoter. Particular embodiments provide for regulating promoter strength by mutating the endogenous Hs glnA promoter so that it drives lower expression of GS as compared to the native endogenous glnA promoter.

In particular embodiments, reducing GS expression and/or activity includes reducing GS expression by regulating ribosomal binding site strength. Particular embodiments provide for endogenous Hs glnA gene operably linked to a ribosomal binding site (RBS) modified to decrease translation efficiency of the GS as compared to the endogenous Hs glnA gene operably linked to the corresponding native unmodified RBS. Particular RBS sequences are disclosed in: Levin-Karp et al. (2013) ACS synthetic biology 2(6):327-336; Wang et al. (2011) Nature communications 2(1):1-9; and Salis et al. (2009) Nature biotechnology 27(10):946-950).

In particular embodiments, reducing GS expression and/or activity includes decreasing GS protein half-life via a degradation tag. Degradation tags include short peptide sequences that mark a protein for degradation by the cell's protein recycling machinery. In particular embodiments, a degradation tag effectively decreases the protein half-life or the typical length of time that a protein will exist in a cell once it is translated. Protein half-life includes the interval of time it takes for the level of a protein to decay to half its initial value. In particular embodiments, a degradation tag can decrease the concentration of the protein in the cell. For example, a degradation tag includes an ssRA tag. During translation a ribosome can get stuck on a truncated mRNA without a normal termination codon, and the ribosome cannot detach from the defective mRNA. In particular embodiments, a special type of RNA known as ssRA (small stable RNA A) or tmRNA (transfer-messenger RNA) rescues the ribosome by adding an eleven codon degradation tag followed by a stop codon. This allows the ribosome to break free and continue functioning. The tagged, incomplete protein then gets degraded by proteases ClpXP and ClpAP. In particular embodiments, a degradation tag includes an ssRA/tmRNA tag including the sequence AANDENYALAA (SEQ ID NO: 10) (Keiler et al. (2000) Proceedings of the National Academy of Sciences 97(14):7778-7783). In particular embodiments, other degradation tags can vary in the final three amino acids (AAV, ASV, LVA, LAA) and result in different protein half-lives (Andersen et al. (1998) Applied and environmental microbiology 64(6):2240-2246). Other degradation tags have also been described (Cameron and Collins (2014) Nat Biotechnol 32(12):1276-1281).

In particular embodiments, reducing GS expression and/or activity includes replacing the endogenous Hs glnA promoter with an inducible promoter. For example, the inducible promoter includes Ptac promoter, regulated by the lac repressor, and inducible by a compound such as isopropyl β-D-thiogalactopyranoside (IPTG) (de Boer et al. Proc. Natl. Acad. Sci USA 1983; 80:21-25). In particular embodiments, a genetic construct to obtain a replacement of the the endogenous Hs glnA promoter with an inducible promoter includes a polynucleotide sequence including (5′ to 3′) glnA promoter US homology arm—lac promoter—lacI CDS—tac promoter—glnA promoter DS homology arm of SEQ ID NO: 7 or SEQ ID NO: 17. In particular embodiments, a genetic construct to obtain a replacement of the the endogenous Hs glnA promoter with an inducible promoter includes a polynucleotide sequence including (5′ to 3′) glnA promoter US homology arm—lac promoter—lacI CDS—tac promoter—glnA promoter DS homology arm having at least 90% sequence identity to SEQ ID NO: 7 or to SEQ ID NO: 17. In particular embodiments, the genetically modified Hs microbe includes: (a) a deletion of endogenous glnK gene encoding PII-like nitrogen regulatory protein; (b) a heterologous nifA gene encoding transcriptional activator NifA operably linked to a heterologous constitutive promoter; (c) deletion of endogenous amtB gene encoding ammonium transporter AmtB; and (d) endogenous glnA gene operably linked to an inducible promoter, wherein deletion of the endogenous amtB gene includes replacement of the endogenous amtB gene with the heterologous nifA gene operably linked to the heterologous constitutive promoter such that expression of AmtB is reduced or eliminated, and wherein the inducible promoter replaces the endogenous glnA promoter. In particular embodiments, the heterologous constitutive promoter includes CP32. In particular embodiments, the inducible promoter is Ptac and the Hs microbe further includes an expression cassette including a lacI gene encoding a lac repressor. In particular embodiments, the lacI gene is operably linked to a lac promoter. In particular embodiments, the lacI gene is transcribed in the opposite direction from that of the glnA gene operably linked to Ptac. In yet other particular embodiments, the heterologous nifA gene is Hs NifA.

In particular embodiments, reducing GS activity includes removing the adenylyl-removing domain of GlnE. The adenylyl transferase GlnE transfers and removes the adenylation of GS. When GS is adenylated, it is inactive. Therefore, GS may be kept inactive to increase the amount of ammonia secreted from an Hs microbe as described herein by truncating an amino-terminal region of glnE such that glnE is unable to de-adenylate GS, keeping GS inactive. Particular embodiments provide for an amino (N)-terminal truncation of the endogenous glnE gene. In particular embodiments, the N-terminal truncation is from amino acid residue 1 to 260 of endogenous Hs glnE gene.

As used herein, the term “genetically modified” or “genetically engineered” refers to the addition of extra genetic material in the form of DNA or RNA into the total genetic material in a cell or modification of the genome of a cell such that the genome contains insertions, deletions, mutations, and/or rearrangements of the genomic DNA after introduction of extra genetic material as compared to a cell that is not genetically modified. The terms “genetically modified microbe”, “genetically engineered microbe”, “engineered microbe”, and “modified microbe” are used interchangeably. The term “genetically modified” or “genetically engineered” also refers to multiple genetic modifications, e.g., 2, 3, 4, 5, 6, 7, 8, 9, 10 or more genetic modifications, for example, a microbe which has an exogenous gene introduced for expression of a protein, and a modification, such as a gene knockout, which reduces expression of an endogenous microbe gene.

The term “heterologous” refers to a molecule (e.g., nucleic acid, gene, RNA, protein) that originates outside a microbe and is introduced into a microbe by genetic engineering. In particular embodiments, a heterologous molecule can include sequences that are native to a microbe to which the heterologous molecule is introduced; however, the heterologous molecule is synthesized outside the microbe and introduced into the microbe. For example, the disclosure includes a genetically modified Hs microbe including at least one heterologous nifA gene. In a particular embodiment, the nifA sequence is native to Hs but is introduced into an Hs microbe by genetic engineering. In particular embodiments, a heterologous molecule can include sequences, including a nifA gene, which are not native to a microbe to which the heterologous molecule is introduced; the heterologous molecule is synthesized outside the microbe and introduced into the microbe. For example, the disclosure includes a genetically modified Hs microbe including a heterologous nifA expression cassette. The heterologous nifA expression cassette can include a promoter-nifA gene combination not found naturally (not native) to the Hs microbe. As another example, the disclosure includes a genetically modified Hs microbe including endogenous nifA gene operably linked to a heterologous constitutive promoter. The constitutive promoter is not native to Hs and is synthesized outside the Hs microbe. Upon introduction into the Hs microbe, the heterologous constitutive promoter is operably linked to the endogenous nifA gene by homologous recombination to replace the endogenous nifA promoter. The term “exogenous” can be used interchangeably with “heterologous”.

The term “endogenous” refers to a molecule (e.g., nucleic acid, gene, RNA, protein) that is naturally occurring or naturally produced in a given microbe. For example, the disclosure includes a genetically modified Hs microbe with deletion of the endogenous ammonium transporter amtB gene. The endogenous amtB gene is the amtB gene that is naturally found in Hs. The term “native” can be used interchangeably with “endogenous”.

In particular embodiments, the term “gene” refers to a nucleic acid sequence (used interchangeably with polynucleotide or nucleotide sequence) that encodes, e.g., a protein associated with nitrogen fixation or regulation of nitrogen fixation, as described herein. This definition includes various sequence polymorphisms, mutations, and/or sequence variants wherein such alterations do not substantially affect the function of the encoded protein. The nucleic acid sequences can include both the full-length nucleic acid sequences as well as non-full-length sequences derived from a full-length protein. The sequences can also include degenerate codons of the native sequence or sequences that may be introduced to provide codon preference in a specific microbe. In particular embodiments, the term “gene” may include not only coding sequences but also regulatory regions such as promoters, enhancers, 5′ UTR, 3′UTR, termination regions, and non-coding regions. Gene sequences encoding a molecule can be DNA or RNA that directs the expression of the molecule. These nucleic acid sequences may be a DNA strand sequence that is transcribed into RNA or an RNA sequence that is translated into protein. An essential gene is an endogenous gene (e.g., endogenous to a microbe) that produces a polypeptide (e.g., an essential protein) that is necessary for the growth and/or viability of a microbe.

“Encoding” refers to the property of specific sequences of nucleotides in a gene, such as a complementary DNA (cDNA), or a messenger RNA (mRNA), to serve as templates for synthesis of other macromolecules such as a defined sequence of amino acids or a functional polynucleotide (e.g., siRNA). In particular embodiments, a gene encodes or codes for a protein if the gene is transcribed into mRNA and translation of the mRNA produces the protein in a cell or other biological system. A “gene sequence encoding a protein” includes all nucleotide sequences that are degenerate versions of each other and that code for the same primary amino acid sequence or amino acid sequences of substantially similar form and function.

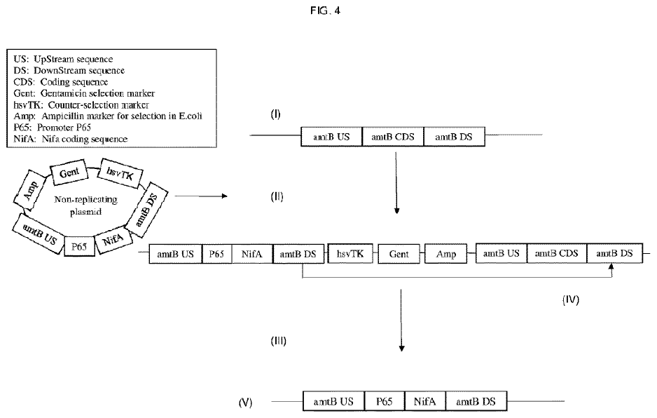

A “gene deletion” or “gene knockout” refers to a combination of genetic techniques that can render a specific gene inoperable or inactive. In particular embodiments, a gene deletion reduces or eliminates expression of a polypeptide encoded by the gene. In particular embodiments, the expression of the gene is substantially reduced or eliminated. Substantially reduced means that the expression of a gene is reduced by at least 80%, at least 90%, at least 95%, or at least 98% when compared to an endogenous level of expression of the gene. Expression of a gene can be determined by a suitable technique (e.g., by measuring transcript or expressed protein levels). In particular embodiments, a gene is deleted by introducing one or more mutations that disable the function of a protein encoded by the gene. In particular embodiments, a gene is partially or completely removed from the genome of a microbe. In particular embodiments, an endogenous gene is deleted by replacing the gene with a different gene (e.g., the endogenous Hs amtB gene is replaced with a heterologous nifA expression cassette) or a selectable marker (e.g., antibiotic selectable marker, auxotrophic selectable marker). Replacing an endogenous gene in a microbe may occur by homologous recombination, which includes introducing a genetic construct into the microbe, where the genetic construct includes homology arms having homology to target sequences of the gene to be deleted. In particular embodiments, the genetic construct includes a non-homologous polynucleotide flanked by two polynucleotide regions of homology (i.e., the upstream and downstream homology arms), such that homologous recombination between target sequences of the gene to be deleted and the two flanking homology arms results in insertion of the non-homologous polynucleotide at the target region (see FIG. 4). In particular embodiments, the target sequences homologous to the upstream and downstream homology arms include sequence 5′ of the coding sequence and 3′ of the coding sequence of the gene to be deleted, respectively. However, one of skill in the art will recognize that the upstream and downstream homology arms can have homology to other target sequences such that less than the full-length coding sequence of a gene is deleted, a combination of a portion of the full-length coding sequence and sequences upstream (5′) and/or downstream (3′) of the coding sequence is deleted, a combination of the full-length coding sequence and sequences upstream (5′) and/or downstream (3′) of the coding sequence is deleted, or any other variation on deletion of a gene, as long as expression of the gene is reduced or eliminated. In particular embodiments, the homology arms include sequence having at least 50% sequence identity to a target sequence with which homologous recombination is desired. In particular embodiments, a homology arm includes sequence having at least 60%, at least 70%, at least 80%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to a target sequence. In particular embodiments, each homology arm may include 100 nucleotides (nt), 500 nt, 750 nt, 1000 nt, 1250 nt, 1500 nt, 2000 nt, 2500 nt, 3000 nt, or more. In particular embodiments, the non-homologous polynucleotide flanked by the upstream and downstream homology arms includes a promoter, a gene, a terminator, a selectable marker, a counter-selectable marker, or a combination thereof. In particular embodiments, deletion of an endogenous gene in a microbe by homologous recombination includes deletion of the gene without any heterologous sequences inserted at the target sequences, such as heterologous expression cassettes including selectable or counter-selectable markers. In particular embodiments, a gene is deleted by disabling an endogenous promoter, operon or regulatory element that is essential for transcription or translation of the gene. Disabling an endogenous promoter, operon, or regulatory element can include introducing mutations into the promoter, operon, or regulatory element or deleting a portion or all of the promoter, operon, or regulatory element as described herein such that transcription or translation of the gene is reduced or eliminated.

Any suitable technique can be used to generate a gene deletion in a microbe. In particular embodiments, a gene deletion in Hs is mediated by in vivo homologous recombination. In particular embodiments, homologous recombination allows targeted insertion of a heterologous nucleic acid at a genomic site to disrupt a gene (e.g., coding region, promoter). In particular embodiments, homologous recombination allows disruption of a gene but does not introduce a heterologous nucleic acid into the gene being disrupted. In particular embodiments, homologous recombination can be facilitated by including homology regions in a genetic construct and introducing the genetic construct into an Hs microbe. Homology regions (i.e., homology arms) are homologous to sequences at a genomic site targeted for disruption. In particular embodiments, homology arms refer to segments of nucleic acid included in a genetic construct that are 100% identical to a region of a gene that is being modified. In particular embodiments, 100% identity may not be required to achieve homologous recombination (e.g., at least 90% identity may be sufficient).

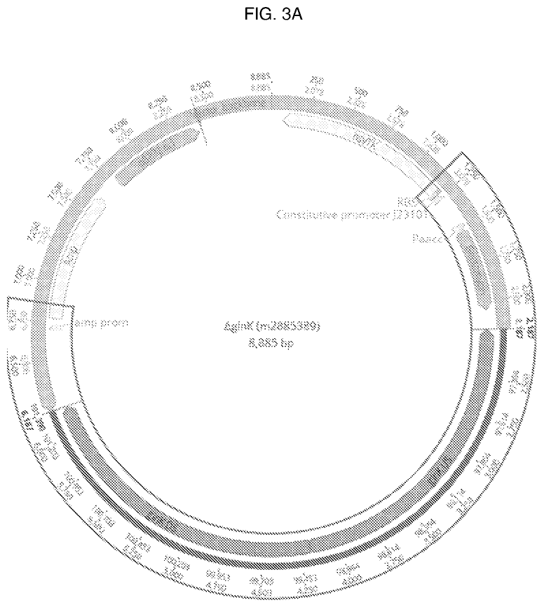

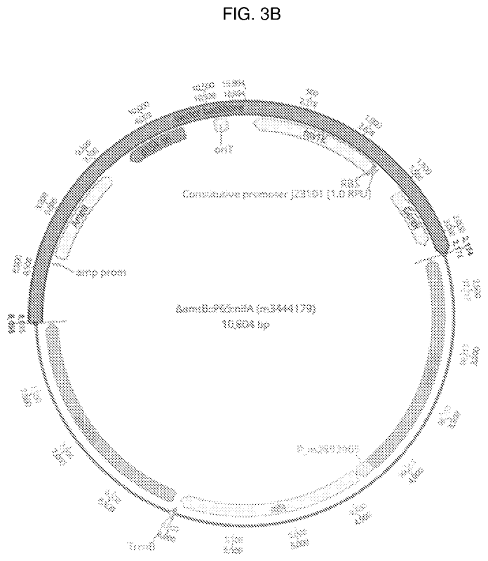

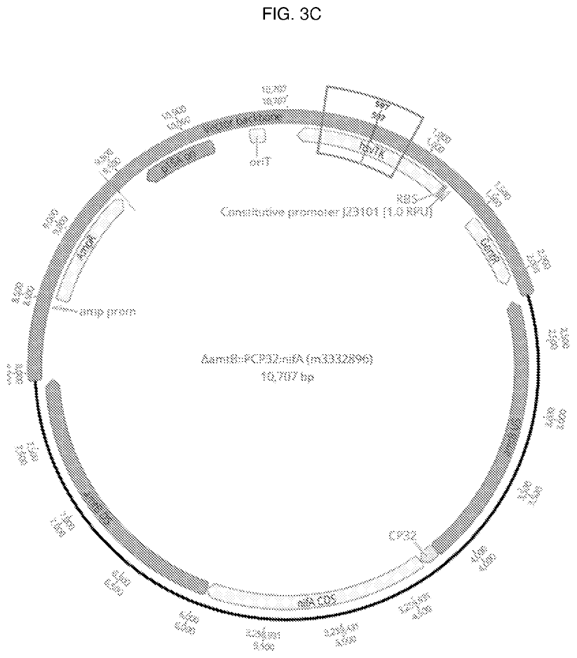

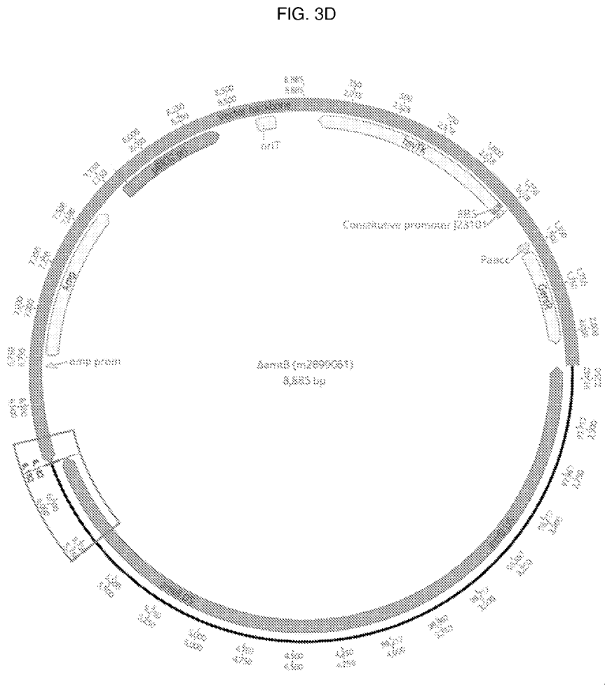

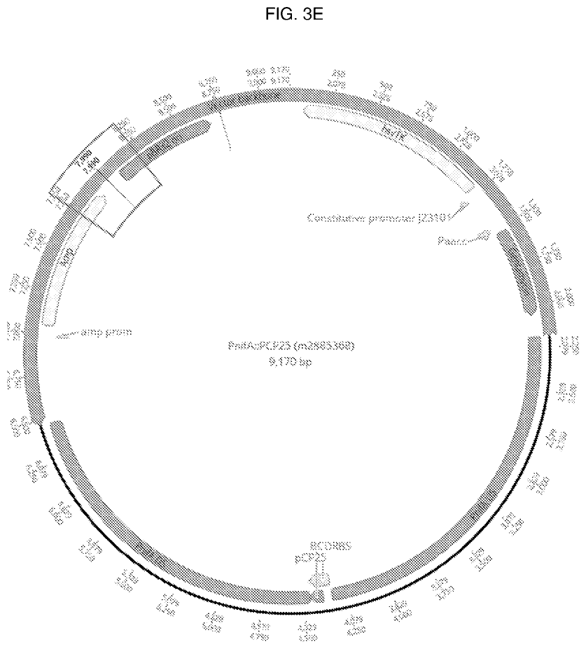

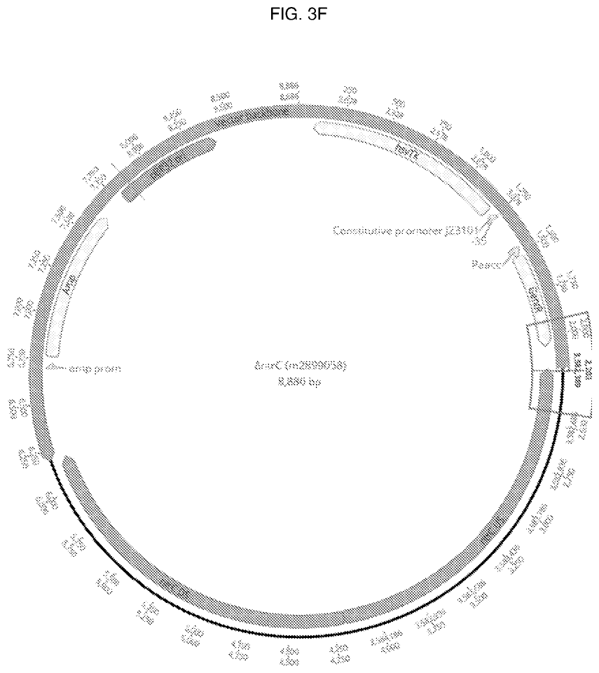

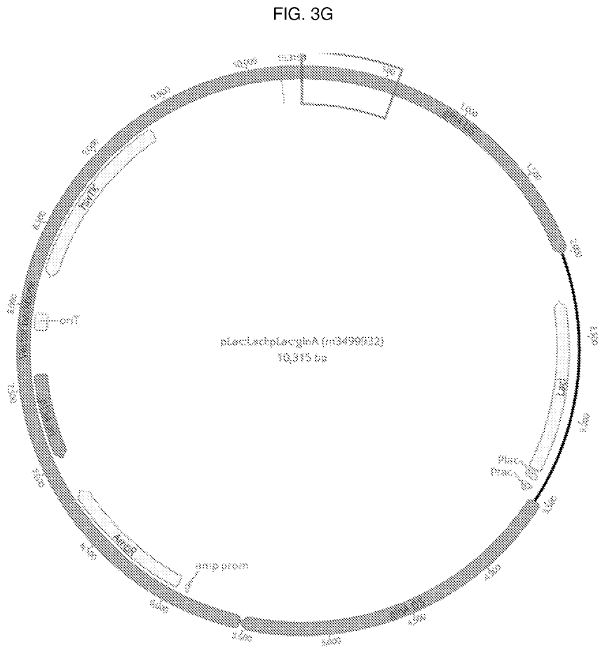

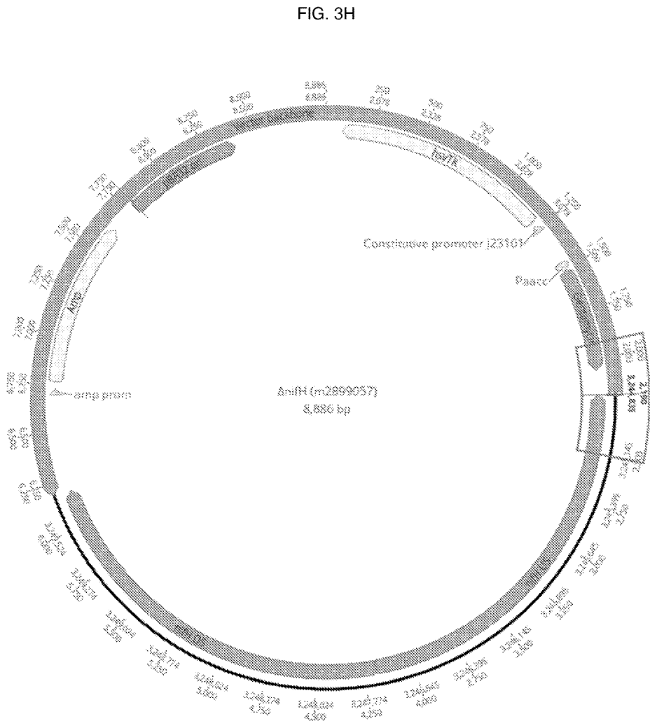

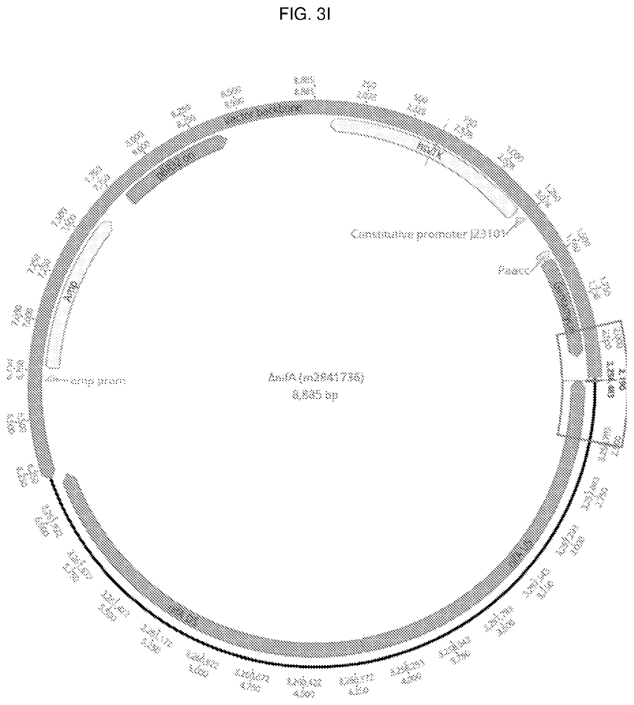

Homology regions cause the genetic construct to align next to the targeted genomic region, and portions of nucleic acid from the genetic construct are swapped into the region by homologous recombination (FIG. 4). In particular embodiments, a genetic construct may include an upstream (US) homology arm with homology to an upstream region of an endogenous gene targeted for disruption in a microbe, and a downstream (DS) homology arm with homology to a downstream region of an endogenous gene targeted for disruption in a microbe. In particular embodiments, a US homology arm may include nucleic acid sequence having 100% sequence identity to sequence 5′ of the start codon of an endogenous gene targeted for disruption in a microbe. In particular embodiments, a DS homology arm may include nucleic acid sequence having 100% sequence identity to sequence 3′ of the stop codon of an endogenous gene targeted for disruption in a microbe. In particular embodiments, a homology arm may include 20-3000 base pairs (bp), or 100-2500 bp, or 200-2000 bp. In particular embodiments, a homology arm may include 2000 bp. In particular embodiments, homology arms are included in SEQ ID NOs: 1-9 and 11-19. For example, the genetic construct of FIG. 3A includes a 2000 bp US glnK homology arm and a 2000 bp DS glnK homology arm. Upon homologous recombination in an Hs microbe, the genetic construct including a gentamicin resistance selectable marker and human herpes simplex virus thymidine kinase (hsvTK) counter-selectable marker is inserted at the glnK coding region. The insertion of the genetic construct can be selected by growing the microbe in media containing gentamicin. Cells of the genetically modified microbe that have undergone a second homologous recombination event to loop out the genetic construct backbone can subsequently be obtained by growing the microbe in media containing the nucleoside analog dP6-(b-D-2-deoxyribofuranosyl)-3,4-dihydro8H-pyrimido[4,5-c][1,2]oxazin-7-one (dP) to select against cells that still retain the genetic construct backbone. Similar genetic constructs can be used to: replace the endogenous amtB gene with a P65 promoter NifA coding sequence (CDS) expression cassette (FIG. 3B); replace the endogenous amtB gene with a CP32 promoter NifA CDS expression cassette (FIG. 3C); delete the amtB gene (FIG. 3D); replace the endogenous Hs nifA promoter with a CP25 promoter (FIG. 3E); delete the ntrC gene (FIG. 3F); replace the endogenous Hs glnA promoter with a Plac-lacI expression cassette and Ptac promoter for inducible expression of glnA (FIG. 3G); delete the nifH gene (FIG. 3H); and delete the nifA gene (FIG. 3I).

The terms “peptide,” “oligopeptide,” “polypeptide,” “polyprotein,” and “protein” are used interchangeably herein and refer to a polymeric form of amino acids of any length, which can include coded and non-coded amino acids, chemically or biochemically modified or derivatized amino acids, and polypeptides having modified peptide backbones.

The term “recombinant” refers to a particular DNA or RNA sequence that is the product of various combinations of cloning, restriction, and/or ligation steps resulting in a construct having a structural coding or non-coding sequence distinguishable from homologous sequences found in natural systems. Generally, DNA sequences encoding the structural coding sequence can be assembled from cDNA fragments and short oligonucleotide linkers, or from a series of oligonucleotides, to provide a synthetic gene which is capable of being expressed in a recombinant transcriptional unit. Such sequences can be provided in the form of an open reading frame uninterrupted by internal non-translated sequences, or introns. Genomic DNA including the relevant sequences could also be used. Sequences of non-translated DNA may be present 5′ or 3′ of the open reading frame, where such sequences do not interfere with manipulation or expression of the coding regions. In particular embodiments, the term “recombinant” polynucleotide or nucleic acid refers to one which is not naturally occurring or is made by the artificial combination of two otherwise separated segments of sequence. This artificial combination is often accomplished by either chemical synthesis means, or by the artificial manipulation of isolated segments of nucleic acids, e.g., by genetic engineering techniques. For example, such is usually done to replace a codon with a redundant codon encoding the same or a conservative amino acid, while typically introducing or removing a sequence recognition site. Alternatively, it is performed to join together nucleic acid segments of desired functions to generate a desired combination of functions.

A “genetic construct” includes a recombinant nucleic acid, generally recombinant DNA, which has been generated for the purpose of the expression of a specific polynucleotide sequence(s) or is to be used in the construction of other recombinant polynucleotide sequences. In particular embodiments, the term genetic construct includes plasmids and vectors. In particular embodiments, a genetic construct can be circular or linear. Genetic constructs can include, for example, an origin of replication, a multicloning site, a selectable marker, and/or a counter-selectable marker. In particular embodiments, a genetic construct includes an expression cassette. In particular embodiments, an expression cassette of the disclosure includes: (a) a heterologous promoter; and (b) an Hs gene selected from: nifA; glnA; nifB, fdxN, hesB, 2867, nifZ, nifZ1, 2864, nifS2, fixU, 2861, 2860, fdx, and/or 2858 of Hs nifB operon; nifS, nifU, 2831, hesB1, 2833, fdx, nifV, nifW, fixA, fixB, fixC, and/or fixX of Hs nifS operon; genes of Hs limB operon; or genes of Hs hypo_2870 operon.

In particular embodiments, cells expressing a selectable marker can grow in the presence of a selective agent or under a selective growth condition. Examples of selectable markers include antibiotic resistance markers (e.g., chloramphenicol resistance, erythromycin resistance, ampicillin resistance, carbenicillin resistance, kanamycin resistance, spectinomycin resistance, streptomycin resistance, tetracycline resistance, bleomycin resistance, and polymyxin B resistance), markers that complement an essential gene (e.g., diaminopimelic acid auxotrophy (dapD), thymidine auxotrophy (thyA), proline auxotrophy (proBA), glycine auxotrophy (glyA), carbon source auxotrophy (TpiA)), chemical resistance (e.g., tellurite resistance, Fabl for triclosan resistance, bialaphos herbicide resistance, mercury resistance, arsenic resistance), and visual markers (e.g., green fluorescent protein (GFP), luciferase, 0-galactosidase (lacZ)). In particular embodiments, a genetic construct of the disclosure includes a chloramphenicol acetyl transferase resistance gene (CAT) operably linked to a chloramphenicol responsive promoter (PCAT) and terminator (TCAT) from the Staphylococcus plasmid pC194 (Horinouchi and Weisblum. J Bacteriol. 1982; 150(2): 815-825). In particular embodiments, cells may be positively selected that have lost expression of a counter-selectable marker (i.e., cells expressing a counter-selectable marker are selected against). Examples of genes encoding counter-selectable markers include: sacB (gene encoding levansucrase that converts sucrose to levans, which is harmful to bacteria); rpsL (strA) (encodes the ribosomal subunit protein (S12) target of streptomycin); tetAR (confers sensitivity to lipophilic compounds such as fusaric and quinalic acids); pheS (encodes the a subunits of Phe-tRNA synthetase, which renders bacteria sensitive to p-chlorophenylalanine, a phenylalanine analog); thyA (encodes thymidilate synthetase, which confers sensitivity to trimethoprim and related compounds); lacY (encodes lactose permease, which renders bacteria sensitive to t-o-nitrophenyl-β-D-galactopyranoside); gata-1 (encodes a zinc finger DNA-binding protein which inhibits the initiation of bacterial replication); and ccdB (encodes a cell-killing protein which is a potent poison of bacterial gyrase).

Genetic constructs of the disclosure include a first selectable marker (e.g., beta-lactamase gene for ampicillin resistance) for selection in E. coli for propagation of the genetic construct and a second selectable marker (e.g., aacc gene for gentamicin resistance) for selection in Hs. Genetic constructs of the disclosure also include a counter-selection marker (i.e., suicide gene) such as human herpes simplex virus thymidine kinase (hsvTK) that allows selection against cells that retain the genetic construct backbone after homologous recombination, so as to obtain cells that no longer include the genetic construct backbone in the Hs microbe. In particular embodiments, a genetic construct includes homology arms to enable deletion of a gene in an Hs microbe.

A “recombinant polypeptide” refers to a polypeptide or polyprotein which is not naturally occurring or is made by the artificial combination of two otherwise separated segments of amino acid sequences. This artificial combination may be accomplished by standard techniques of recombinant DNA technology, i.e., a recombinant polypeptide may be encoded by a recombinant polynucleotide. Thus, a recombinant polypeptide is an amino acid sequence encoded by all or a portion of a recombinant polynucleotide.

The term “expression cassette” includes a polynucleotide construct that is generated recombinantly or synthetically and includes regulatory sequences operably linked to a selected polynucleotide to facilitate expression of the selected polynucleotide in a microbe. For example, the regulatory sequences can facilitate transcription of the selected polynucleotide in a microbe, or transcription and translation of the selected polynucleotide in a microbe. Expression of a gene encoding a polypeptide may be upregulated or downregulated by introducing genetic elements such as transcription enhancers or repressors, or translation enhancers or repressors (e.g., modified ribosome binding sites, degradation tags, modified Kozak sequences).

The term “overexpression” refers to a greater expression of a gene encoding a given polypeptide in a genetically modified microbe as compared to a reference expression (e.g., expression in a wild type microbe at any developmental or temporal stage for the gene). In particular embodiments, overexpression can occur when the gene is under the control of a strong promoter (e.g., the P65 promoter). Overexpression may also occur under the control of an inducible promoter. In particular embodiments, overexpression may occur in a microbe where endogenous expression of a given polypeptide normally occurs, but such normal expression is at a lower level. In particular embodiments, overexpression may also occur in a microbe lacking expression of a given polypeptide. Overexpression thus results in a greater than normal production or “overproduction” of a given polypeptide in a microbe. For example, in an Hs microbe where the endogenous nifA promoter has been replaced by a strong constitutive promoter (e.g., P65), the endogenous nifA gene is overexpressed as compared to the endogenous nifA gene operably linked to its native nifA promoter. Overexpression of a gene or polypeptide encoded by the gene may include a 10%, 15%, 20%, 25%, 30%, 35%, 40%, 45%, 50%, 55%, 60%, 65%, 70%, 75%, 80%, 85%, 90%, 95%, 100%, 150%, 200%, or more increase in expression as compared to a reference expression. A reference expression can include: expression of the corresponding endogenous gene or polypeptide encoded by the gene in a wild type microbe or in a microbe that does not contain a genetic modification that leads to expression or overexpression of the gene or polypeptide encoded by the gene; expression of the corresponding endogenous gene operably linked to its endogenous promoter; expression of the corresponding endogenous gene operably linked to a corresponding inducible promoter that has been repressed; expression of the polypeptide encoded by the corresponding endogenous gene operably linked to its endogenous promoter; or other reference expression known to one of skill in the art and appropriate for the experiments being conducted. A gene that has greater expression compared to a reference expression may be referred to as being “upregulated”, as described herein.

The term “downregulation” refers to a lower expression or no expression of a gene encoding a given polypeptide as compared to a reference expression (e.g., expression of a gene in a wild type microbe at any developmental or temporal stage for the gene). Downregulation of a gene in a microbe may occur when the gene is deleted (i.e., knocked out) or partially deleted, when mutations are introduced into the gene or into the promoter operably linked to the gene such that expression of the gene is reduced or eliminated, when elements are introduced upstream of, downstream of, or in a gene to decrease its transcription or translation, or other modifications described herein that may reduce or prevent expression of a gene. For example, glnK expression can be downregulated in an Hs microbe by genetically modifying the Hs microbe to delete the endogenous glnK gene. Downregulation may also occur when the gene is under the control of an inducible promoter, as expression of a gene from an inducible promoter can be reduced or prevented by inhibiting the activity of the inducible promoter, for example, by expressing or providing an inhibitor molecule that binds to the inducible promoter such that the inducible promoter is not able to drive expression of the gene. Downregulation may be in the context of reducing or preventing expression and/or activity of a polypeptide encoded by a gene. For example, GS expression and/or activity can be downregulated in an Hs microbe by operably linking endogenous glnA gene to a heterologous promoter that drives weaker expression of glnA as compared to endogenous Hs glnA gene promoter, by operably linking glnA gene to an RBS modified to decrease translation efficiency of the endogenous glnA gene as compared to the endogenous glnA gene operably linked to the corresponding endogenous unmodified RBS, or by operably linking glnA gene to a degradation tag. In particular embodiments, downregulation of a first protein activity may include modifying expression and/or activity of a second protein expression and/or activity that regulates the first protein expression and/or activity. For example, GS expression and/or activity can be downregulated in an Hs microbe by truncating an N-terminal region of the glnE adenylyl transferase such that glnE is unable to de-adenylate GS, keeping GS inactive. Downregulating includes decreasing, reducing, inhibiting, ameliorating, diminishing, lessening, blocking, or preventing, with respect to expression of a gene, expression of a polypeptide, activity of a polypeptide, or expression and/or activity of any genetic elements that may lead to decreasing expression of a gene or polypeptide. Downregulation of a gene or polypeptide encoded by a gene may be achieved by any means described herein and may lead to a 10%, 20%, 30%, 40%, 50%, 60%, 70%, 80%, 90%, 91%, 92%, 93%, 94%, 95%, 96%, 97%, 98%, 99%, or 100% reduction in gene expression or polypeptide expression and/or activity as compared to a reference expression or activity. A reference expression can include: expression of the corresponding endogenous gene or polypeptide encoded by the gene in a wild type microbe or in a microbe that does not contain a genetic modification that leads to the downregulation; expression of the corresponding endogenous gene operably linked to its endogenous promoter; expression of the corresponding endogenous gene operably linked to a corresponding inducible promoter that has been induced; or other reference condition known to one of skill in the art and appropriate for the experiments being conducted.

On the other hand, “upregulation” refers to a higher expression of a gene encoding a given polypeptide as compared to a reference expression (e.g., expression of a gene in a wild type microbe at any developmental or temporal stage for the gene). Upregulation may occur when: a heterologous gene is introduced into a microbe (e.g., integrated into the genome of a microbe) as part of a genetic construct including a promoter operably linked to the heterologous gene; a heterologous promoter is integrated upstream of an endogenous gene such that the heterologous promoter is operably linked to the endogenous gene and replaces the endogenous promoter of the endogenous gene, and the heterologous promoter drives greater expression of the gene as compared to the endogenous promoter; elements are introduced upstream of, downstream of, or in a gene to increase its transcription or translation; or other modifications described herein that may increase expression of a gene. For example, nifA expression can be upregulated in an Hs microbe by integrating a genetic construct including a heterologous nifA gene operably linked to a heterologous promoter into the genome of the Hs microbe or by integrating a heterologous promoter upstream of the endogenous nifA gene such that the heterologous promoter is operably linked to the endogenous nifA gene and replaces the endogenous nifA promoter, and the heterologous promoter drives greater expression of the nifA gene as compared to the endogenous nifA promoter. Upregulation may also occur when the gene is under the control of an inducible promoter, as expression of a gene from an inducible promoter can be increased by inducing the activity of the inducible promoter, for example, by removing an inhibitor molecule that is repressing the inducible promoter or by providing a molecule that induces activity of the inducible promoter such that the inducible promoter drives expression of the gene. Upregulation may be in the context of increasing expression and/or activity of a polypeptide encoded by a gene. Upregulation includes elicitation, initiation, increasing, augmenting, boosting, improving, enhancing, amplifying, promoting, or providing, with respect to expression of a gene, expression of a polypeptide, activity of a polypeptide, or expression and/or activity of any genetic elements that may lead to expression of a gene or polypeptide. Upregulation of a gene or polypeptide encoded by a gene may be achieved by any means described herein and may lead to a 10%, 15%, 20%, 25%, 30%, 35%, 40%, 45%, 50%, 55%, 60%, 65%, 70%, 75%, 80%, 85%, 90%, 95%, 100%, 150%, 200%, or more increase in gene expression or polypeptide expression and/or activity as compared to a reference expression or activity. A reference expression can include: expression of the corresponding endogenous gene or polypeptide encoded by the gene in a wild type microbe or in a microbe that does not contain a genetic modification that leads to the upregulation; expression of the corresponding endogenous gene operably linked to its endogenous promoter; expression of the corresponding endogenous gene operably linked to a corresponding inducible promoter that has been repressed; or other reference expression known to one of skill in the art and appropriate for the experiments being conducted.

In particular embodiments, a genetically modified microbe includes a nucleic acid (e.g., a gene) where expression of the gene is regulated by a promoter and/or regulatory elements. A promoter and/or regulatory elements are often introduced at a suitable location relative to a gene of interest. For example, a promoter (e.g., a constitutive or an inducible promoter) is often placed 5′ of a transcription start site of a gene of interest. In particular embodiments, a nucleic acid includes a promoter and/or regulatory elements necessary to drive the expression of a gene (e.g., a heterologous gene or an endogenous gene). A promoter can be an endogenous promoter, a heterologous promoter, or a combination thereof. In particular embodiments, a promoter is a constitutive promoter. In particular embodiments, a constitutive promoter includes a T7 promoter, an SP6 promoter, a T3 promoter, a P65 promoter (Johns et al. Nature Methods 2018; 15:323-329), a CP25 promoter from Borrelia burgdorferi (Stevenson et al. J Bacteriol 1997; 179(13):4285-4291), a CP32 promoter from Borrelia burgdorferi (Stevenson et al. J Bacteriol 1997; 179(13):4285-4291), or any suitable constitutive promoter. In particular embodiments, a constitutive promoter includes a P65 promoter. In particular embodiments, a constitutive promoter includes a CP25 promoter. In particular embodiments, a constitutive promoter includes a CP32 promoter.

In particular embodiments, a microbe is genetically engineered to include a gene under the control of an inducible promoter. An inducible promoter is often a nucleic acid sequence that directs the conditional expression of a gene. An inducible promoter can be an endogenous promoter, a heterologous promoter, or a combination thereof. An inducible promoter can include an operon system. In particular embodiments, an inducible promoter requires the presence of a certain compound, nutrient, amino acid, sugar, peptide, protein or condition (e.g., light, oxygen, heat, cold) to induce gene activity (e.g., transcription). In particular embodiments, an inducible promoter includes one or more repressor elements. In particular embodiments, an inducible promoter including a repressor element requires the absence of a certain compound, nutrient, amino acid, sugar, peptide, protein or condition to induce gene activity (e.g., transcription). Any suitable inducible promoter, system, or operon can be used to regulate the expression of a gene. Non-limiting examples of inducible promoters include temperature inducible promoters (e.g., heat inducible PgroES promoter, heat inducible phage lambda pL promoter, heat inducible phage lambda pR promoter, cold inducible espA promoter), lactose regulated systems (e.g., lactose operon systems), sugar regulated systems, metal regulated systems, steroid regulated systems, alcohol regulated systems, IPTG inducible systems (e.g., pLac promoter), arabinose regulated systems (e.g., arabinose operon systems, pBad promoter), synthetic amino acid regulated systems (e.g., see Rovner et al. (2015) Nature 518(7537):89-93), fructose repressors, a tac promoter/operator (pTac), tryptophan promoters (e.g., Ptrp, induced by tryptophan depletion or by addition of β-indoleacrylic acid), alkaline phosphatase promoters (e.g., PhoA promoter induced by phosphate limitation), recA promoters (e.g., recA promoter induced by UV light), proU promoters (e.g., osmotically inducible proU promoter), cst promoters (e.g., cst promoter inducible by carbon starvation), tetA promoters (e.g., tetracycline inducible tetA promoter), cadA and cadR promoters (e.g., PcadA and PcadR induced by cadmium), nar promoters (e.g., nar promoter induced by oxygen), or combinations thereof.

In particular embodiments, expression of a gene can be controlled in additional ways known to one of skill in the art including modifying: gene copy number, number of copies of transcription factors binding the promoter operably linked to the gene; transcription factor binding to the gene promoter; RNA polymerase binding affinity for the gene promoter; ribosome binding affinity for the RBS; mRNA decay rate; and protein decay rate (Brewster et al. (2012) PLoS Comput Biol 8(12): e1002811). In particular embodiments, a promoter such as T7 can be regulated using a system with a temperature sensitive intein inserted in the protein sequence of T7 RNA polymerase (Korvin and Yadav (2018) Molecular Systems Design & Engineering 3(3):550-559). The polymerase is only active and able to drive gene expression when the intein is spliced out at the appropriate temperature.

The term “operably linked” refers to polynucleotide sequences or amino acid sequences placed into a functional relationship with one another. For instance, a promoter or enhancer is operably linked to a coding or non-coding sequence if it regulates, or contributes to the modulation of, the transcription of the coding or non-coding sequence. In particular embodiments, regulatory sequences operably linked to a coding sequence are typically contiguous to the coding sequence. However, enhancers can function when separated from a promoter by up to several kilobases or more. Accordingly, some polynucleotide elements may be operably linked but not contiguous. In particular embodiments, a heterologous promoter or heterologous regulatory elements include promoters and regulatory elements that are not normally associated with a particular nucleic acid in nature.

A termination region may be provided by the naturally occurring or endogenous transcriptional termination region of the polynucleotide sequence encoding a protein of the disclosure. Alternatively, the termination region may be derived from a different source. For the most part, the source of the termination region is generally not considered to be critical to the expression of a recombinant protein and a wide variety of termination regions can be employed without adversely affecting expression.

In particular embodiments, a genetic construct of the disclosure can be propagated in vitro in a host cell suitable for replication of the genetic construct. Host cells can include bacterial cells, mammalian cells, yeast cells, insect cells, or plant cells. In particular embodiments, the host cell is a bacterium, e.g., E. coli. The selection of an appropriate host is deemed to be within the scope of those skilled in the art. A recombinant host cell includes a host cell into which a genetic construct has been introduced. In particular embodiments, a host cell including a genetic construct is conjugated with an Hs microbe to transfer the genetic construct to the Hs microbe genome by homologous combination. An exemplary high-throughput protocol for conjugation of E. coli and Hs is described in Example 1.

In particular embodiments, a genetic construct is introduced into a microbe and/or a microbe is transformed with a genetic construct using a suitable technique. Non-limiting examples of suitable techniques for introducing a nucleic acid into a microbe include conjugation, electroporation, transduction (e.g., injection of a nucleic acid by a bacteriophage), microinjection, by inducing competence (e.g., by addition of alkali cations, cesium, lithium, polyethylene glycol or by osmotic shock), or combinations thereof. In particular embodiments, a genetic construct is introduced into a microbe using conjugation. In particular embodiments, transformed Hs microbes are selected for integration of a nucleic acid into the Hs genome by using a suitable selection method (e.g., a selection marker such as an antibiotic marker (e.g., aacc gene for gentamicin resistance)) and/or counter selection method. In particular embodiments, a counter selection marker includes herpes simplex virus thymidine kinase (hsvTK) that can be used to select against cells expressing hsvTK (and thus selecting against cells containing the genetic construct backbone) by growing the microbe in the nucleoside analog dP.

As would be appreciated by one of ordinary skill in the art, the genetic engineering strategies described herein to increase atmospheric nitrogen fixation in a microorganism may also be applied to one or more of the following microorganisms: Proteobacteria (e.g., Pseudomonas, Enterobacter, Slenotrophononas, Burkholderia, Rhizobium, Herbaspirillum, Pantoea, Serratia, Rahnella, Azospirillum, Azorhizobium, Azotobacter, Duganella, Delftia, Bradyrhizobium, Sinorhizobium and Halomonas), Firmicutes (e.g., Bacillus, Paenibacillus, Lactobacillus. Mycoplasma, and Acetobacterium), and Actinobacteria (such as Streptomyces, Rhodococcus, Microbacterium, and Curtobacterium). In some embodiments, a microorganism from one or more of the following taxa may be genetically modified as described herein: Achromobacter, Acidithiobacillus, Acidovorax, Acinetobacter, Actinoplanes, Adlercreutzia, Aerococcus, Aeromonas, Afipia, Agromyces, Ancylobacter, Arthrobacter, Atopostipes, Azospirillum, Bacillus, Bdellovibrio, Beijerinckia, Bosea, Bradyrhizobium, Brevibacillus, Brevundimonas, Burkholderia, Candidatus, Caulobacter, Cellulomonas, Cellvibrio, Chryseobacterium, Citrobacter, Clostridium, Coraliomargarita, Corynebacterium, Cupriavidus, Curtobacterium, Curvibacter, Deinococcus, Delftia, Desemzia, Devosia, Dokdonella, Dyella, Enhydrobacter, Enterobacter, Enterococcus, Erwinia, Escherichia, Escherichia/Shigella, Exiguobacterium, Ferroglobus, Filimonas, Finegoldia, Flavisolibacter, Flavobacterium, Frigoribacterium, Gluconacetobacter, Hafnia, Halobaculum, Halomonas, Halosimplex, Herbaspirillum, Hymenobacter, Klebsiella, Kocuria, Kosakonia, Lactobacillus, Leclercia, Lentzea, Luteibacter, Luteimonas, Massilia, Mesorhizobium, Methylobacterium, Microbacterium, Micrococcus, Microvirga, Mycobacterium, Neisseria, Nocardia, Oceanibaculum, Ochrobactrum, Oribacterium, Oligotropha, Oxalobacter, Paenibacillus, Pantoea, Pelonionas, Perlucidibaca, Plantibacter, Polynucleobacter, Propionibacterium, Propionibacterium, Pseudoclavibacter, Pseudomonas, Pseudonocardia, Pseudoxanthomonas, Psychrobacter, Rahnella, Ralstonia, Rheinheimera, Rhizobium, Rhodococcus, Rhodopseudomonas, Roseateles, Ruminococcus, Sebaldella, Sediminibacterium, Serratia, Shigella, Shinella, Sinorhizobium, Sinosporangium, Sphingobacterium, Sphingomonas, Sphingopyxis, Sphingosinicella, Staphylococcus, Stenotrophomonas, Streptococcus, Streptomyces, Stygiolobus, Sulfurisphaera, Tatumella, Tepidimonas, Thermomonas, Thiobacillus, Variovorax, Xanthomonas, and Zimmermannella. In addition, the genetic engineering strategies described herein to increase atmospheric nitrogen fixation in a microorganism may include genetic material from microorganisms from one of the above-referenced genera. In a particular embodiment, the heterologous NifA gene introduced into the AmtB gene is from a different genus than Herbaspirillum, including, for example, any of the taxa listed above.

### (ii) Assays

In vitro assays can be performed to assess whether a genetically modified Hs microbe of the disclosure has increased nitrogen fixation as compared to a control Hs microbe. In particular embodiments, a control Hs microbe includes a non-genetically modified Hs microbe. In particular embodiments, a non-genetically modified Hs microbe includes Hs strain Z78 (ATCC 35893). In particular embodiments, a non-genetically modified Hs microbe includes Hs strain SmR1 (a spontaneous streptomycin resistant mutant of strain Z78 (Baldani et al. (1986) Int J Syst Bacteriol 36: 86-93). In particular embodiments, a control Hs microbe includes an Hs microbe with increased nitrogen fixation as compared to a non-genetically modified Hs microbe. In particular embodiments, a negative control Hs microbe includes an Hs microbe that is the same as a non-genetically modified Hs microbe but has a deletion of the nifA gene. In particular embodiments, a control Hs microbe includes an Hs microbe that is the same as a non-genetically modified Hs microbe but has a deletion of the nifH gene.

In particular embodiments, nitrogenase activity can be measured directly in a genetically modified Hs microbe of the disclosure by an ¹⁵N dilution assay. An ¹⁵N dilution assay measures incorporation of fixed nitrogen into amino acids. Cells are initially grown on rich media containing a ¹⁵N enriched nitrogen source. As the cells grow in this media, they will fix and incorporate ¹⁵N in their biomass. After reaching a target biomass, cells are transferred to a nitrogen free media under regular atmosphere. As the cells are growing, they will incorporate atmospheric ¹⁴N into their biomass, thus diluting the initial pool of ¹⁵N. Arginine is used as a marker and the ratio of ¹⁴NArg/¹⁵NArg is obtained. In particular embodiments, nitrogenase activity of a genetically modified Hs microbe is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to nitrogenase activity of an unmodified Hs microbe as measured by an ¹⁵N dilution assay.

In particular embodiments, nitrogenase activity can be measured directly in a genetically modified Hs microbe of the disclosure by an ¹⁵N₂ fixing assay. In particular embodiments, an ¹⁵N₂ fixing assay includes adding ¹⁵N₂ gas directly as a bubble to water. Rates of N₂ fixation can then be calculated from the incorporation of ¹⁵N₂ gas into biomass (Montoya et al. (1996) Appl. Environ. Microbiol. 62:986-993). In particular embodiments, an ¹⁵N₂ fixing assay is performed when the rates of nitrogen fixation are high and high sensitivity is not necessary. In particular embodiments, nitrogenase activity of a genetically modified Hs microbe is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to nitrogenase activity of an unmodified Hs microbe as measured by an ¹⁵N₂ fixing assay.

In particular embodiments, nitrogenase activity can be measured in a genetically modified Hs microbe of the disclosure by an assay that measures the conversion of N₂ to NH₄⁺. For example NH₄⁺ production can be measured via analytical methods, such as chromatography, known in the art. In particular embodiments, nitrogenase activity of a genetically modified Hs microbe is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to nitrogenase activity of an unmodified Hs microbe as measured by an assay that measures the conversion of N₂ to NH₄. In particular embodiments, nitrogenase activity of a genetically modified Hs microbe is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to nitrogenase activity of an unmodified Hs microbe as measured by an assay known to one of skill in the art.

In particular embodiments, nitrogenase activity can be measured indirectly in a genetically modified Hs microbe of the disclosure by a carbon, hydrogen, and nitrogen analyzer (CHN analyzer), which includes flash combustion of a sample to cause an instantaneous oxidization into simple compounds which are then detected with thermal conductivity detection or infrared spectroscopy. In particular embodiments, the CHN analyzer can be used when rates of nitrogen fixation are very high. In particular embodiments, nitrogenase activity of a genetically modified Hs microbe is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to nitrogenase activity of an unmodified Hs microbe as measured by a CHN analyzer.

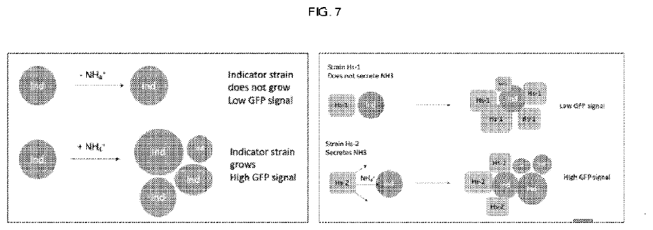

In particular embodiments, an ammonia biosensor indicator assay can be used to measure ammonia secretion from a genetically modified Hs microbe of the disclosure. This assay includes an Hs “indicator” strain that is tagged with a detectable reporter (e.g., green fluorescent protein) which is operably linked to a constitutive promoter and modified to be an NH₃ auxotroph. The ammonia produced by a test strain (for example, a genetically engineered strain of the present disclosure) will diffuse through the membrane into the broth and will be assimilated by the Hs indicator strain. As the Hs indicator strain grows, the signal from the detectable reporter (e.g., fluorescence) will also increase. The fluorescence measurement of this indicator strain can be used as an estimate of extracellular ammonia concentration when mixed with a strain of interest. Particular embodiments provide for an assay including an Hs microbe including replacement of the endogenous nifH gene with a detectable reporter expression cassette including a constitutive promoter. The detectable reporter expression cassette can include any fluorescent protein that can be visualized and quantified by a fluorescence detector. In particular embodiments, the detectable reporter includes green fluorescent proteins, red fluorescent proteins, blue fluorescent proteins, and yellow fluorescent proteins. With reference to FIG. 7, the indicator strain does not multiply in the absence of ammonium and exhibits a low signal from green fluorescent protein (GFP). In the presence of ammonium, the indicator strain multiplies and exhibits a high signal from GFP per whole culture. Ammonia and ammonium are used interchangeably. Thus, in an indicator (ammonia biosensor) assay, an experimental strain can be tested for its ability to secrete ammonia by mixing it with the indicator strain. If an experimental strain does not secrete ammonia (strain Hs-1 in FIG. 7), the indicator strain will not multiply and low GFP signal is detected. If an experimental strain does secrete ammonia (strain Hs-2 in FIG. 7), the indicator strain will multiply and high GFP signal is detected. The indicator assay is conducted using control and experimental strains. Control strains include: a high biological positive Hs strain known to secrete ammonia; a wild type Hs strain; a ΔnifA negative Hs control; and the indicator strain. In particular embodiments, a high biological positive gives a fluorescence signal that is 40× above that of the wild type strain. In particular embodiments, ammonium secretion from the genetically modified Hs microbe is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to ammonium secretion of a control Hs microbe as measured by a biosensor indicator assay. In particular embodiments, the control Hs microbe is a non-genetically modified Hs microbe. In particular embodiments, the control Hs microbe is a Hs microbe genetically modified to increase nitrogen fixation.

Measurements of plant agronomic characteristics can be measured to assess the effects of association of a genetically modified Hs microbe of the disclosure with an agricultural plant. Nitrogen (N) is an essential element for plant growth. N fertilizer application significantly enhances plant growth, biomass accumulation, and grain yield in plants. In some embodiments, final grain yield is highly correlated to early plant growth including biomass accumulation. These metrics are known to correlate to applied nitrogen conditions in a dose dependent manner under greenhouse and field conditions. In particular embodiments, early plant responses to N application applied in a greenhouse translates to plant growth and yield responses to N fertilizer application responses in the field.

In some embodiments, in planta assays can be used to screen and characterize engineered microbes in the greenhouse. Plant seedlings inoculated with a genetically engineered microbe of the disclosure can be fertigated with a solution with different amounts of N. For example, inoculated plants can be fertigated with 0 ppm N, 5 ppm N, 10 ppm N, 15 ppm N, 20 ppm N, 25 ppm N, or 100 ppm in appropriate containers for a period of time (e.g., 2 weeks, 4 weeks, 6 weeks, 8 weeks, etc.). To estimate the amount of N supplemented by a genetically engineered microbe of the disclosure when it is applied to a plant, plant characteristics including plant height, leaf number, leaf greenness, plant biomass (e.g., shoot fresh weight and/or shoot dry weight) can be measured and compared to a control that is fertigated with the same regime. In particular embodiments, a control includes a plant or a population of plants of the same genus and species that has not been inoculated with the genetically engineered microbe. In particular embodiments, a control includes a plant or a population of plants of the same genus and species that has been inoculated with a corresponding microbe (i.e., microbe of the same genus and species) that has not been genetically engineered to increase nitrogen fixation. In particular embodiments, a control includes a plant or a population of plants of the same variety or cultivar. Plant characteristics can be evaluated weekly or at a particular time after planting. In particular embodiments, the plant is corn.

In particular embodiments, growth stage can be assessed as leaf number, which is recorded weekly for each plant by counting fully expanded leaves. In particular embodiments, plant height can be measured weekly from soil surface to the tallest extended leaf tip (manually straightened up).

In particular embodiments, leaf greenness is an indicator of plant N and can be measured by a Soil Plant Analysis Development (SPAD, Minolta Camera Co., Osaka, Japan) chlorophyll meter. SPAD measurements provide a quick and non-destructive method that enables users to measure chlorophyll content in the field. In particular embodiments, SPAD is useful to determine in situ nitrogen (N) status (Arregui et al. (2006) Eur. J. Agron. 24:140-148; Ziadi et al. (2008) Agron. J. 100:1264-1273; Yuan et al. (2016) Field Crops Res. 185:12-20). The SPAD meter measures the difference between the transmittance of a red (650 nm) and an infrared (940 nm) light through the leaf, generating a three-digit SPAD value (Uddling et al. (2007) Photosynth. Res. 91:37-46). An exemplary protocol includes the following. One day before harvest, leaf greenness is measured on the upmost fully expanded leaf by using a SPAD meter. Four readings are taken in the middle portion of the leaf and the average of the 4 readings for each plant is recorded.

In particular embodiments, shoot fresh weight can be measured as follows. On day 28 after planting, all plants are watered early in the morning to make sure the soil in every pot is not dried to have uniform plant water content across the experiment. Plants are watered again early afternoon if the harvest is not completed in the morning. A plant is cut from the soil surface and shoot fresh weight measured immediately and placed into a paper bag with a plant tag on the bag for drying. All plants are cut uniformly from soil surface across the entire experiment.

In particular embodiments, shoot dry weight can be measured as follows. Bags with plants are placed in the oven in the growth chamber room at 105° C. temperature for 15 min to stop all biological activities. Then the plant samples are dried with oven temperature at 75° C. until constant weight (weigh 10 bags from different positions in the oven with less than 1% weight decrease over 24 hours). Shoot dry weight (0% moisture) will be taken immediately (within 30 seconds after the bag is removed from oven).

In some embodiments, an in planta assay in a greenhouse can include the following. Inoculated corn seedlings are fertigated with 25 ppm nitrogen (N) (plus 0 ppm and 100 ppm for characterization assays) in four inch pots for four weeks. Plant growth is evaluated weekly by measuring plant height and leaf number. Leaf greenness is measured by a SPAD meter. Plant biomass (shoot fresh weight and dry weight) is evaluated on day 28 after planting. To estimate the amount of N supplemented by the strains applied, plant biomass is compared between engineered microbe treated plants and non-engineered microbe treated (wild type) plants and supplied N was inferred from a standard curve with chemical fertilizer.

### (iii) Compositions and Formulations

The genetically modified Hs microbe described herein is intended to be useful in the improvement of agricultural plants, and as such, may be formulated with other compositions as part of an agriculturally compatible carrier. The carrier composition including the genetically modified Hs microbe may be prepared for agricultural application as a liquid, a solid, or a gas formulation. In particular embodiments, the carrier composition includes a vehicle to associate the genetically modified Hs microbe with an agricultural plant part. Suitable formulations that may be prepared include wettable powders, granules, gels, agar strips or pellets, thickeners, biopolymers, microencapsulated particles; aqueous flowables; aqueous suspensions; and water-in-oil emulsions.

In particular embodiments, a formulation can include: a buffer, a tackifier, a microbial stabilizer, a surfactant, an adherent, a fungicide, an herbicide, a nematicide, an insecticide, a virucide, a plant growth regulator, a rodenticide, a desiccant, a nutrient, or combinations thereof.

The carrier can be a solid carrier or liquid carrier, and in various forms including microspheres, powders, and emulsions. In particular embodiments, the agricultural carrier may be soil or a plant growth medium. The carrier may be any one or more of a number of carriers that confer a variety of properties, such as increased stability, wettability, or dispersibility. Wetting agents such as natural or synthetic surfactants, which can be nonionic or ionic surfactants, or a combination thereof, can be included in a composition of the disclosure.

Solid compositions can be prepared by dispersing the genetically modified Hs microbe of the disclosure in and on an appropriately divided solid carrier, including: loam, sand, kaolin clay, talc, pyrophyllite, bentonite, montmorillonite, diatomaceous earth, fuller's earth, acid white soil, pasteurized soil, vermiculite, and pearlite; and inorganic salts such as ammonium sulfate, ammonium phosphate, ammonium nitrate, urea, ammonium chloride, and calcium carbonate. Also, organic fine powders such as wheat flour, wheat bran, and rice bran may be used. Mixtures of any of the aforementioned ingredients are also included as carriers, such as pesta (flour and kaolin clay), or agar or flour-based pellets in loam, sand, or clay. When such formulations are used as wettable powders, biologically compatible dispersing agents such as non-ionic, anionic, amphoteric, or cationic dispersing and emulsifying agents can be used.

In particular embodiments, a genetically modified Hs microbe of the present disclosure can be mixed or suspended in water or in aqueous solutions. Suitable liquid diluents or carriers include water, aqueous solutions, petroleum distillates, vegetable oils such as soybean oil and cottonseed oil, glycerol, ethylene glycol, polyethylene glycol, propylene glycol, polypropylene glycol, or other liquid carriers. In particular embodiments, a liquid carrier can include a pH ranging from 5-9. In particular embodiments, a liquid carrier has a pH of 7. Water-in-oil emulsions can also be used to formulate a composition that includes the genetically modified Hs microbe (see, for example, U.S. Pat. No. 7,485,451). Formulations may include food sources for the cultured genetically modified Hs microbe, such as barley, rice, or other biological materials such as seed, plant parts, sugar cane bagasse, hulls or stalks from grain processing, ground plant material or wood from building site refuse, sawdust or small fibers from recycling of paper, fabric, or wood. Other suitable formulations will be known to those skilled in the art.

In particular embodiments, the formulation can include a tackifier, sticker, or adherent. Such agents are useful for combining the genetically modified Hs microbe disclosed herein with carriers that can contain other compounds (e.g., control agents that are not biologic) to yield a coating composition. Such compositions help create coatings around the plant or plant part to maintain contact between the genetically modified Hs microbe and other agents with the plant or plant part. In particular embodiments, adherents (stickers, or tackifiers) include: alginate, gums, starches, lecithins, formononetin, polyvinyl alcohol, alkali formononctinate, hesperetin, polyvinyl acetate, cephalins, gum arabic, xanthan gum, carragennan, polyglutamic acid (PGA), other biopolymers, mineral oil, polyethylene glycol (PEG), polyvinyl pyrrolidone (PVP), arabino-galactan, methyl cellulose, PEG 400, chitosan, polyacrylamide, polyacrylate, polyacrylonitrile, glycerol, triethylene glycol, vinyl acetate, gellan gum, polystyrene, polyvinyl, carboxymethyl cellulose, gum ghatti, and polyoxyethylene-polyoxybutylene block copolymers. Other examples of adherent compositions that can be used in the formulation include those described in EP 0818135, CA 1229497, WO 2013/090628, EP 0192342, WO 2008/103422, and CA 1041788.

In particular embodiments, the formulation may include an anti-caking agent.

In particular embodiments, the formulation can include a surfactant, wetting agent, emulsifier, stabilizer, or anti-foaming agent. Examples of surfactants include: nitrogen-surfactant blends such as Prefer 28 (Cenex), Surf-N(US), Inhance (Brandt), P-28 (Wilfarm) and Patrol (Helena); esterified seed oils such as Sun-It II (AmCy), MSO (UAP), Scoil (Agsco), Hasten (Wilfarm) and Mes-100 (Drexel); and organo-silicone surfactants such as Silwet L77 (UAP), Silikin (Terra), Dyne-Amic (Helena), Kinetic (Helena), Sylgard 309 (Wilbur-Ellis) and Century (Precision); polysorbate 20; polysorbate 80; Tween 20; Tween 80; Scattics; Alktest TW20; Canarcel; Peogabsorb 80; Triton X-100; Conco NI; Dowfax 9N; Igebapl CO; Makon; Neutronyx 600; Nonipol NO; Plytergent B; Renex 600; Solar NO; Sterox; Serfonic N; T-DET-N; Tergitol NP; Triton N; IGEPAL CA-630; Nonident P-40; and Pluronic. In particular embodiments, the surfactant is present at a concentration of between 0.01% v/v to 10% v/v. In particular embodiments, the surfactant is present at a concentration of between 0.1% v/v to 1% v/v. An example of an anti-foaming agent would be Antifoam-C.

In particular embodiments, the formulation includes a microbial stabilizer. Such an agent can include a desiccant. A “desiccant” can include any compound or mixture of compounds that can be classified as a desiccant regardless of whether the compound or compounds are used in such concentrations that they in fact have a desiccating effect on the liquid inoculant. Such desiccants are ideally compatible with the genetically modified Hs microbe and should promote the ability of the genetically modified Hs microbe to survive application on the seeds and to survive desiccation. Examples of suitable desiccants include one or more of trehalose, sucrose, glycerol, and methylene glycol. Other suitable desiccants include non-reducing sugars and sugar alcohols (e.g., mannitol or sorbitol). The amount of desiccant introduced into the formulation can range, for example, from 5% to 50% by weight/volume (w/v), between 10% to 40% w/v, between 15% and 35% w/v, or between 20% and 30% w/v.

In particular embodiments, it is advantageous for the formulation to include agents such as a fungicide, an herbicide, a nematicide, an insecticide, a plant growth regulator, a rodenticide, a bactericide, a virucide, a nutrient, or any combination thereof. Such agents are ideally compatible with the agricultural plant part or plant onto which the formulation is applied. In particular embodiments, an agent that is compatible with an agricultural plant part or plant is not deleterious to the growth or health of the plant part or plant. In particular embodiments, the agent is ideally one which does not cause safety concerns for human, animal or industrial use (e.g., no safety issues, or the compound is sufficiently labile that the commodity plant product derived from the plant contains negligible amounts of the compound). In particular embodiments, an herbicide includes: imazethapyr, 2,2-dichloropropionic acid, glyphosate, 2,4-dichlorophenoxyacetic acid (2,4-D), and derivatives thereof. In particular embodiments, formulations of the disclosure include a pesticide. In particular embodiments, a pesticide includes: O, S-dimethyl acetylphos-phoramidothioate (acephate), carbamate, carbaryl, chrlopyrifos-methyl, dicrotophos, indoxacarb, 2-(dimethoxyphosphinothioylthio) (malathion), methomyl, methoxyfenozide, methyl parathion, pyrethrins, synthetic pyrethroids (such as bifenthrin, cypermethrin and the like), pyrethroids, protenophos, phorate, spinosyn, dimethyl N, N′-[thiobis[(methylimino)carbonyloxy]]—bis[ethanimidothioate](thiodicarb), and derivatives thereof.

Nutrient additives to the formulation may include fertilizer compositions such as nitrogen, phosphorous, or potassium.

In particular embodiments, the formulation is suited for coating of a genetically modified Hs microbe onto plant parts. The genetically modified Hs microbe described in the present disclosure can confer many fitness benefits to a host plant. The ability to confer such benefits by coating the genetically modified Hs microbe on the surface of plant parts has many potential advantages, particularly when used in a commercial (agricultural) scale. In particular embodiments, the present disclosure provides a composition including a plant or plant part and a genetically modified Hs microbe described herein. In particular embodiments, the plant part is a seed and the genetically modified Hs microbe is part of a seed coating.

The genetically modified Hs microbe described herein can be combined with one or more of the agents described above to yield a formulation suitable for combining with an agricultural plant part or plant. A genetically modified Hs microbe of the disclosure can be obtained from growth in culture, for example, using a synthetic growth medium. In addition, a genetically modified Hs microbe disclosed herein can be cultured on solid media, for example on petri dishes, scraped off and suspended into a formulation. A genetically modified Hs microbe at different growth phases can be used. For example, a genetically modified Hs microbe at lag phase, early-log phase, mid-log phase, late-log phase, stationary phase, early death phase, or death phase can be used.

The formulations can include a genetically modified Hs microbe disclosed herein that is between 0.1% and 90% wet weight, between 3% and 75% wet weight, between 5% and 60% wet weight, or between 10% and 50% in wet weight. In particular embodiments, a formulation includes a genetically modified Hs microbe disclosed herein that is 1% wet weight, 2% wet weight, 3% wet weight, 4% wet weight, 5% wet weight, 6% wet weight, 7% wet weight, 8% wet weight, 9% wet weight, 10% wet weight, 11% wet weight, 12% wet weight, 13% wet weight, 14% wet weight, 15% wet weight, 16% wet weight, 17% wet weight, 18% wet weight, 19% wet weight, 20% wet weight, 21% wet weight, 22% wet weight, 23% wet weight, 24% wet weight, 25% wet weight, 26% wet weight, 27% wet weight, 28% wet weight, 29% wet weight, 30% wet weight, 31% wet weight, 32% wet weight, 33% wet weight, 34% wet weight, 35% wet weight, 36% wet weight, 37% wet weight, 38% wet weight, 39% wet weight, 40% wet weight, 41% wet weight, 42% wet weight, 43% wet weight, 44% wet weight, 45% wet weight, 46% wet weight, 47% wet weight, 48% wet weight, 49% wet weight, 50% wet weight, 51% wet weight, 52% wet weight, 53% wet weight, 54% wet weight, 55% wet weight, 56% wet weight, 57% wet weight, 58% wet weight, 59% wet weight, 60% wet weight, 61% wet weight, 62% wet weight, 63% wet weight, 64% wet weight, 65% wet weight, 66% wet weight, 67% wet weight, 68% wet weight, 69% wet weight, 70% wet weight, 71% wet weight, 72% wet weight, 73% wet weight, 74% wet weight. 75% wet weight, 76% wet weight, 77% wet weight, 78% wet weight, 79% wet weight, 80% wet weight, 81% wet weight, 82% wet weight, 83% wet weight, 84% wet weight, 85% wet weight, 86% wet weight, 87% wet weight, 88% wet weight, 89% wet weight, 90% wet weight, or more of the formulation.

The concentration of the genetically modified Hs microbe in a carrier may depend upon the carrier. However, any concentration that will achieve plant-enhancing characteristics is desired. In particular embodiments, the formulation includes at least 10² colony forming unit (CFU) genetically modified Hs microbe per mL of liquid formulation, between 10² and 10³ CFU per mL, at least 10³ CFU per mL, between 10³ and 10⁴ CFU per mL, at least 10⁴ CFU per mL, between 10⁴ and 10⁵ CFU per mL, at least 10⁵ CFU per mL, between 10⁵ and 10⁶ CFU per mL, at least 10⁶ CFU per mL, between 10⁶ and 10⁷ CFU per mL, at least 10⁷ CFU per mL, between 10⁷ and 10⁸ CFU per mL, at least 10⁸ CFU per mL, between 10⁸ and 10⁹ CFU per mL, at least 10⁹ CFU per mL, between 10⁹ and 10¹⁰ CFU per mL, at least 10¹⁰ CFU per mL, between 10¹⁰ and 10¹¹ CFU per mL, at least 10¹¹ CFU per mL, or greater than 10¹¹ CFU genetically modified Hs microbe per mL of liquid formulation.

In particular embodiments, the formulation includes at least 10² CFU genetically modified Hs microbe per gram of non-liquid formulation, between 10² and 10³ CFU per gram, at least 10³ CFU per gram, between 10³ and 10⁴ CFU per gram, at least 10⁴ CFU per gram, between 10⁴ and 10⁵ CFU per gram, at least 10⁵ CFU per gram, between 10⁵ and 10⁶ CFU per gram, at least 10⁶ CFU per gram, between 10⁶ and 10⁷ CFU per gram, at least 10⁷ CFU per gram, between 10⁷ and 10⁸ CFU per gram, at least 10⁸ CFU per gram, between 10⁸ and 10⁹ CFU per gram, at least 10⁹ CFU per gram, between 10⁹ and 10¹⁰ CFU per gram, at least 10¹⁰ CFU per gram, between 10¹⁰ and 10¹¹ CFU per gram, at least 10¹¹ CFU per gram, or greater than 10¹¹ CFU genetically modified Hs microbe per gram of non-liquid formulation.

In particular embodiments, the formulation is applied to a plant or plant part in an amount of at least 10² CFU genetically modified Hs microbe per plant or plant part, between 10² and 10³ CFU per plant or plant part, at least 10³ CFU per plant or plant part, between 10³ and 10⁴ CFU per plant or plant part, at least 10⁴ CFU per plant or plant part, between 10⁴ and 10⁵ CFU per plant or plant part, at least 10⁵ CFU per plant or plant part, between 10⁵ and 10⁶ CFU per plant or plant part, between 10⁵ and 10⁹ CFU per plant or plant part, at least 10⁶ CFU per plant or plant part, between 10⁶ and 10⁷ CFU per plant or plant part, at least 10⁷ CFU per plant or plant part, between 10⁷ and 10⁸ CFU per plant or plant part, or greater than 10⁸ CFU per plant or plant part.

In particular embodiments, the formulation is applied to the plant part in an amount of at least 10² CFU genetically modified Hs microbe per seed, between 10² and 10³ CFU per seed, at least 10³ CFU per seed, between 10³ and 10⁴ CFU per seed, at least 10⁴ CFU per seed, between 10⁴ and 10⁵ CFU per seed, at least 10⁵ CFU per seed, between 10⁵ and 10⁶ CFU per seed, at least 10⁶ CFU per seed, between 10⁶ and 10⁷ CFU per seed, at least 10′ CFU per seed, between 10⁷ and 10⁸ CFU per seed, at least 10⁸ CFU per seed, between 10⁸ and 10⁹ CFU per seed, at least 10⁹ CFU per seed, between 10⁹ and 10¹⁰ CFU per see, at least 10¹⁰ CFU per seed, between 10¹⁰ and 10¹¹ CFU per seed, at least 10¹¹ CFU per seed, or greater than 10¹¹ CFU per seed.

Particular embodiments provide for a composition including a plant or plant part associated with a genetically modified Hs microbe of the disclosure. The composition can include a formulation of the genetically modified Hs microbe as described herein. In particular embodiments, the plant part includes a seed and the formulation includes a seed coating. Seed coatings can include: polymers, guar, film coating layers, binders, active ingredients (e.g., herbicides, plant growth regulators, crop dessicants, fungicides, bactericides, bacteriostats, insecticides, insect repellants, adjuvants, surfactants, fertilizers), filler, and nutrients. Seed coatings are described in, e.g., WO 2012/118795; WO 2010/111309; and U.S. Pat. No. 8,685,886. In particular embodiments, the composition can further include a medium that promotes plant growth. Media to promote plant growth typically allows ample drainage, permits air around plant roots, allows enough water for the plants, provides nutrients, and supports the plant. In particular embodiments, media that promotes plant growth include: soil, nitrogen, peat, peat-like material, bark, coconut coir, wood residues, bagasse, rice hulls, sand, perlite, pumice, vermiculite, calcined clays, hydrogel, expanded polystyrene, and urea formaldehydes. In particular embodiments, media that promotes plant growth include soil-less hydroponic growing media. In particular embodiments, hydroponic growing media include: rockwool, grow rock (lightweight expanded clay aggregate), coconut fiber, coconut chips, perlite, venniculite, oasis cubes, floral foam, growstone (e.g., recycled glass), river rock, pine shavings, composted and aged pine bark, polyurethane foam insulation, water-absorbing crystals, sand, and rice hulls.

Particular embodiments provide for a commodity plant product, as well as methods for producing a commodity plant product, that is derived from a plant of the present disclosure. A commodity plant product includes any composition or product that includes material derived from a plant, seed, plant cell, or other plant part of the present disclosure. Commodity plant products may be sold to consumers and can be viable or nonviable. Nonviable commodity products include: nonviable seeds and grains; processed seeds, seed parts, and plant parts; dehydrated plant tissue, frozen plant tissue, and processed plant tissue; seeds and plant parts processed for animal feed for terrestrial and/or aquatic animal consumption; oil, meal, flour, flakes, bran, fiber, paper, tea, coffee, silage, crushed or whole grain, and any other food for human or animal consumption; biomasses and fuel products; and raw material in industry. Industrial uses of oils derived from the agricultural plants described herein include ingredients for paints, plastics, fibers, detergents, cosmetics, lubricants, and biodiesel fuel. Commodity plant products also include industrial compounds, such as a wide variety of resins used in the formulation of adhesives, films, plastics, paints, coatings and foams.

### (iv) Methods of Use

The compositions and formulations including a genetically modified Hs microbe of the present disclosure may be used in a method to associate or contact the genetically modified Hs microbe with a plant or plant part. In particular embodiments, the genetically modified Hs microbe is associated or contacted with a plant or plant part by inoculation. Inoculation includes introducing a genetically modified Hs microbe onto or into a plant or plant part by any method of association. Methods of association or contacting can include: seed treatment, root wash, seedling soak, foliar application, soil inoculation, in-furrow application, side dress application, soil pre-treatment, wound inoculation, drip tape irrigation, vector-mediation via a pollinator, injection, osmopriming, hydroponics, aquaponics, and aeroponics. In particular embodiments, inoculation includes contacting a plant or plant part with a formulation or composition including a genetically modified Hs microbe described herein. Contacting or associating includes bringing a plant or plant part together with a formulation or composition including a genetically modified Hs microbe described herein such that the plant or plant part is touching or in physical contact with the formulation or composition to allow nitrogen fixed by the Hs microbe to be used by the plant or plant part to improve an agronomic trait of a plant grown from the inoculated plant or plant part.

Particular embodiments provide for a method for preparing a composition described herein, including associating or contacting the surface of a plant or plant part with a genetically modified Hs microbe of the present disclosure to produce a composition including an inoculated plant or plant part and a genetically modified Hs microbe of the present disclosure. In particular embodiments, the genetically modified Hs microbe is present in a formulation. In particular embodiments, the genetically modified Hs microbe is in an amount capable of improving an agronomic trait of the plant grown from the inoculated plant or plant part.

Any number of single carrier compositions and single methods of association or contacting, as well as combinations of carrier compositions and methods of association or contacting, are intended to be within the scope of the present disclosure. In some embodiments the Hs is associated with a seed using any method known in the art, for example, enrobing, co-drying, coating, infusing, injecting, and the like, prior to planting the seed in soil. In particular embodiments, application of the genetically modified Hs microbe to the plant may be achieved as a powder for surface deposition onto plant leaves, as a spray to the whole plant or selected plant part, as part of a drip to the soil or the roots, or as a coating onto a plant part prior to planting. In particular embodiments, a plant part may first become associated with a genetically modified Hs microbe by virtue of seed treatment with a solid (dry) formulation including a genetically modified Hs microbe, and upon germination and leaf emergence, the plant is then subjected to a foliar spray of a liquid formulation including a genetically modified Hs microbe. In particular embodiments, a plant may become associated with a genetically modified Hs microbe by virtue of inoculation of the growth medium (soil or hydroponic) with a liquid or solid formulation including a genetically modified Hs microbe, and be subjected to repeated (two, three, four, five, or more subsequent) inoculations with a liquid or solid formulation including a genetically modified Hs microbe. In particular embodiments, a plant seed is drenched with a formulation including a genetically modified Hs microbe.

In particular embodiments, the genetically modified Hs microbe disclosed herein can move from one plant tissue type to another, such as from the seed exterior into the vegetative tissues of a plant, from the seed interior into the vascular tissue, or from the seed coat into the leaf tissue. In particular embodiments, the genetically modified Hs microbe disclosed herein is coated onto the seed of a plant and, upon germination of the seed into a vegetative state, localizes to a different tissue of the plant. In particular embodiments, a different tissue of a plant includes: the root, adventitious root, seminal root, root hair, shoot, leaf, flower, bud, tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In particular embodiments, the genetically modified Hs microbe localizes to the root and/or the root hair of the plant. In particular embodiments, the genetically modified Hs microbe localizes to the photosynthetic tissues, for example, leaves and shoots of the plant. In particular embodiments, the genetically modified Hs microbe localizes to the vascular tissues of the plant, for example, in the xylem and phloem. In particular embodiments, the genetically modified Hs microbe localizes to the reproductive tissues (flower, pollen, pistil, ovaries, stamen, fruit) of the plant. In particular embodiments, the genetically modified Hs microbe localizes to the root, shoots, leaves and reproductive tissues of the plant. In particular embodiments, the genetically modified Hs microbe colonizes the plant such that it is present on the surface of the plant (i.e., its presence is detectably present on the plant exterior, or the episphere of the plant). In particular embodiments, the genetically modified Hs microbe localizes to substantially all, or all, tissues of the plant. In particular embodiments, the genetically modified Hs microbe does not localize to the root of a plant. In particular embodiments, the genetically modified Hs microbe does not localize to the photosynthetic tissues of the plant.

In particular embodiments, the genetically modified Hs microbe replicates within the host plant and colonizes the plant. In particular embodiments, the genetically modified Hs microbe colonizes a fruit or seed tissue of the plant. Successful colonization can be confirmed by detecting the presence of the bacterial population within the plant. For example, after applying the genetically modified Hs microbe to the seeds, high titers of the genetically modified Hs microbe can be detected in the roots and shoots of the plants that germinate from the seeds. Detecting the presence of the genetically modified Hs microbe inside the plant can be accomplished by measuring the viability of the genetically modified Hs microbe after surface sterilization of the seed or the plant: genetically modified Hs microbe colonization results in an internal localization of the genetically modified Hs microbe, rendering it resistant to conditions of surface sterilization. The presence and quantity of the genetically modified Hs microbe can also be established using other means known in the art, for example, immunofluorescence microscopy using microbe-specific antibodies, or fluorescence in situ hybridization (see, for example, Amann et al. (2001) Current Opinion in Biotechnology 12:231-236). Alternatively, specific nucleic acid probes recognizing conserved sequences from a genetically modified Hs microbe can be employed to amplify a region, for example by quantitative PCR, and correlated to CFUs (Colony Forming Units) by means of a standard curve. In particular embodiments, a CFU refers to a unit used to estimate the concentration of a microbe in a test sample. The number of visible colonies (CFUs) present on an agar plate can be multiplied by the dilution factor to obtain a CFU per volume.

In particular embodiments, the genetically modified Hs microbe is disposed, for example, on the surface of an agricultural plant part, in an amount effective to be detectable in the mature agricultural plant. In particular embodiments, the genetically modified Hs microbe is disposed in an amount effective to be detectable in an amount of at least 10.000 CFU, between 30,000 and 40,000 CFU, at least 50,000 CFU, between 50,000 and 60,000 CFU, at least 60,000 CFU, between 60,000 and 70,000 CFU, at least 70,000 CFU, between 70,000 and 80,000 CFU, at least 200,000 CFU, between 200,000 and 800,000 CFU, at least 1 M CFU, between 1 M and 1.2 M CFU, at least 10 M, between 10M and 10.5 M CFU, or more per gram of plant dry weight.

In particular embodiments, the genetically modified Hs microbe colonizes particular plant parts or tissue types of the plant. In particular embodiments, the genetically modified Hs microbe is disposed on the seed or seedling in an amount effective to be detectable within a target tissue of the mature agricultural plant selected from a fruit, a seed, a leaf, or a root, or portion thereof. For example, the genetically modified Hs microbe can be detected in an amount of between 30,000 and 40,000 CFU, at least 50.000 CFU, between 50,000 and 60,000 CFU, at least 60,000 CFU, between 60,000 and 70,000 CFU, at least 70,000 CFU, between 70,000 and 80,000 CFU, at least 200,000 CFU, between 200,000 and 800,000 CFU, at least 1 M CFU, between 1 M and 1.2 M CFU, at least 10 M, between 10M and 10.5 M CFU, in the target tissue of the mature agricultural plant on a per gram of dry weight of the target tissue.

The compositions and formulations of the present disclosure may be used to improve any characteristic of any agricultural plant. In particular embodiments, the present disclosure includes the use of a genetically modified Hs microbe disclosed herein to confer a beneficial agronomic trait upon a plant part or plant with which the genetically modified Hs microbe is associated.

In particular embodiments, the agronomic trait includes increased nitrogen fixation, reduced nitrogen usage, increased nitrogen content, increased plant yield, increased plant biomass, increased shoot biomass, increased shoot length, increased dry shoot weight, increased fresh shoot weight, increased seedling shoot length, increased dry seedling weight, increased fresh seedling weight, increased leaf surface area, increased root biomass, increased root length, increased root surface area, increased germination rate, increased emergence rate, increased photosynthetic capability, increased chlorophyll content, increased vigor, increased seed yield, increased dry weight of mature seeds, increased fresh weight of mature seeds, increased number of mature seeds per plant, increased number of pods per plant, increased length of pods per plant, increased plant height, increased pathogen resistance, increased pest resistance, earlier or increased flowering, increased protein content, increased carbohydrate content, and/or increased antioxidant content relative to a reference agricultural plant grown under the same conditions. In particular embodiments, the reference agricultural plant includes an uninoculated plant. In particular embodiments, at least two agronomic traits are improved in the agricultural plant. “Yield” or “plant yield” refers to increased plant growth, increased crop growth, increased biomass, and/or increased plant product production, and is dependent to some extent on temperature, plant size, organ size, planting density, light, water and nutrient availability, and how the plant copes with various stresses, such as through temperature acclimation and water or nutrient use efficiency. Examples of measurements of crop production include increased bushels per acre, increased corn kernels or soybeans per plant, or (kernels per ear)×(ears per acre)/(kernels per bushel)=(bushels/acre). In instances where the yield of a plant is used as a comparator one of ordinary skill in the art will appreciate that the dryness between the test tissue and the comparator needs to be comparable or accounted for.

In particular embodiments, the genetically modified Hs microbe may provide an improved benefit, e.g., increased plant yield, to a plant associated with the microbe that is of at least 3%, between 3% and 5%, between 3% and 20%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, or at least 300% or more, when compared with a reference agricultural plant (or population of plants) grown under the same conditions. In particular embodiments, the reference agricultural plant includes an uninoculated plant or population of uninoculated plants, or a plant or population of plants that is associated with the non-engineered parental microbe strain.

Particular embodiments provide plants, and fields of plants, that are associated with a genetically modified Hs microbe, such that the overall fitness, productivity or health of the plant or a portion thereof, is maintained, increased and/or improved over a period of time. Improvement in overall plant health can be assessed using numerous physiological parameters including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof. Improved plant health, or improved field health, can also be demonstrated through improved resistance or response to a given stress, either biotic or abiotic stress, or a combination of one or more abiotic stresses.

Particular embodiments provide a method of reducing nitrogen fertilizer application, including growing a plant from a plant part that has been contacted with a formulation including a genetically modified Hs microbe described herein, wherein application of nitrogen to the plant is reduced as compared to application of nitrogen to a reference agricultural plant. In particular embodiments, the reference agricultural plant is a corresponding plant grown from a plant part that has not been contacted with the formulation. A corresponding plant includes a plant of the same genus and/or species (e.g., a population of corn plants contacted with a genetically engineered Hs microbe described herein is compared to a population of corn plants that have not been contacted with a genetically engineered Hs microbe described herein). “Fertilizer” refers to any organic material or inorganic material of natural or synthetic origin which is added to soil to provide nutrients, including all three elements of nitrogen, phosphorus, and potassium, necessary to sustain plant growth. In particular embodiments, a fertilizer includes nitrogen.

In particular embodiments, the reduction in the application of nitrogen is measured as N replacement per application, and wherein the N replacement is at least 5 ppm of N, at least 6 ppm of N, at least 7 ppm of N, at least 8 ppm of N, at least 9 ppm of N, at least 10 ppm of N, at least 11 ppm of N, at least 12 ppm of N, at least 13 ppm of N, at least 14 ppm of N, at least 15 ppm of N, at least 16 ppm of N, at least 25 ppm of N, at least 50 ppm of N, at least 75 ppm of N, at least 100 ppm of N per application, or greater. In particular embodiments, the reference agricultural plant includes an uninoculated plant or population of uninoculated plants, or a plant or population of plants that is associated with the non-engineered parental microbe strain.

Particular embodiments provide a method of improving an agronomic trait in a plant, including growing a plant from a plant or plant part that has been contacted with a formulation including a genetically modified Hs microbe described herein, wherein an agronomic trait is improved in the plant as compared to the corresponding agronomic trait in a reference agricultural plant. In particular embodiments, the reference agricultural plant includes an uninoculated plant or population of uninoculated plants, or a plant or population of plants that is associated with the non-engineered parental microbe strain. In particular embodiments, the reference agricultural plant includes a plant or population of plants that is associated with a different genetically engineered microbe strain. In particular embodiments, the improved trait can be an increase in overall biomass of the plant or plant part, including its fruit or seed. In particular embodiments, a genetically modified Hs microbe is disposed on the surface or within a plant or plant part in an amount effective to increase the biomass of the plant or plant grown from the plant part. The increased biomass is useful in the production of commodity products derived from the plant. The increase in biomass can occur in a plant part (e.g., the root tissue, shoots, leaves, etc.), or can be an increase in overall biomass. In particular embodiments, an increase in overall biomass of a plant associated with a genetically modified Hs microbe can include an increase of at least 3%, between 3% and 5%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, as compared to overall biomass from a reference agricultural plant (or population of plants) grown under the same conditions. In particular embodiments, the reference agricultural plant includes an uninoculated plant or population of uninoculated plants, or a plant or population of plants that is associated with the non-engineered parental microbe strain. In particular embodiments, the reference agricultural plant includes a plant or population of plants that is associated with a different genetically engineered microbe strain. In particular embodiments, such increase in overall biomass can be under relatively stress-free conditions. In particular embodiments, the increase in biomass can be in plants grown under any number of abiotic or biotic stresses, including drought stress, salt stress, heat stress, cold stress, low nutrient stress, nematode stress, insect herbivory stress, fungal pathogen stress, bacterial pathogen stress, and viral pathogen stress.

In particular embodiments, a genetically modified Hs microbe disclosed herein is disposed in an amount effective to increase chlorophyll content (e.g., as measured by a SPAD meter). In particular embodiments, an increase in chlorophyll content of a plant associated with a genetically modified Hs microbe can include an increase of at least 3%, between 3% and 5%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, as compared to chlorophyll content from a reference agricultural plant (or population of plants) grown under the same conditions. In particular embodiments, the reference agricultural plant includes an uninoculated plant or population of uninoculated plants, or a plant or population of plants that is associated with the non-engineered parental microbe strain. In particular embodiments, the reference agricultural plant includes a plant or population of plants that is associated with a different genetically engineered microbe strain.

In particular embodiments, a genetically modified Hs microbe disclosed herein is disposed in an amount effective to increase shoot fresh weight. In particular embodiments, an increase in shoot fresh weight of a plant associated with a genetically modified Hs microbe can include an increase of at least 3%, between 3% and 5%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, as compared to shoot fresh weight from a reference agricultural plant (or population of plants) grown under the same conditions. In particular embodiments, the reference agricultural plant includes an uninoculated plant or population of uninoculated plants, or a plant or population of plants that is associated with the non-engineered parental microbe strain. In particular embodiments, the reference agricultural plant includes a plant or population of plants that is associated with a different genetically engineered microbe strain.

In particular embodiments, a genetically modified Hs microbe disclosed herein is disposed in an amount effective to increase shoot dry weight. In particular embodiments, an increase in shoot dry weight of a plant associated with a genetically modified Hs microbe can include an increase of at least 3%, between 3% and 5%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, as compared to shoot dry weight from a reference agricultural plant (or population of plants) grown under the same conditions. In particular embodiments, the reference agricultural plant includes an uninoculated plant or population of uninoculated plants, or a plant or population of plants that is associated with the non-engineered parental microbe strain. In particular embodiments, the reference agricultural plant includes a plant or population of plants that is associated with a different genetically engineered microbe strain.

In particular embodiments, a genetically modified Hs microbe disclosed herein is disposed in an amount effective to increase root biomass of a plant associated with the genetically modified Hs microbe by at least 3%, between 3% and 5%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, as compared to root biomass of a reference agricultural plant (or population of plants) grown under the same conditions. In particular embodiments, the reference agricultural plant includes an uninoculated plant or population of uninoculated plants, or a plant or population of plants that is associated with the non-engineered parental microbe strain. In particular embodiments, the reference agricultural plant includes a plant or population of plants that is associated with a different genetically engineered microbe strain.

In particular embodiments, a genetically modified Hs microbe is disposed on the surface or within a plant or plant part in an amount effective to increase the average biomass of the fruit or car from the resulting plant at least 3%, between 3% and 5%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, as compared to biomass of the fruit or ear of a reference agricultural plant (or population of plants) grown under the same conditions. In particular embodiments, the reference agricultural plant includes an uninoculated plant or population of uninoculated plants, or a plant or population of plants that is associated with the non-engineered parental microbe strain. In particular embodiments, the reference agricultural plant includes a plant or population of plants that is associated with a different genetically engineered microbe strain.

In particular embodiments, a genetically modified Hs microbe is disposed on the surface or within a plant or plant part in an amount effective to increase the photosynthetic capability of the resulting plant by at least 3%, between 3% and 5%, between 3% and 20%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, as compared to photosynthetic capability of a reference agricultural plant grown under the same conditions. In particular embodiments, photosynthetic capability can be expressed as a rate at which leaves are able to fix carbon during photosynthesis. Photosynthetic capability is typically measured as the amount of carbon dioxide that is fixed per meter squared per second, for example as μmol/m2/sec. Assays to measure photosynthetic capability include leaf gas exchange, thermal imagery, hyperspectral reflectance, chlorophyll fluorescence, normalized difference vegetation index (NDVI) and infrared thermography. In particular embodiments, photosynthetic capability can be indirectly measured by measuring plant yield, plant biomass, shoot biomass, shoot length, dry shoot weight, fresh shoot weight, seedling shoot length, dry seedling weight, fresh seedling weight, leaf surface area, number of stomata per leaf, root biomass, root length, root surface area, germination rate, emergence rate, chlorophyll content, vigor, seed yield, dry weight of mature seeds, fresh weight of mature seeds, number of mature seeds per plant, number of pods per plant, length of pods per plant, plant height, or a combination thereof.

Particular embodiments provide for genetically modified Hs microbe-associated plants with increased resistance to an abiotic stress. Exemplary abiotic stresses include: drought, heat, cold, salt stress, high metal content, and low nutrient.

Particular embodiments provide for genetically modified Hs microbe-associated plants with increased resistance to biotic stress. Exemplary biotic stresses include: insect infestation, nematode infestation, complex infection, fungal infection, bacterial infection, oomycete infection, protozoal infection, viral infection, and herbivore grazing, or a combination thereof.

Other plant traits can be improved when a plant or plant part is associated with a genetically modified Hs microbe disclosed herein. In particular embodiments, the genetically modified Hs microbe-associated plant can have an increase in plant growth hormones such as auxin as compared to a reference agricultural plant grown under the same conditions. In particular embodiments, the genetically modified Hs microbe-associated plant can have an altered hormone status or altered levels of hormone production as compared with a reference agricultural plant. An alteration in hormone status may affect many physiological parameters, including flowering time, water efficiency, apical dominance and/or lateral shoot branching, increase in root hair, and alteration in fruit ripening. In particular embodiments, the reference agricultural plant includes an uninoculated plant or population of uninoculated plants, or a plant or population of plants that is associated with the non-engineered parental microbe strain. In particular embodiments, the reference agricultural plant includes a plant or population of plants that is associated with a different genetically engineered microbe strain.

In particular embodiments, the genetically modified Hs microbe-associated plant can have an improved nutritional content of the plant or plant part as compared to a reference agricultural plant or plant part from a reference agricultural plant grown under the same conditions. Examples of such nutrients include: amino acid; protein; oil (including oleic acid, linoleic acid, alpha-linoleic acid, saturated fatty acids, palmitic acid, stearic acid and trans fats); carbohydrates (including sugars such as sucrose, glucose and fructose, starch, or dietary fiber); Vitamin A; thiamine (vitamin B1); riboflavin (vitamin B2); nitrogen content, niacin (vitamin B3); pantothenic acid (vitamin B5); vitamin B6; folate (vitamin B9); choline; vitamin C; vitamin E; vitamin K; calcium; iron; magnesium; manganese; phosphorus; potassium; sodium; and zinc.

In particular embodiments, the genetically modified Hs microbe-associated plant can have a reduced content of a harmful or undesirable substance as compared with a reference agricultural plant. Such compounds include those which are harmful when ingested in large quantities or are bitter tasting (for example, oxalic acid, amygdalin, certain alkaloids such as solanine, caffeine, nicotine, quinine and morphine, tannins, cyanide). As such, in particular embodiments, the genetically modified Hs microbe-associated plant or part thereof contains less of the undesirable substance as compared with a reference agricultural plant. In particular embodiments, the improved trait can include improved taste of the genetically modified Hs microbe-associated plant or part thereof, including the fruit or seed.

The association between a genetically modified Hs microbe disclosed herein and a plant can be detected using methods known in the art. For example, the biochemical, metabolomics, proteomic, genomic, epigenomic and/or transcriptomic profiles of a genetically modified Hs microbe-associated plant can be compared with the corresponding profile of a reference agricultural plant grown under the same conditions. In particular embodiments, the reference agricultural plant includes an uninoculated plant or population of uninoculated plants. In particular embodiments, the reference agricultural plant includes a plant or population of plants that is associated with the non-engineered parental microbe strain. Transcriptome analysis of a genetically modified Hs microbe disclosed herein and reference agricultural plants can also be performed to detect changes in expression of at least one transcript, or a set or network of genes upon microbe association. Similarly, epigenetic changes can be detected using methylated DNA immunoprecipitation followed by high-throughput sequencing.

Metabolomic differences between the plants can be detected using methods known in the art. Metabolites, proteins, or other compounds can be detected using any suitable method, including: gel electrophoresis; liquid chromatography; gas phase chromatography; mass spectrometry; nuclear magnetic resonance (NMR); immunoassays (e.g., enzyme-linked immunosorbent assays (ELISAs)); chemical assays; spectroscopy; optical imaging techniques (such as magnetic resonance spectroscopy (MRS); magnetic resonance imaging (MRI); CAT scans; ultrasound; and mass spectrometry-based tissue imaging or X-ray detection methods (e.g., energy dispersive x-ray fluorescence detection)). In particular embodiments, commercial systems for chromatography and NMR analysis are utilized. Such metabolomic methods can be used to detect differences in levels or content of, for example, hormones, nutrients, secondary metabolites, root exudates, phloem sap, xylem sap, and heavy metals. Such methods are also useful for detecting alterations of metabolites in the genetically modified Hs microbe.

The term “plant” is used in its broadest sense. It includes any species of grass (e.g., turf grass), sedge, rush, ornamental or decorative, crop or cereal, fodder or forage, fruit or vegetable, fruit plant or vegetable plant, flowers, and trees. In particular embodiments, a plant includes: wheat, soybean, maize, barley, millet, rice, turfgrass, cotton, canola, rapeseed, alfalfa, tomato, sugarbeet, oats, rye, sorghum, almond, walnut, apple, peanut, strawberry, lettuce, orange, potato, banana, sugarcane, cassava, mango, guava, palm, onions, olives, peppers, tea, yams, cacao, sunflower, asparagus, carrot, coconut, lemon, lime, watermelon, cabbage, cucumber, and grape. A plant part is any part of a plant, tissue of a plant, or cell of a plant. In particular embodiments, a plant or plant part includes: a whole plant, a seedling, cotyledon, meristematic tissue, ground tissue, vascular tissue, dermal tissue, seed, pod, tiller, sprig, leaf, stomata, root, shoot, stem, flower, fruit, pistil, ovaries, pollen, stamen, phloem, xylem, stolon, plug, bulb, tuber, corm, keikis, bud, and blade. “Leaf” and “leaves” refer to a usually flat, green structure of a plant where photosynthesis and transpiration take place and attached to a stem or branch. “Stem” refers to a main ascending axis of a plant. “Seed” refers to a ripened ovule, including the embryo and a casing. In particular embodiments, compositions of the present disclosure include a plant and/or a plant part described herein.

Particular embodiments provide for a plant to which a genetically modified Hs microbe-associated plant described herein is compared to assess agronomic trait improvement or reduction in nitrogen application for the genetically modified Hs microbe-associated plant. In particular embodiments, a reference agricultural plant includes a plant or population of plants of the same genus and species as the genetically modified Hs microbe-associated plant and grown under the same conditions but that has not been associated with a genetically modified Hs microbe (i.e., an uninoculated plant or population of uninoculated plants). In particular embodiments, a reference agricultural plant includes an uninoculated plant or population of uninoculated plants of the same genus and species as the genetically modified Hs microbe-associated plant and grown under the same conditions and that is a commercially grown plant. In particular embodiments, a commercially grown plant is a plant that is or has been grown by farmers and sold as a commodity. In particular embodiments, a reference agricultural plant includes a plant or population of plants of the same genus and species as the genetically modified Hs microbe-associated plant and grown under the same conditions and that is associated (i.e., inoculated) with a different genetically modified Hs microbe. In particular embodiments, a different genetically modified Hs microbe includes different genetic modifications in its genome as compared to the genetically modified Hs microbe associated with the test plant.

### (v) Exemplary Embodiments

1. A genetically modified Herbaspirillum seropedicae (Hs) microbe including:
(a) overexpression of transcriptional activator NifA; and/or
(b) downregulation of endogenous amtB gene encoding ammonium transporter AmtB.

2. The genetically modified Hs microbe of embodiment 1, wherein the downregulation of the endogenous amtB gene includes a deletion of the endogenous ammonium transporter amtB gene.

3. The genetically modified Hs microbe of embodiment 2, wherein the deletion of the endogenous ammonium transporter amtB gene includes replacement of the endogenous amtB gene with at least one heterologous nifA expression cassette such that expression of AmtB is reduced or eliminated.

4. The genetically modified Hs microbe of any of embodiments 1-3, wherein the overexpression of NifA includes the presence of at least one heterologous nifA gene.

5. The genetically modified Hs microbe of embodiment 4, wherein the at least one heterologous nifA gene is integrated at a neutral site in the Hs microbe genome.

6. The genetically modified Hs microbe of embodiment 5, wherein the neutral site includes HSERO_RS20140, HSERO_RS06930, HSERO_RS04675, HSERO_RS17925, HSERO_RS20445, HSERO_RS10460, HSERO_RS17440, or HSERO_RS24900.

7. The genetically modified Hs microbe of any of embodiments 4-6, wherein the heterologous nifA gene is operably linked to a heterologous constitutive promoter.

8. The genetically modified Hs microbe of embodiment 7, wherein the heterologous constitutive promoter is P65, CP25, or CP32.

9. The genetically modified Hs microbe of embodiment 7 or 8, wherein the heterologous constitutive promoter is P65.

10. The genetically modified Hs microbe of any of embodiments 1-9, wherein the overexpression of NifA includes endogenous nifA gene operably linked to a heterologous constitutive promoter, wherein the heterologous constitutive promoter replaces the endogenous nifA promoter.

11. The genetically modified Hs microbe of embodiment 10, wherein the heterologous constitutive promoter is P65, CP25, or CP32.

12. The genetically modified Hs microbe of embodiment 10 or 11, wherein the heterologous constitutive promoter is CP25.

13. The genetically modified Hs microbe of any of embodiments 1-12, further including downregulation of endogenous glnK gene encoding PII-like nitrogen regulatory protein.

14. The genetically modified Hs microbe of embodiment 13, wherein the downregulation of the endogenous glnK gene includes a deletion of the endogenous glnK gene.

15. The genetically modified Hs microbe of any of embodiments 1-14, further including downregulation of endogenous ntrC gene encoding nitrogen regulatory protein NtrC.

16. The genetically modified Hs microbe of embodiment 15, wherein the downregulation of the endogenous ntrC gene includes a deletion of the endogenous ntrC gene.

17. The genetically modified Hs microbe of any of embodiments 1-16, further including endogenous glnA gene encoding glutamine synthetase (GS) attenuated in expression as compared to an Hs microbe that is not genetically modified.

18. The genetically modified Hs microbe of embodiment 17, wherein the attenuated expression of the GS includes expression of the endogenous glnA gene operably linked to a heterologous promoter.

19. The genetically modified Hs microbe of embodiment 18, wherein the heterologous promoter is an inducible promoter.

20. The genetically modified Hs microbe of embodiment 19, wherein the inducible promoter includes a Ptac promoter.

21. The genetically modified Hs microbe of any of embodiments 17-20, wherein the attenuated expression of the GS includes expression of the endogenous glnA gene operably linked to a ribosomal binding site (RBS) modified to decrease translation efficiency of the endogenous glnA gene as compared to the endogenous glnA gene operably linked to the corresponding endogenous unmodified RBS.

22. The genetically modified Hs microbe of any of embodiments 17-21, wherein the attenuated expression of the GS includes expression of the endogenous glnA gene operably linked to a degradation tag.

23. The genetically modified Hs microbe of embodiment 22, wherein the degradation tag includes an ssRA/tmRNA tag.

24. The genetically modified Hs microbe of embodiment 23, wherein the ssRA/tmRNA tag includes the sequence as set forth in SEQ ID NO: 10.

25. The genetically modified Hs microbe of any of embodiments 1-24, further including an amino (N)-terminal truncation of the endogenous glnE gene encoding an adenylyl transferase.

26. The genetically modified Hs microbe of any of embodiments 1-25, wherein nitrogenase activity of the genetically modified Hs microbe is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to nitrogenase activity of a control Hs microbe as measured by an acetylene reduction assay, an ¹⁵N₂ fixing assay, an ¹⁵N dilution assay, and/or an ammonia biosensor assay.

27. The genetically modified Hs microbe of any of embodiments 1-26, wherein ammonia secretion from the genetically modified Hs microbe is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to ammonia secretion of a control Hs microbe as measured by an ammonia biosensor assay.

28. The genetically modified Hs microbe of embodiment 26 or 27, wherein the control Hs microbe is an Hs microbe that is not genetically modified.

29. The genetically modified Hs microbe of embodiment 27 or 28, wherein the ammonia biosensor assay includes an Hs microbe including a deletion of endogenous nifH gene and a detectable reporter expression cassette.

30. The genetically modified Hs microbe of any of embodiments 1-29, wherein the genetically modified Hs microbe does not include a selectable marker or a counter-selection marker.

31. A formulation including a genetically modified Hs microbe of any of embodiments 1-30 and a carrier.

32. The formulation of embodiment 31, wherein the formulation includes a stabilizer, a surfactant, an adherent, a fungicide, a nematicide, a bactericide, an insecticide, an herbicide, a virucide, a nutrient, or a combination thereof.

33. The formulation of embodiment 31 or 32, wherein the formulation is a seed coating.

34. A method of inoculating a plant or plant part, including contacting a plant or plant part with a formulation of any of embodiments 31-33.

35. The method of embodiment 34, further including growing the inoculated plant or plant part.

36. A plant or plant part produced by the method of embodiment 34 or 35.

37. A method of improving an agronomic trait in a plant, including growing a plant from a plant or plant part that has been contacted with a formulation of any of embodiments 31-33.

38. The method of embodiment 37, further including administering nitrogen fertilizer to the plant.

39. A method of reducing nitrogen fertilizer application, including inoculating a plant or plant part with a formulation of any of embodiments 31-33, and growing a plant from the inoculated plant or plant part, wherein application of nitrogen to the grown plant is reduced as compared to application of nitrogen to a reference agricultural plant.

40. The method of embodiment 39, wherein the reduction in the application of nitrogen (N) is measured as N replacement per application, and wherein the N replacement is at least 5 ppm of N, at least 6 ppm of N, at least 7 ppm of N, at least 8 ppm of N, at least 9 ppm of N, at least 10 ppm of N, at least 11 ppm of N, at least 12 ppm of N, at least 13 ppm of N, at least 14 ppm of N, at least 15 ppm of N, at least 16 ppm of N, or greater.

41. A method for preparing a composition, including contacting the surface of a plant or plant part with a formulation of any of embodiments 31-33 to produce an inoculated plant or plant part including the genetically modified Hs microbe, wherein the genetically modified Hs microbe is present in the formulation in an amount capable of improving an agronomic trait of the plant grown from the inoculated plant or plant part.

42. The method of embodiment 41, wherein the plant part includes a root, a stem, or a leaf.

43. A composition including a plant or plant part and a genetically modified Hs microbe of any of embodiments 1-30.

44. The composition of embodiment 43, wherein the plant part is a seed and the genetically modified Hs microbe is part of a seed coating.

45. The composition of embodiment 43 or 44, wherein the genetically modified Hs microbe is present in an amount of 10⁵-10⁹ CFU per plant or plant part.

46. The composition of any of embodiments 43-45, wherein the plant or plant part includes: a whole plant, a seedling, meristematic tissue, ground tissue, vascular tissue, dermal tissue, seed, leaf, root, shoot, stem, flower, fruit, stolon, bulb, tuber, corm, keikis, or bud.

47. The composition of any of embodiments 43-46, wherein the plant or plant part is from: maize, wheat, soybean, barley, millet, rice, turfgrass, cotton, canola, rapeseed, alfalfa, tomato, sugarbeet, oats, rye, sorghum, almond, walnut, apple, peanut, strawberry, lettuce, orange, potato, banana, sugarcane, potato, cassava, mango, guava, palm, onions, olives, peppers, tea, yams, cacao, sunflower, asparagus, carrot, coconut, lemon, lime, watermelon, cabbage, cucumber, and grape.

48. A composition of any of embodiments 43-47, further including a medium that promotes plant growth.

49. A plant grown from the composition of any of embodiments 43-48, wherein the plant exhibits an improved agronomic trait as compared to a reference agricultural plant, and wherein the improved agronomic trait includes: increased nitrogen fixation, reduced nitrogen usage, increased plant yield, increased plant biomass, increased shoot biomass, increased shoot length, increased dry shoot weight, increased fresh shoot weight, increased seedling shoot length, increased dry seedling weight, increased fresh seedling weight, increased leaf surface area, increased root biomass, increased root length, increased root surface area, increased germination rate, increased emergence rate, increased photosynthetic capability, increased chlorophyll content, increased vigor, increased seed yield, increased dry weight of mature seeds, increased fresh weight of mature seeds, increased number of mature seeds per plant, increased number of pods per plant, increased length of pods per plant, increased plant height, increased pathogen resistance, increased pest resistance, earlier or increased flowering, increased protein content, increased carbohydrate content, increased antioxidant content, or a combination thereof.

50. The plant of embodiment 49, wherein the plant has a 3 to 20% increase in dry shoot weight as compared to the plant not grown from the composition.

51. A seed coating including a genetically modified Hs microbe of any one of embodiments 1-30.

52. A genetic construct including 5′ to 3′:
(a) an upstream (US) homology arm including sequence homologous to sequence 5′ of the start codon of an endogenous Hs glnK coding sequence; and
(b) a downstream (DS) homology arm including sequence homologous to sequence 3′ of the stop codon of the endogenous Hs glnK coding sequence.

53. The genetic construct of embodiment 52, wherein the genetic construct includes a sequence having at least 90% sequence identity to SEQ ID NO: 1 or includes a sequence as set forth in SEQ ID NO: 1.

54. A genetic construct including 5′ to 3′:
(a) an upstream (US) homology arm including sequence homologous to sequence 5′ of the start codon of an endogenous Hs amtB coding sequence; and
(b) a downstream (DS) homology arm including sequence homologous to sequence 3′ of the stop codon of the endogenous Hs amtB coding sequence.

55. The genetic construct of embodiment 54, wherein the genetic construct includes a sequence having at least 90% sequence identity to SEQ ID NO: 4 or includes a sequence as set forth in SEQ ID NO: 4.

56. The genetic construct of embodiment 54 or 55, further including 5′ to 3′: (i) a constitutive promoter and (ii) an nifA coding sequence that is 3′ of the US homology arm and 5′ of the DS homology arm.

57. The genetic construct of embodiment 56, wherein the constitutive promoter is P65, CP25, or CP32.

58. The genetic construct of embodiment 56 or 57, wherein the constitutive promoter is P65.

59. The genetic construct of any of embodiments 56-58, wherein the genetic construct includes a sequence having at least 90% sequence identity to SEQ ID NO: 2 or includes a sequence as set forth in SEQ ID NO: 2.

60. The genetic construct of embodiment 56 or 57, wherein the constitutive promoter is CP32.

61. The genetic construct of any of embodiments 56, 57, or 60, wherein the genetic construct includes a sequence having at least 90% sequence identity to SEQ ID NO: 3 or includes a sequence as set forth in SEQ ID NO: 3.

62. A genetic construct including 5′ to 3′:
(a) an upstream (US) homology arm including sequence homologous to sequence 5′ of an endogenous Hs nifA promoter;
(b) a heterologous constitutive promoter; and
(c) a downstream (DS) homology arm including sequence homologous to sequence 3′ of the endogenous Hs nifA promoter.

63. The genetic construct of embodiment 62, wherein the constitutive promoter is P65, CP25, or CP32.

64. The genetic construct of embodiment 62 or 63, wherein the constitutive promoter is CP25.

65. The genetic construct of embodiment 62 or 64, wherein the genetic construct includes a sequence having at least 90% sequence identity to SEQ ID NO: 5 or includes a sequence as set forth in SEQ ID NO: 5.

66. A genetic construct including 5′ to 3′:
(a) an upstream (US) homology arm including sequence homologous to sequence 5′ of the start codon of an endogenous Hs glnA coding sequence;
(b) an inducible promoter; and
(c) a downstream (DS) homology arm including sequence homologous to sequence 3′ of the stop codon of the endogenous Hs glnA coding sequence.

67. The genetic construct of embodiment 66, wherein the inducible promoter includes a Ptac promoter.

68. The genetic construct of embodiment 66 or 67, further including lacI coding sequence.

69. The genetic construct of embodiment 68, wherein the lacI coding sequence is operably linked to a Plac promoter.

70. The genetic construct of any of embodiments 67-69, wherein transcription from the Ptac promoter is in the opposite direction from transcription of the lacI coding sequence.

71. The genetic construct of any of embodiments 66-70, wherein the genetic construct includes a sequence having at least 90% sequence identity to SEQ ID NO: 7 or includes a sequence as set forth in SEQ ID NO: 7.

72. A genetic construct including 5′ to 3′:
(a) an upstream (US) homology arm including sequence homologous to sequence 5′ of the start codon of an endogenous Hs nifH coding sequence; and
(b) a downstream (DS) homology arm including sequence homologous to sequence 3′ of the stop codon of the endogenous Hs nifH coding sequence.

73. The genetic construct of embodiment 72, wherein the genetic construct includes a sequence having at least 90% sequence identity to SEQ ID NO: 8 or includes a sequence as set forth in SEQ ID NO: 8.

74. A genetic construct including 5′ to 3′:
(a) an upstream (US) homology arm including sequence homologous to sequence 5′ of the start codon of an endogenous Hs nifA coding sequence; and
(b) a downstream (DS) homology arm including sequence 3′ of the stop codon of the endogenous Hs nifA coding sequence.

75. The genetic construct of embodiment 74, wherein the genetic construct includes a sequence having at least 90% sequence identity to SEQ ID NO: 9 or includes a sequence as set forth in SEQ ID NO: 9.

76. The genetic construct of any one of embodiments 52-75, wherein each homology arm is 2 kb in length.

77. The genetic construct of any one of embodiments 52-76, wherein the genetic construct further includes a selectable marker for selection in Hs.

78. The genetic construct of embodiment 77, wherein the selectable marker is a gentamicin resistance marker.

79. The genetic construct of any one of embodiments 52-78, wherein the genetic construct further includes a counter selection marker.

80. The genetic construct of embodiment 79, wherein the counter selection marker is human herpes simplex virus thymidine kinase.

81. A cell including the genetic construct of any of embodiments 52-80.

### (vi) EXAMPLES

### Example 1. Exemplary Gram-Negative Nitrogen Fixation Strains

This Example describes the Herbaspirillum seropedicae strains created to fix nitrogen in N replete or N deficient conditions.

Herbaspirillum seropedicae Z78 (Hs) is a gram-negative diazotrophic endophyte, originally isolated from sorghum. The nitrogen fixation pathway in this organism is shown in FIG. 1. Due to the high energy cost of nitrogen (N) fixation, cells normally repress these genes in the presence of bioavailable N (referred to here as “N replete” condition). A final commercial microbial product can be co-applied with fertilizer at planting, so exogenous N in the form of N-fertilizer can be present, requiring deregulation of the nitrogen fixation pathway genes by genetic modification of an Hs microbe to increase nitrogen fixation in the Hs microbe.

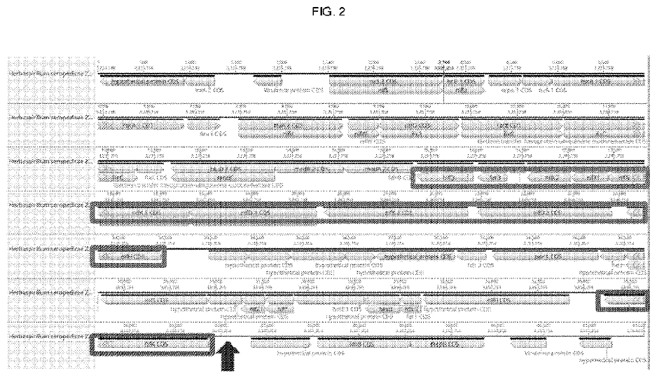

Nitrogenase and its required accessory proteins are encoded in a 37 kb nif cluster in Hs (FIG. 2), including multiple operons. The expression of some of these operons have been shown to be dependent on the transcriptional activator, NifA (Chubatsu et al. Plant and Soil 2012; 356(1-2):197-207). Although the complete nitrogen regulation of the nif cluster in Hs is unclear, the transcriptional activator protein NifA may be tightly regulated by the signal transduction PII nitrogen regulatory proteins GlnB/GlnK, and the Nitrogen regulatory protein C (NtrC) that binds to the NifA promoter (Chubatsu et al. Plant and Soil 2012; 356(1-2):197-207). Deleting GlnK and replacing the native promoter of nifA, PnifA, with a heterologous constitutive promoter can contribute to the removal of the NtrC mediated regulation of nifA, allowing its expression even in the presence of N.

A heterologous nifA gene under a constitutive promoter to drive expression even in the presence of NH₃ was inserted at the amtB locus, thereby deleting the amtB gene (“knock-in/knock-out”). This modification was expected to have two positive effects. First, by placing nifA under control of a nitrogen insensitive promoter, the pathway should continue to be expressed even under N replete conditions. Second, the targeted amtB gene encodes an ammonia transporter known to transport ammonia into the cell. Knock-out mutants of AmtB in Hs have been reported to exhibit nitrogenase activity even in the presence of ammonium, suggesting a potential role of this transporter in exogenous NH₃ sensing (Noindorf et al. Archives of Microbiology 2006; 185(1):55-62). Therefore, an amtB deletion could further relieve nitrogen repression on the nitrogenase pathway by preventing assimilation of exogenous ammonia (secreted or environmental) and reducing the cell's ability to detect ammonia in the environment.

Ammonia can be released extracellularly or be incorporated into amino acids via glutamine synthetase, GlnA, which converts ammonia and glutamate into glutamine. Reducing the activity of GlnA can reduce the incorporation of ammonia into glutamine, thereby increasing the available ammonia to plants. Therefore, an engineered Hs strain was created that includes the native glnA gene under the control of an inducible promoter, Ptac, by replacing the native glnA promoter with Ptac and expressing a LacI lactose regulator protein (using the Plac promoter). In the absence of an inducer compound such as IPTG (isopropyl-3-D-thiogalactopyranoside), expression of glnA is repressed by Lac binding to the Ptac promoter. Upon addition of an inducer such as IPTG, the LacI regulator is bound by IPTG and its repressor function abrogated, allowing the expression of glnA from the Ptac promoter. This system allows testing of ammonia secretion by modulating glnA expression.

Genetic constructs. All genetic constructs, including knockouts and overexpression constructs, were made from the same suicide vector backbone, as shown in FIGS. 3A-3I. This vector was obtained via DNA synthesis and contains the following components: promoter driving beta lactamase (bla) gene from pUC19 vector (Pamp); ampicillin resistant marker for selection in E. coli from pUC19 vector (AmpR, bla gene); pBR322 on (from pUC19) or p15A on (from pACYC) origin of replication for plasmid replication in E. coli; promoter driving aacC1 gene encoding gentamicin selection marker (PaacC); gentamicin selection marker encoded by gentamicin 3-N-acetyltransferase (aacC1) from Pseudomonas aeruginosa for selection in Hs (GentR); promoter driving hsvTK gene (PJ23101; World Wide Web at parts.igem.org/Part:BBa_J23101); hsvTK gene for counter-selection in Hs (thymidine kinase human alpha herpesvirus 1; GenBank: ADM22345.1); and RP4 oriT origin of transfer for conjugation into Hs (from plasmid RP4 (IncPα); Pansegrau et al. J Mol Biol 1994; 239(5):623-663).

Overexpression constructs. For overexpression constructs such as ΔamtB::P65:nifA:TrrnB, the homology arms to amtB (ammonia transporter gene), the nifA coding sequence, P65 promoter and rrnB terminator were added to the suicide vector backbone (FIG. 3B). Each part was amplified by PCR either from genomic DNA or synthesized DNA and assembled into the final construct via circular polymerase extension cloning (Quan and Tian PLoS ONE 2009; 4(7):e6441). The homology arms to amtB were designed such that a 2 kb sequence upstream of the coding sequence of amtB (amtB US) and a 2 kb sequence downstream of amtB (amtB DS) were used to delete the coding sequence of amtB upon insertion of the overexpression construct (FIG. 4).

Deletion constructs. For deletion constructs such as ΔglnK, the homology arms to glnK were added to the suicide vector backbone. Similar to that for amtB deletion, a 2 kb sequence upstream of the coding sequence of glnK and a 2 kb sequence downstream of glnK were used to delete the coding sequence of glnK. The upstream and downstream sequences were amplified from a genomic DNA prep of Hs and assembled into the final construct via circular polymerase extension cloning.

ΔglnK. 2 kb upstream (US) and downstream (DS) homology arms of glnK coding sequence (CDS) was amplified from an Hs genomic DNA sample and assembled into the vector backbone (FIG. 3A; SEQ ID NO: 1 includes the glnK US homology arm—glnK DS homology arm sequence cassette and SEQ ID NO: 11 includes the glnK US homology arm—glnK DS homology arm sequence cassette in a vector including gentamicin selectable marker and human alpha herpesvirus 1 thymidine kinase counter-selectable marker). The ΔglnK construct was transformed into wt Hs strain (t403320) to obtain a ΔglnK strain.

ΔamtB::P65:nifA (Hs5). 2 kb US and DS homology arms of amtB were amplified from an Hs genomic DNA sample, P65 promoter was amplified from synthesized DNA, nifA was amplified from a genomic DNA sample, and all were assembled into the vector backbone (FIG. 3B includes the amtB US homology arm—P65 promoter—NifA CDS—amtB DS homology arm sequence cassette, which cassette was included in a vector including gentamicin selectable marker and human alpha herpesvirus 1 thymidine kinase counter-selectable marker). The ΔamtB::P65:nifA construct was transformed into wt Hs strain (t403320) to overexpress nifA.

ΔglnK ΔamtB::P65:nifA (Hs6). The ΔglnK construct was first transformed into wt Hs strain (t403320) to obtain a ΔglnK strain, and then the ΔglnK strain was transformed with a ΔamtB::P65:nifA construct to overexpress nifA.

ΔamtB::PCP32:nifA (Hs2). 2 kb US and DS homology arms of amtB were amplified from an Hs genomic DNA sample, CP32 promoter was amplified from synthesized DNA, nifA was amplified from a genomic DNA sample, and all were assembled into the vector backbone (FIG. 3C includes the amtB US homology arm—CP32 promoter—NifA CDS—amtB DS homology arm sequence cassette, which cassette was included in a vector including gentamicin selectable marker and human alpha herpesvirus 1 thymidine kinase counter-selectable marker). The ΔamtB::PCP32:nifA construct was transformed into wt Hs strain (t403320) to overexpress nifA.

ΔglnK ΔamtB::PCP32:nifA (Hs3). The ΔglnK construct was first transformed into wt Hs strain (t403320) to obtain a ΔglnK strain, and then the ΔglnK strain was transformed with a ΔamtB::PCP32:nifA construct to overexpress nifA.

ΔamtB. US and DS homology arms of amtB were amplified from an Hs genomic DNA sample and assembled into the vector backbone (FIG. 3D; SEQ ID NO: 4 includes the amtB US homology arm—amtB DS homology arm sequence cassette and SEQ ID NO: 14 includes the amtB US homology arm—amtB DS homology arm sequence cassette in a vector including gentamicin selectable marker and human alpha herpesvirus 1 thymidine kinase counter-selectable marker). The ΔamtB construct was transformed into wt Hs strain (t403320) to obtain a ΔamtB strain.

PnifA::PCP25. US and DS homology arms of the promoter region of nifA and CP25 promoter were synthesized and assembled into the vector backbone (FIG. 3E; SEQ ID NO: 5 includes the Hs NifA promoter US homology arm—CP25 promoter—Hs NifA promoter DS homology arm sequence cassette and SEQ ID NO: 15 includes the Hs NifA promoter US homology arm—CP25 promoter—Hs NifA promoter DS homology arm sequence cassette in a vector including gentamicin selectable marker and human alpha herpesvirus 1 thymidine kinase counter-selectable marker).

ΔamtB PnifA::PCP25. The ΔamtB construct was first transformed into wt Hs strain (t403320) to obtain an ΔamtB strain, and then the ΔamtB strain was transformed with a PnifA::CP25 construct to replace the native nifA promoter with the CP25 promoter.

ΔglnK PnifA::PCP25 ΔamtB::P65:nifA (Hs12). The ΔglnK construct was first transformed into wt Hs strain (t403320) to obtain a ΔglnK strain, and then the ΔglnK strain was transformed with a PnifA::CP25 construct to replace the native nifA promoter with CP25 promoter, and then the ΔglnK PnifA::PCP25 strain was transformed with the ΔamtB::P65:nifA construct to overexpress nifA.

ΔntrC. 2 kb US and DS homology arms of ntrC CDS were amplified from an Hs genomic DNA sample and assembled into the vector backbone (FIG. 3F; SEQ ID NO: 6 includes the ntrC US homology arm—ntrC DS homology arm sequence cassette and SEQ ID NO: 16 includes the ntrC US homology arm—ntrC DS homology arm sequence cassette in a vector including gentamicin selectable marker and human alpha herpesvirus 1 thymidine kinase counter-selectable marker). The ΔntrC construct was transformed into wt Hs strain (t403320) to obtain a ΔntrC strain.

ΔntrC ΔamtB::PCP32:nifA (Hs7). The ΔntrC construct was first transformed into wt Hs strain (t403320) to obtain an ΔntrC strain, and then the ΔntrC strain was transformed with a ΔamtB::PCP32:nifA construct to overexpress nifA.

ΔntrC ΔamtB::PCP32:nifA PnifA::PCP25 (Hs4). The ΔntrC construct was first transformed into wt Hs strain (t403320) to obtain an ΔntrC strain, and then the ΔntrC strain was transformed with a PnifA::CP25 construct to replace the native nifA promoter with CP25 promoter, and then the ΔntrC PnifA::PCP25 strain was transformed with the ΔamtB::PCP32:nifA construct to overexpress nifA.

ΔglnK PnifA::PCP25 ΔamtB::PCP32:nifA (Hs8). The ΔglnK construct was first transformed into wt Hs strain (t403320) to obtain a ΔglnK strain, and then the ΔglnK strain was transformed with a PnifA::CP25 construct to replace the native nifA promoter with CP25 promoter, and then the ΔglnK PnifA::PCP25 was transformed with the ΔamtB::PCP32:nifA construct to overexpress nifA.

ΔntrC ΔamtB::P65:nifA (Hs9). The ΔntrC construct was first transformed into wt Hs strain (t403320) to obtain an ΔntrC strain, and then the ΔntrC strain was transformed with the ΔamtB::P65:nifA construct to overexpress nifA. Hs10 is a sister clone to Hs9.

ΔntrC ΔamtB. The ΔntrC construct was first transformed into wt Hs strain (t403320) to obtain an ΔntrC strain, and then the ΔntrC strain was transformed with the ΔamtB construct to delete amtB.

Plac:lacI:Ptac:glnA. The Lac repressor coding sequence and Ptac promoter were synthesized, with 2 kb homology arms to the US and DS sequences of glnA promoter, and assembled into the vector backbone (FIG. 3G; SEQ ID NO: 7 includes the glnA promoter US homology arm—lac promoter—lacI CDS—tac promoter—glnA promoter DS homology arm sequence cassette and SEQ ID NO: 17 includes the glnA promoter US homology arm—lac promoter—lacI CDS—tac promoter—glnA promoter DS homology arm sequence cassette in a vector including gentamicin selectable marker and human alpha herpesvirus 1 thymidine kinase counter-selectable marker). The lac promoter-lacI CDS is arranged in the cassette such that the lacI is transcribed in the opposite direction from transcription from the Ptac promoter as shown in FIG. 3G.

ΔglnK ΔamtB::PCP32:nifA Plac:lacI:Ptac:glnA. The ΔglnK construct was first transformed into wt Hs strain (t403320) to obtain a ΔglnK strain, and then the ΔglnK strain was transformed with the ΔamtB::PCP32:nifA construct to overexpress nifA, and then the ΔglnK ΔamtB::PCP32:nifA strain was transformed with Plac:lacI:Ptac:glnA to operably link the native glnA gene under the control of an inducible LacI/Ptac promoter.

ΔntrC PnifA::PCP25 ΔamtB::P65:nifA (Hs11). The ΔntrC construct was first transformed into wt Hs strain (t403320) to obtain an ΔntrC strain, and then the ΔntrC strain was transformed with a PnifA::CP25 construct to replace the native nifA promoter with CP25 promoter, and then the ΔntrC PnifA::PCP25 was transformed with the ΔamtB::P65:nifA construct to overexpress nifA.

ΔnifH. 2 kb US and DS homology anns of the nifH CDS was amplified from an Hs genomic DNA sample and assembled into the vector backbone (FIG. 3H; SEQ ID NO: 8 includes the nifH US homology arm—nifH DS homology arm sequence cassette and SEQ ID NO: 18 includes the nifH US homology arm—nifH DS homology arm sequence cassette in a vector including gentamicin selectable marker and human alpha herpesvirus 1 thymidine kinase counter-selectable marker). The ΔnifH construct was transformed into wt Hs strain (t403320) to obtain a ΔnifH strain.

ΔnifA. 2 kb US and DS homology arms of the nifA CDS was amplified from an Hs genomic DNA sample and assembled into the vector backbone (FIG. 3I; SEQ ID NO: 9 includes the nifA US homology arm—nifA DS homology arm sequence cassette and SEQ ID NO: 19 includes the nifA US homology arm—nifA DS homology arm sequence cassette in a vector including gentamicin selectable marker and human alpha herpesvirus 1 thymidine kinase counter-selectable marker). The ΔnifA construct was transformed into wt Hs strain (t403320) to obtain a ΔnifA strain.

To build the engineered Hs strains, the DNA constructs were transformed into Hs via conjugation with an E. coli BW29427 donor strain (genotype described at World Wide Web at cgsc.biology.yale.edu/Strain.php?ID=143445). The BW29427 donor strain is auxotrophic for diaminopimelic acid.

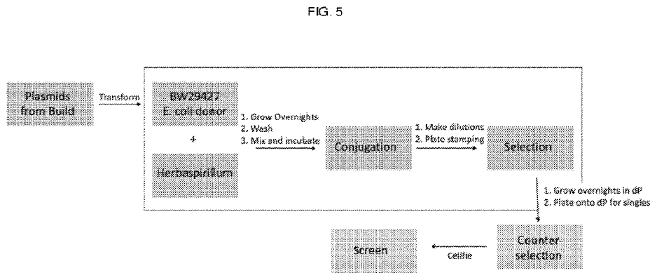

Protocol for Conjugation of Hs. The overall conjugation process of Hs for genomic integrations is shown in FIG. 5. The exemplary protocol includes (1) chemical transformation of a DNA construct into E. coli BW29427 donor strain and (2) conjugation of Hs with E. coli BW29427.

Transformation of DNA into E. coli BW29427. Any standard E. coli transformation protocol known in the art would work. The following describes an exemplary protocol. On day 1, an overnight culture of E. coli BW29427 was prepared. Briefly, a single colony of E. coli BW29427 was inoculated in 3 mL of LB+0.3 mM diaminopimelic acid (DAP)+100 μg/mL streptomycin media in a 12 mL culture tube. The culture tube was incubated at 37° C. with shaking at 180-250 RPM and was saturated overnight. On day 2, a 100 mL culture of E. coli BW29427 was prepared from the overnight culture. One mL of overnight culture was added to 100 mL of LB+0.3 mM DAP+streptomycin (100 μg/mL) media in a 500 mL flask. The flask was incubated at 37° C. with shaking at 180-250 RPM. The OD of the culture was monitored, and the culture was harvested at OD 0.4 (typically reached in 3 hours). The flask was incubated on ice for 10 min. then poured into two 50 mL falcon tubes and centrifuged at 3500×g for 10 min (4° C. preferred, room temperature acceptable). The supernatant was discarded, and the cell pellets were resuspended in 20 mL of cold 0.1M calcium chloride (CaCl₂). The centrifugation was repeated, the supernatant was discarded, and the cell pellets were resuspended in 10 mL of cold 0.1M CaCl₂. The cell suspension was allowed to sit on ice for 30 min and then centrifuged at 3500×g for 10 min. The supernatant was discarded, and the cell pellets were resuspended in 2.5 mL of cold 0.1M CaCl₂)+15% glycerol. The cell suspensions from both falcon tubes were combined to make a total of 5 mL. Fifty μL of cell suspension was aliquoted into each well of a 96-well plate on ice. The 5 mL cell suspension would be enough for one 96-well plate. Five μL of a DNA construct (i.e., plasmid, at concentration >2 ng/μL) was added to cells in each well and mixed by pipetting. The cell mixture was allowed to sit on ice for 10 min, and then the cells were heat shocked on a thermocycler at 42° C. for 30 sec. The cells were returned to ice for 5 min. Cells in each well were recovered with 150 μL of LB and transferred to a 96-deep well plate. The plate was incubated at 37° C. with shaking at 1000 RPM for 1 hour. Ten μL of cells from each well were plated on LB+0.3 mM DAP+100 μg/mL streptomycin+100 μg/mL carbenicillin agar plates. Cells were not diluted prior to plating.

Following the above E. coli transformation protocol, 3-10 colonies were obtained per 8 μL spot. The plate can be saved 4° C. for two weeks. A glycerol stock can be made from an overnight culture. An overnight culture is obtained by picking a colony into 1 mL of LB+0.3 mM DAP+100 μg/mL streptomycin+100 μg/mL carbenicillin and incubating at 37° C. overnight to obtain a dense culture. 250 μL of 50% glycerol is added to 250 μL of the dense culture to make the glycerol stock. Glycerol stocks are frozen at −80° C.

Conjugation of Hs with E. coli BW29427 containing integration constructs. On day 1, overnight cultures of Hs and E. coli were prepared. The overnight culture of Hs was prepared by picking a single colony of Hs from a plate and inoculating into 1 mL Nutrient Broth without antibiotics in a 96-deep well plate (Assay Block 2 mL deep well, Costar 07200700). The plate was incubated at 30° C. with shaking at 1000 RPM. An overnight culture of E. coli BW29427 containing plasmid for transformation was prepared by: picking a single colony of E. coli BW29427 from a plate and inoculating into 1 mL of LB+0.3 mM DAP+100 μg/mL streptomycin+100 μg/mL carbenicillin in a 96-deep well plate; and incubating the plate at 37° C. with shaking at 1000 RPM. Both cultures were saturated overnight. A typical OD 600 nm for an overnight culture of Hs is 0.8, while a typical OD 600 nm for an overnight culture of E. coli is 2.

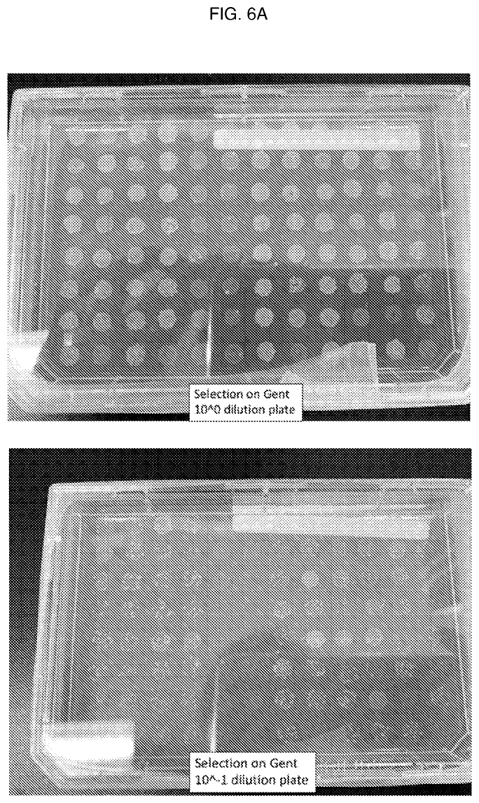

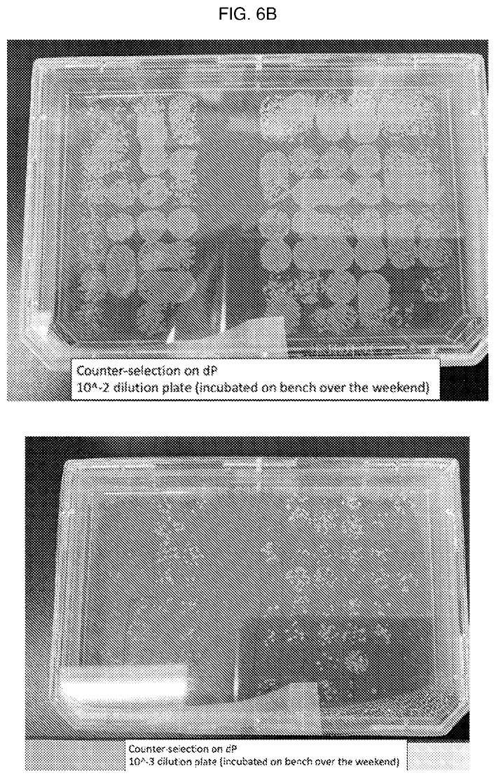

On day 2, both Hs and E. coli cells were centrifuged at 3500×g for 5 min and washed with sterile water twice, using 1 mL sterile water per well. It is important to ensure that all antibiotics from the E. coli culture are washed away before proceeding to the next step. Hs cells were resuspended in 25 μL of Nutrient Broth, and E. coli cells were resuspended in 50 μL of Nutrient Broth. Twenty-five μL of Hs cells were mixed with 25 μL of E. coli cells in a PCR 96-well plate. Control wells of only Hs cells and of only E. coli cells were prepared. The plate was covered with a breathable Aera-seal and incubated at 30° C. for 4 hours. Shaking is not necessary for this step. A 10× dilution series of the cell mixture, from 10⁰ to 10-2 were made in sterile water. Eight μL cells were spotted onto YPD+gentamycin 500 agar plates (SBS format) and incubated at 30° C. Colonies appeared in 2 days, and distinct single colonies can typically be picked from the 10⁻¹ to 10⁻² dilution plates. Typically, 10 distinct colonies/8 μL spot are observed on a 10⁻¹ dilution plate and 0-3 colonies/8 μL spot are observed on a 10⁻² dilution plate. No colonies were observed with Hs only and E. coli only controls. Pictures of plates from representative experiments are shown in FIG. 6A. With selection on gentamycin, the entire plasmid is integrated into the amtB genomic location via homologous recombination.

Since the E. coli donor strain is auxotrophic for diaminopimelic acid, plating on gentamicin plates without addition of diaminopimelic acid counter-selects against the donor strain. Similarly, only Hs cells carrying the gentamicin resistance gene via integration of the plasmid can survive. To remove the plasmid backbone and selection markers, transformants on the gentamicin plate were counter-selected for the presence of the hsvTK gene by culturing with 6-(b-D-2-deoxyribofuranosyl)-3,4-dihydro8H-pyrimido[4,5-c][1,2]oxazin-7-one (dP) nucleoside. The nucleoside analogue dP can be used to select against cells expressing a herpes simplex virus thymidine kinase (HSV-TK) since the enzyme incorporates dP into DNA, which effectively destroys the genetic information of the cells. This counter-selection step induces a second homologous recombination event that loops out the backbone and markers on the plasmid, leaving a clean insertion. The following steps in the protocol involves counter-selection on the non-natural nucleoside dP (Lin and Brown (1989) Nucleic Acids Res. 17(24):10373-10383) to induce the second crossover to complete the genomic integration. On day 4, Nutrient Broth+50 μM dP solution was prepared using a stock of 100 mM dP. One colony from the gentamycin plates described above was picked and inoculated into 1 mL of Nutrient Broth+50 μM dP in a 96-deep well plate. The plate was incubated at 30° C. with shaking at 1000 RPM. Cultures will be saturated overnight. On day 5, a dilution series from 10⁻¹ to 10⁻³ of cell cultures in the plate were made in sterile water. Eight L cells were spotted onto YPD+25 μM dP agar plates (SBS format) to obtain single colonies. It is important to obtain single colonies in this step because upon second crossover, some population of cells would revert to wild type (wt) while some population would obtain the clean deletion/insertion. Picking single colonies is essential to avoid a mixed population of wt and mutants. The agar plate was incubated at 30° C. Colonies appeared in 1-2 days. Typically, 1-20 distinct colonies/8 μL spot were observed on a 10-2 dilution plate and 0-4 colonies/8 μL spot were observed on a 10-3 dilution plate. Pictures of plates from representative experiments are shown in FIG. 6B. After colonies have appeared, the plate can be saved at 4° C. for two weeks. On day 7, 5-8 single colonies were picked and inoculated into 1 mL of Nutrient Broth in a 96-deep well plate. Each plate was stamped on YPD to save colonies for banking later, pending confirmation of integration. Site specific insertion in the recipient Hs genome was confirmed by sequencing.

Depending on the site of integration and/or the genomic modification, a percentage of the colonies will include the genomic modification. A glycerol stock for each successful integrant from the stamped plate was made by growing an overnight culture in Nutrient Broth and adding glycerol to a final concentration of 25%. The glycerol stock was stored at −80° C.

Genetic constructs used to create genetically engineered Hs microbes of the present disclosure are listed in Table 1 and components found in the genetic constructs are listed in Table 2.

TABLE 1
List of Hs Strain IDs, Genotype, and Purpose of Genetic Modification

| | | Purpose of Genetic |
|---|---|---|
| Strain ID | Genotype | Modification |
| Hsl | ΔnifH | control |
| Hs2 | ΔamtB::PCP32:nifA | express NifA even under N |
| | | replete conditions; prevent |
| | | assimilation of exogenous |
| | | ammonia (secreted or |
| | | environmental) and reduce the |
| | | cell's ability to detect ammonia |
| | | in the environment |
| Hs3 | ΔglnK ΔamtB::PCP32:nifA | removal of the NtrC mediated |
| | | regulation of nifA; express NifA |
| | | even under N replete conditions; |
| | | prevent assimilation of |
| | | exogenous ammonia (secreted or |
| | | environmental) and reduce the |
| | | cell's ability to detect ammonia |
| | | in the environment |
| Hs4 | ΔntrC ΔamtB:PCP32:nifA PnifA:PCP25 | removal of the NtrC mediated |
| | | regulation of nifA; express NifA |
| | | even under N replete conditions; |
| | | prevent assimilation of |
| | | exogenous ammonia (secreted or |
| | | environmental) and reduce the |
| | | cell's ability to detect ammonia |
| | | in the environment |
| Hs5 | ΔamtB::P65:nifA | express NifA even under N |
| | | replete conditions; prevent |
| | | assimilation of exogenous |
| | | ammonia (secreted or |
| | | environmental) and reduce the |
| | | cell's ability to detect ammonia |
| | | in the environment |
| Hs6 | ΔglnK ΔamtB::P65:nifA | removal of the NtrC mediated |
| | | regulation of nifA; express NifA |
| | | even under N replete conditions; |
| | | prevent assimilation of |
| | | exogenous ammonia (secreted or |
| | | environmental) and reduce the |
| | | cell's ability to detect ammonia |
| | | in the environment |
| Hs7 | ΔntrC ΔamtB::PCP32:nifA | removal of the NtrC mediated |
| | | regulation of nifA; express NifA |
| | | even under N replete conditions; |
| | | prevent assimilation of |
| | | exogenous ammonia (secreted or |
| | | environmental) and reduce the |
| | | cell's ability to detect ammonia |
| | | in the environment |
| Hs8 | ΔglnK PnifA::PCP25 ΔamtB::PCP32:nifA | removal of the NtrC mediated |
| | | regulation of nifA; express NifA |
| | | even under N replete conditions; |
| | | prevent assimilation of |
| | | exogenous ammonia (secreted or |
| | | environmental) and reduce the |
| | | cell's ability to detect ammonia |
| | | in the environment |
| Hs9 and | ΔntrC ΔamtB::P65:nifA | removal of the NtrC mediated |
| Hs10 (sister | | regulation of nifA; express NifA |
| clones) | | even under N replete conditions; |
| | | prevent assimilation of |
| | | exogenous ammonia (secreted or |
| | | environmental) and reduce the |
| | | cell's ability to detect ammonia |
| | | in the environment |
| Hsl1 | ΔntrC PnifA::PCP25 ΔamtB::P65:nifA | removal of the NtrC mediated |
| | | regulation of nifA; express NifA |
| | | even under N replete conditions; |
| | | prevent assimilation of |
| | | exogenous ammonia (secreted or |
| | | environmental) and reduce the |
| | | cell's ability to detect ammonia |
| | | in the environment |
| Hs12 | ΔglnK PnifA::PCP25 ΔamtB:P65:nifA | removal of the NtrC mediated |
| | | regulation of nifA; express NifA |
| | | cven under N replete conditions; |
| | | prevent assimilation of |
| | | exogenous ammonia (secreted or |
| | | environmental) and reduce the |
| | | cell's ability to detect ammonia |
| | | in the environment |
| Intermediate | ΔamtB PnifA::PCP25 | express NifA even under N |
| | | replete conditions; prevent |
| | | assimilation of exogenous |
| | | ammonia (secreted or |
| | | environmental) and reduce the |
| | | cell's ability to detect ammonia |
| | | in the environment |
| Intermediate | PnifA::CP25 | express NifA even under N |
| | | replete conditions |
| Intermediate | ΔntrC ΔamtB | removal of the NtrC mediated |
| | | regulation of nifA; prevent |
| | | assimilation of exogenous |
| | | ammonia (secreted or |
| | | environmental) and reduce the |
| | | cell's ability to detect ammonia |
| | | in the environment |
| Intermediate | ΔglnK ΔamtB::PCP32:nifA Plac:lacI: Ptac:glnA | removal of the NtrC mediated |
| | | regulation of nifA; express NifA |
| | | even under N replete conditions; |
| | | prevent assimilation of |
| | | exogenous ammonia (secreted or |
| | | environmental) and reduce the |
| | | cell's ability to detect ammonia |
| | | in the environment; express |
| | | native GlnA with an inducible |
| | | LacI/Ptac promoter |
| Intermediate | ΔntrC | removal of the NtrC mediated |
| | | regulation of nifA |
| Control | ΔnifA | Negative control |

TABLE 2
Components of genetic constructs and source of component

| | | Source | |
|---|---|---|---|
| Name | Description | Organism | Reference for Component |
| CP25 | Constitutive | Borrelia | Stevenson et al. J Bacteriol |
| | promoter | burgdorferi | 1997; 179(13): 4285-4291 |
| glnA | Glutamine | Herbaspirillum | GenBank AM490508.1 |
| | synthetase | seropedicae Z78 | |
| LacI | Lactose operon | E. coli | GenBank MN443779.1; .1 |
| | repressor | | QGV56732 |
| P65 | Constitutive | Eggerthella lenta | Johns et al. Nature Methods |
| | promoter | VPI 0255, DSM | 2018; 15:323329 |
| | | 2243 | E. lenta complete genome at |
| | | | GenBank CP001726.1 |
| CP32 | Constitutive | Borrelia | Stevenson et al. J Bacteriol |
| | promoter | burgdorferi | 1997; 179(13): 4285-4291 |
| PnifA | Promoter of nifA | Herbaspirillum | From complete genome |
| | | seropedicae Z78 | sequence of Hs Z67 at |
| | | | GenBank |
| | | | CP011930.1 |
| Plac | Promoter of | E. coli | Czarniecki et al. J Bacteriol |
| | lactose operon | | 1997; 179(2): 423-429 |
| Ptac | Hybrid promoter | E. coli | de Boer et al. Proc. Natl. |
| | derived from trp | | Acad. Sci USA 1983; |
| | and lac | | 80: 21-25 |
| | promoters, | | |
| | also recognized | | |
| | by LacI | | |

### Example 2. Nitrogenase Activity and Nitrogen Fixation of Engineered Hs Strains as Measured by Assays

The engineered Hs strains were assessed by two different in vitro assays to measure the effects of the genetic modifications on different aspects of the nitrogen fixation pathway. In all cases, experiments were conducted with the wt strain and a strain with NifA deleted (ΔnifA) that served as a negative control.

Ammonia biosensor assay. This assay uses an Hs “indicator” strain that constitutively expresses a green fluorescent protein (GFP) and is modified to be NH₃ auxotrophic. The ammonia produced by the candidate strain being evaluated will diffuse through the membrane into the broth and will be assimilated by the Hs indicator strain. As the indicator strain grows, the GFP fluorescence will increase proportionally. The fluorescence measurement of this “indicator strain” allows measurement of extracellular ammonia concentration when mixed with a strain of interest. A detailed protocol of an ammonia biosensor assay is described below.

With reference to FIG. 7, an Hs indicator strain, ΔnifH::pConst-dsGreen, does not grow in the absence of ammonium and exhibits a low signal from green fluorescent protein (GFP). In the presence of ammonium, the indicator strain grows and exhibits a high signal from GFP. Thus, in an indicator (ammonia biosensor) assay, an experimental strain can be tested for its ability to secrete ammonia by mixing it with the indicator strain. If an experimental strain does not secrete ammonia (strain Hs-1 in FIG. 7), the indicator strain will not grow and low GFP signal is detected. If an experimental strain does secrete ammonia (strain Hs-2 in FIG. 7), the indicator strain will grow and high GFP signal is detected. The assays use control and experimental strains. Control strains include: a high biological positive; wt; a ΔnifA negative control; and the indicator strain. In particular embodiments, a high biological positive gives a fluorescence signal that is 40× above that of the wild type strain.

On day 1, cultures were prepared from glycerol stocks as follows. Glycerol stocks were removed from the freezer. In a biosafety cabinet (BSC), a sterile loop was used to remove a loopful of each control and streaked onto a YPD plate. 8-10 μL of each experimental strain was streaked or stamped onto YPD plates. The streaked or stamped plates were labeled and stored upside down in a 30° C. incubator for 2 days until colonies formed. Once sufficient colonies appeared, the plates were covered with parafilm and stored at 4° C. for up to one week.

On day 3, overnight cultures were prepared from colonies of the control and experimental strains as follows. Plates with 500 μL/well NfbHP+N media were prepared. Using a 1000 μL pipette tip, one colony was picked per strain from YPD plates (except for the indicator strain) and placed into each media well. The controls were inoculated into row 12, leaving at least two wells empty for indicator only and media only controls. The plate was covered and allowed to incubate 2 days at 30° C., 4% oxygen. In a BSC, a single colony of indicator strain was removed from YPD with a 5 μL sterile loop and placed into a flask with at least 30 mL NfbHP+N media. The flask was labeled and allowed to incubate 2 days at 30° C., 4% oxygen.

On day 5, an indicator experiment was set up from overnight cultures as follows. Plates were washed 2× with sterile distilled water and centrifuged at 3500×g for 5 min as follows. 500 μL sterile de-ionized (DI) H₂O was added to the wells and mixed to resuspend biomass.

The plates were covered and placed on a plate shaker (1000 rpm) for five min to fully resuspend the cells. The plates were centrifuged 3500×g for 5 min. The replenishing of DI H₂O, the shaking, and the centrifugation was repeated. Each strain was concentrated by resuspending in 100 μL sterile DI H₂O.

The plates were covered and placed on a plate shaker (1000 rpm) for five min to fully resuspend the cells.

The indicator strain was washed 2× with sterile distilled water as follows. In a BSC, the 30 mL culture was split into three 15 mL Eppendorf falcon tubes, with 10 mL per tube. The tubes were centrifuged for 10 min at 3500×g, the supernatant was discarded, and each cell pellet was resuspended with 5 mL sterile distilled water. The centrifugation and discarding of supernatant were repeated. Each cell pellet was then resuspended in 2 mL sterile distilled water and combined into one falcon tube (for a total of 6 mL). A 20× dilution plate was made to read OD600 as follows. 190 L/well sterile distilled water and 10 L/well prepared concentrated culture were aliquoted into a clear 96-well plate. In any remaining empty wells on the plate, 190 μL/well sterile distilled water and 10 μL condensed indicator stock were aliquoted.

The OD600 reading was taken, and a normalization factor was calculated as follows. A Synergy™ H1 microplate reader computer program with JF_OD_600 protocol was opened (BioTek, Highland Park, Winooski, VT). The plate was placed in the Synergy™ H1 microplate reader and the OD600 was read. The plate map of the OD600 data was copied and pasted into Excel. The average OD600 was calculated for experimental wells, control wells, and indicator wells. All readings were multiplied by 20× to calculate OD600 of undiluted samples. The amount of prepared cell stock needed to create a final OD600 reading of 0.1 for a final volume of 500 μL was calculated using the formula: (OD)i*(v)i=(OD)f*(v)f (i=initial, f=final, v=volume). For example, if the OD600 reading of a 20× diluted cell sample was 0.18, then the concentrated stock average OD600 (initial) is 3.6. For a final OD600 of 0.1 in a final volume of 500 μL, the initial volume of prepared bacterial stock per well would be 14 μL (3.6*(v)i=(0.1) (500)). The amount of indicator needed to create a final OD600 reading of 0.001 for a final volume of 500 μL was calculated using the same formula. For example, if the initial OD600 is 2.44, the final OD600 is 0.001, and the final volume is 500 μL, then the initial volume of indicator sample would be 0.2 μL (2.44*(v)i=(0.001)(500)). 0.2 μL is too small to pipette accurately. To address this, the indicator sample was diluted 100× (dilute 100 μL indicator in 9.9 mL water). In this case, 20 μL/well was used when setting up the experimental plate.

An indicator assay was set up as follows. To a new 96-deep well plate, the following was added: 500 μL N-free (nitrogen-free) NfbHP was added per well; an amount of prepared bacterial stock calculated as described above; an amount of prepared 100× dilution indicator stock calculated as described above; at least one media only well (blanks); and one indicator+media only well. The 96-deep well plate was labeled, covered, and incubated at 30° C., 4% O₂, 165 rpm.

On day 11, a read assay after 6 days of growth was conducted as follows. Wells were mixed with a pipette and 50 μL per well were aliquoted into black-walled, clear bottom 96-well plates. Fluorescence was measured using a Biotek Synergy H1 Microplate reader at an excitation/emission wavelength of 485/520 nm.

¹⁵N dilution assay. The assay measures incorporation of fixed nitrogen into amino acids. Cells were initially grown on rich media containing a ¹⁵N enriched nitrogen source. As the cells grew in this media, they incorporated ¹⁵N in the biomass. After reaching a target biomass, cells were transferred to a nitrogen free medium (under regular atmosphere). As the cells grew, they now incorporated atmospheric ¹⁴N into their biomass, thus diluting the initial pool of ¹⁵N. Arginine was used as a marker and the ratio of ¹⁴N-Arg/¹⁵N-Arg was obtained. A detailed protocol of an ¹⁵N dilution assay is described below.

Overnight cultures of Hs were grown from a colony into NfbHP+20 mM (¹⁵NH₄)₂SO₄ media overnight at 30° C., 4% oxygen, 125 RPM shaking. The next day, cultures were passed into fresh NfbHP+20 mM (¹⁵NH₄)₂SO₄ media, by adding 10 μL of overnight culture into 1000 μL of fresh media. This process was repeated three times to ensure that cells were fully labeled with ¹⁵N. After three passages, cultures were spun down at 3500×g for 5 minutes and the supernatant was discarded. Cell pellets were frozen until ready for liquid chromatograph-mass spectrometer (LCMS) analysis.

Briefly, cell pellets were hydrolyzed at 104° C. overnight in 6M hydrochloric acid. Once dried, sodium bicarbonate is added to re-suspend the hydrolyzed biomass and the supernatant was run on the LCMS with a Thermo Accucore HILIC 100 column, using water+0.1% formic acid and acetonitrile+0.1% formic acid as solvents. The amount of ¹⁵N labeled arginine species are then quantified and the percent of ¹⁴N arginine species is used to determine the nitrogen fixation ability of strains.

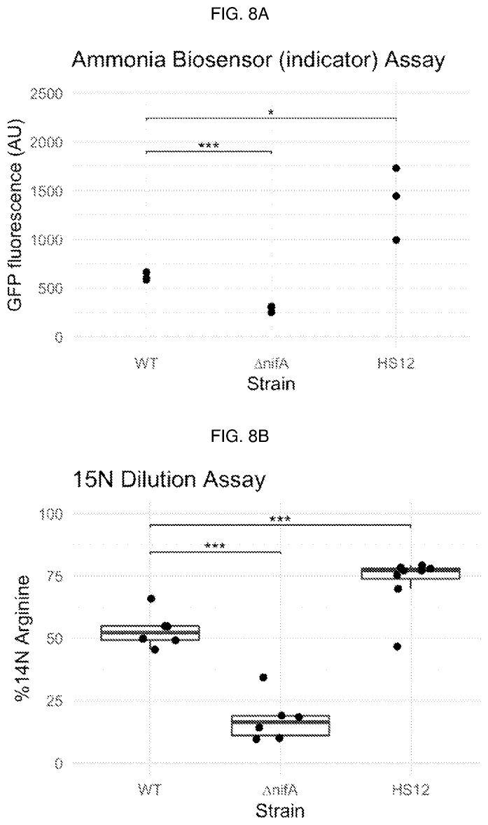

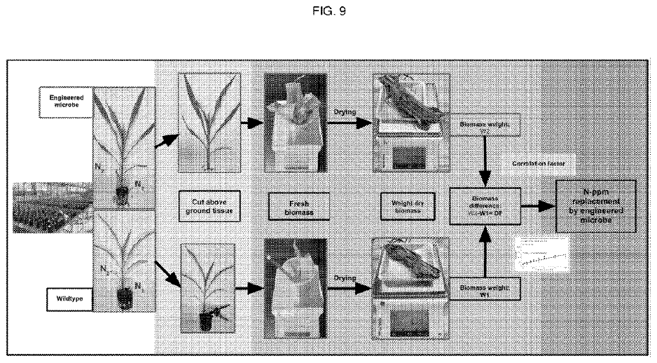

Results. FIGS. 8A-8B shows the results of Hs12 in the 2 assays, in comparison to wt and Hs ΔnifA negative control. In the two assays, Hs12 showed improvement over wt. Results are summarized in Table 3, where values reported are mean values.

TABLE 3
Summary Table of Hs12 Performance in in vitro Assays
and Fold Improvement Over wt.

| | | | | | | ¹⁵N | | |
|---|---|---|---|---|---|---|---|---|
| | | | | | | dilution | | |
| | | | NH3 | P-value | Fold | assay | | Fold |
| | P-value | Fold | biosensor | (vs | improvement | (% ¹⁴N | P-value | improvement vs |
| Strain | (vs wt) | improvement vs wt | (unit) | wt) | vs wt | Arg) | (vs wt) | wt |
| WT | — | 1 | 619 | — | 1 | 53.4 | — | 1 |
| Hs12 | 0.45 | 1.2 | 1390 | 0.07 | 2.2 | 72.7 | 0.003 | 1.4 |

The ammonia biosensor assay showed a 2-fold improvement fluorescence of the engineered Hs12 over wt (p-value=0.07) (FIG. 8A), indicating that more ammonia is secreted extracellularly. This could be primarily attributed to the deletion of the AmtB ammonia transporter, but also to the extra copy of nifA.

In the ¹⁵N dilution assay (FIG. 8B), Hs12 showed a 1.4-fold increase in %¹⁴N-Arg incorporation compared to wt (p-value=0.003), suggesting an increased nitrogen fixation capability of this engineered strain.

These results collectively show that the deregulation of the expression of the N-fixation genes in Hs12 leads to an increased nitrogen fixation ability, and consequently to an improved extracellular ammonia secretion in vitro. Based on these observations, this strain was further tested in planta.

### Example 3. In Planta Performance in the Greenhouse

In planta phenotype determination experimental design and statistical data analysis. A high level in planta experimental plan is shown below in Table 4 and FIG. 9. The greenhouse (GH) experiments were designed to measure effect of the genetically modified Hs microbes on plant phenotype.

TABLE 4
High Level Specifications of Greenhouse Experiments for In Planta Phenotype
Determination.

| Number of Independent Experiments Per | 3 Independent Experiments Per Strain on |
|---|---|
| Strain | Corn |
| Number of replicates per experiment | 16 corn plants |
| Soil type, volume per pot | Sungro, 800 mL/pot |
| Inoculation | Germinated corn seed drench |
| | 10⁹ cfu/seed |
| Initial Nitrogen application (sensitivity | 25 ppm (allowing detection of 4-5 ppm |
| estimate) | differences) |
| Greenhouse conditions | Photoperiod: 15 h |
| | Lighting: >225 w/m²* |
| | Humidity *: day: 50-55%; night: 70-75% |
| | Temperature: heating SP 60F, cooling SP 80F |
| | Soil moisture: 60% |
| | *may vary based on seasonal conditions |
| Phenotype determination | Engineered versus wild type of corn shoot dry |
| | weight difference. |
| | Extrapolated N-replacement. |
| Controls | Mock: corn inoculated with buffer only (no |
| | bacteria). |
| | Wild type (wt): corn inoculated with wild type |
| | bacteria. |
| Experiment statistical analysis | Randomized complete block design (RCBD), |
| | block as random factor, strain as fixed factor, |
| | linear mixed model, analysis of variance |
| | (ANOVA), Fisher's Least Significant Difference |
| | (LSD) test is used to compare engineered strains |
| | and wild type. |
| Inter-experimental statistical analysis method | Combining multiple independent RCBD |
| | experiments, adding experiment ID as another |
| | random factor to run the mixed model, and LSD |
| | test is used to compare strains and wild type |
| | differences across experiments. |
| Power simulation | Pilot experimental data were collected and used to |
| | simulate power (probability to detect treatment |
| | difference) at given variation level, number of |
| | replicates, and expected effect size. |

A power analysis was conducted to estimate the number of replicates and was set at 16 (further described below). Given 16 replicates, the screening experiment had 80% probability to find 6-10% plant weight difference (corresponding to 4 N-ppm supplemented) between engineered and wt strains.

Plant inoculation. Corn seeds were germinated for two days on moist filter paper prior to inoculation, to the point that the radicle emerged 1-3 cm. Uniform seedlings were selected for inoculation. Microbe strains of interest were streaked out on solid media from cryogenic stocks three days before plant inoculation, and grown overnight at 30° C. Two days before plant inoculation, a lawn of cells was scraped with a sterile loop and inoculated into 5 mL of media in a 14 mL culture tube and grown overnight. Hs strains were grown in Tryptic Soy Broth. One day before plant inoculation, 100 μL to 5 mL of the starter culture was added to 50-100 mL of appropriate media in a 250 mL baffled flask and grown overnight in a shaker at 250 rpm to reaching OD₆₀₀=1 by the following morning. On days of inoculation, cultures were centrifuged at 4,000×g for 10 min, media was decanted, and pellets were resuspended in PBS buffer. PBS alone was used as a negative control in all subsequent steps and was referred to as “mock”. Cultures were normalized to OD₆₀₀=1 (10⁹ CFU/mL), and two 200 μL samples were taken for serial dilution in PBS and plating on respective solid media to determine CFU/mL of inoculum.

The germinated seedlings were incubated with the corresponding inoculum treatments for 30 min at room temperature with mild shaking. Inoculated seedlings were planted into a 3-cm deep hole in the center of corresponding pots for each microbial treatment in the greenhouse. Then 1 mL of corresponding inoculum was drenched around each seedling. Extra pots were prepared for each microbial treatment in order to replace the pot(s) with abnormal seedling 4 days after planting. An automated irrigation system was used to deliver nutrient solutions with 25 ppm N at 60% soil moisture setting. Every pot and plant was closely monitored every day and abnormal events were recorded. In some earlier experiments, 0 ppm N and 100 ppm N were used.

Nutrient solution preparation. The nutrient stock solutions contained the following chemicals (per liter, they were 100× solution) of each nutrient delivered in the specified form as follows.

25 ppm Nitrogen nutrient solution stock solutions (100×)—Solution A: 12.38 g KH₂PO₄, 1.10 g NH₄H₂PO₄, 23.44 g MgSO₄, 5.15 g KNO₃, 41.74 g K₂SO₄. Solution B: 10.70 g Ca(NO₃)₂, 4.71 g CaCl₂), 1.51 g FeEDTA, 0.38 g MnEDTA, 0.24 g Na₂B₈O₁₃ 4H₂O per liter solution.

0 ppm Nitrogen nutrient solution stock solutions: (100×)—Solution A: 13.64 g KH₂PO₄, 23.44 g MgSO₄, 45.63 g K₂SO₄. Solution B: 6.71 g CaCl₂, 1.51 g FeEDTA, 0.38 g MnEDTA, 0.24 g Na₂B₈O₁₃ 4H₂O per liter solution.

100 ppm Nitrogen nutrient solution stock solution (100×)—Solution A: 11.63 g NH₄H₂PO₄, 23.44 g MgSO₄, 5.15 g KNO₃, 49.78 g K₂SO₄. Solution B: 50.13 g Ca(NO₃)₂, 1.51 g FeEDTA, 0.38 g MnEDTA, 0.24 g Na₂B₈O₁₃ 4H₂O per liter solution.

Before fertigation each time, the final solution volume was calculated based on the solution volume (mL) needed for each pot and the number of pots to be fertigated. Then 1 part of A solution and 1 part of B solution were mixed to 100 parts of the final solution.

Data collection. Growth stage (GWTPBTG): Leaf number was recorded weekly for each plant by counting fully expanded leaves. Plant height (PLTHT_tip): Plant height was measured weekly from soil surface to the tallest extended leaf tip (manually straightened up). Leaf greenness (CHLSPAD): One day before harvest, leaf greenness was measured on the upmost fully expanded leaf by using a SPAD meter. Four readings were taken in the middle portion of the leaf and the average of the 4 readings for each plant was recorded.

Shoot fresh weight (SHOOFWT): On day 28 after planting, all plants were watered early in the morning to make sure the soil in every pot was not dried to have uniform plant water content across the experiment. Plants were watered again early afternoon if the harvest was not completed in the morning. A plant was cut from the soil surface and shoot fresh weight measured immediately and placed into a paper bag with a plant tag on the bag for drying. All plants were cut uniformly from soil surface across the entire experiment.

Shoot dry weight (SHOODWT): Bags with plants were placed in the oven in the growth chamber room with 105° C. temperature for 15 min to stop all biological activities. Then the plant samples were dried with oven temperature at 75° C. until constant weight (weigh 10 bags from different positions in the oven with less than 1% weight decrease over 24 hours). Shoot dry weight (0% moisture) was taken immediately (within 30 seconds after the bag is removed from oven).

Greenhouse experiments were combined. For example, experiments can be 1-factor (strains only) or 2-factor (strains+N rates) RBCD with multiple experiments. The N rate was 25 ppm or was a combination of a number of N rates (e.g., 0, 25, 100 ppm N).

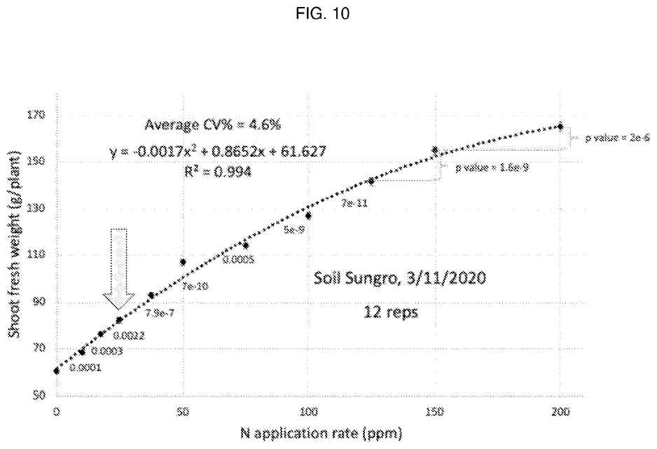

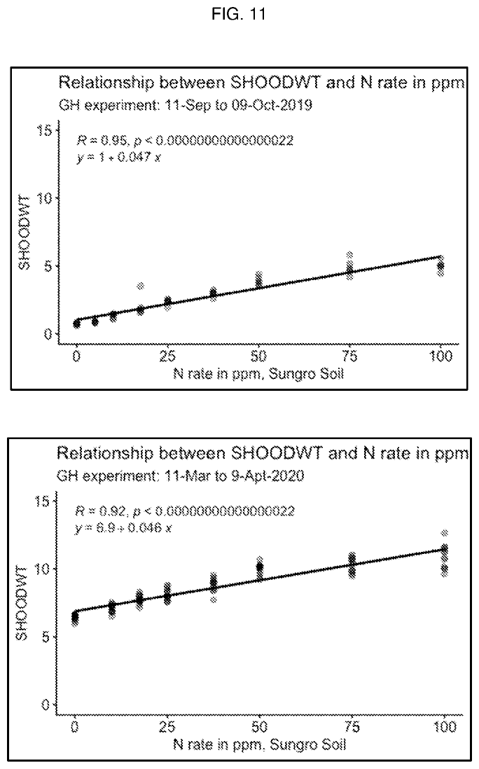

In planta greenhouse assay N-response. The response of corn fresh weight to various concentrations of nitrogen measured in parts per million (ppm) was evaluated. A concentration of supplemented N was selected that allowed for detection of microbial N supplementation, which was calculated from the observed dry biomass difference between plants treated with wild type and engineered strains. In particular, an N-dose was selected where stark differences in physiological parameters between nitrogen doses would be observed. Moreover, the experiment allowed determination of the sensitivity of N-response at the lower N concentrations. This work showed that a linear relationship between applied N and plant mass was observed within the range of 0 to 100 ppm of added N and that nitrogen dependent responses at the lower end of the dose response could be detected (FIG. 10, Sungro). This experiment was repeated twice in two different soil conditions (Turface, data not shown and Sungro) and a consistent slope was calculated for both datasets (FIG. 11; 0.047 and 0.046 gram increase per 1 ppm increase in N, Sungro shown), despite the difference in absolute means of biomass between the two experiments, leading to a different intercept in each regression line. Since plant growth and biomass accumulation responses to the fixed N microbiologically are the same as fertilizer N application, the value of these slopes allows for estimation of the approximate increase in N that would be required to achieve an equivalent increase in biomass. Based on the data, a correlation of 22 ppm N per 1 gram increase of plant biomass was observed. The 25 ppm N allowed for 1) the plants to exhibit clear nitrogen deprivation and 2) even modest nitrogen supplementation by the strain could be detected. Results showed that plants fertilized with 25 ppm N showed distinct nitrogen limitation phenotypes, and the assay allowed for reliable detection of 4-5 ppm differences. Therefore, 25 ppm N was selected as the basic N-condition for screening so as not to skew selection of strains based on physiological stress of the plants and to detect significant strain effects even if the nitrogen fixed by the strain was modest.

In planta power analysis. A power analysis was run to assess the ability to detect differences in the mean biomass in grams between engineered strains and wt. The analysis showed a power analysis for plants of large and smaller sizes. An initial power analysis was done using variance and mean fresh weight from model results of an experiment that produced the larger plants under treatment with the wt strain (M=107 g/plant, varE=63.8). Experimental protocols were expected to have plant biomass most similar to these larger plants. To detect a 10% increase in biomass (10.7 g) in plants of this size with 80% power at a significance level of 0.05, 8 replicates would be needed per level of N. For experiments that produce smaller plants (M=36.44 g/plant), a second power analysis was run using a smaller variance as seen in the results of earlier experiments (varE=13.26). Under these conditions, a 5 g increase in biomass would be detectable with 80% power at a significance level of 0.05, given the same number of replicates (8). An increase of 5 grams in fresh weight is equivalent to an increase of 4.6 ppm of applied N by a dose-response curve using fresh weight in grams as outcome.

These power calculations were done using least-squared means and residual variance as estimated by mixed model from a series of independent experiments. Mixed models for each experiment included applied N and strain as fixed effects, and replicate or block as random effect with fresh weight in grams as outcome. Fresh weight was used as the indicator for biomass in place of dry weight, as dry weight was not collected for some early experiments.

The above power analysis described the number of replicates that were determined for future experiments. Given 16 replicates, the screening experiment had 80% probability to find 6-10% plant fresh weight difference between engineered strains and wt. Some experiments included a higher number of replicates than recommended. This was due in part to an unexpected surplus of greenhouse resources which allowed for an increase in replicates that was greater than the experimental plan originally designed.

Results. The Hs12 strain was tested 3 times independently (16 replicates each time) and found to have a consistently positive in planta phenotype. Since variation occurs between experiments (seasonal change, culture conditions), an inter-experimental statistical analysis was used to bolster confidence in the results.

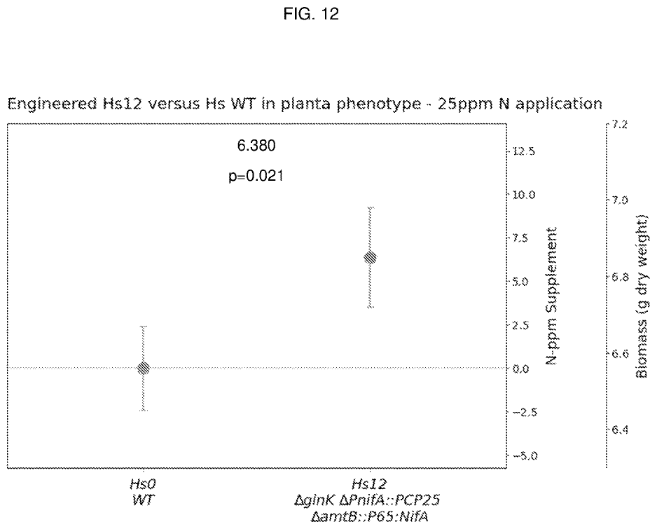

The effect of Hs12 on plants that received 25 ppm N-application rate in the standard GH fertigation was tested. The results shown in FIG. 12 are the result of an inter-experiment analysis run on 3 independent GH experiments. While consistent positive response for dry biomass gain over wt were observed, some of the independent experiments were not statistically significant (p-value>0.2). The overall dry weight of control plants varied across experiments. Combining multiple experiments (inter-experimental analysis) accounted for experimental variation due to microbial and seasonal difference and also allowed increased power to detect the mean difference between plants treated with wt and engineered strains. As a result, this inter-experiment analysis detected a statistically significant increase of dry biomass in plants treated with engineered strains when compared to those treated with wt microbes.

The results are summarized in Table 5 and shown in FIG. 12.

TABLE 5
Summary of Hs12 25 ppm N-application GH Experiment.

| | | Biomass | Biomass | N-ppm | Std | |
|---|---|---|---|---|---|---|
| | | (g dry | difference | supple- | Err | p- |
| Treatment | Genotype | weight) | (g) | mented | (g) | value |
| Hs0 | WT | 6.56 | — | — | — | — |
| Hs12 | ΔglnK, | 6.85 | 0.29 | 6.38 | 0.13 | 0.021 |
| | PnifA::PCP25, | | | | | |
| | amtB::P65:nifA | | | | | |

The evaluation of Hs2 across three independent experiments at a fixed N-application rate of 25 ppm showed a consistent improvement of biomass above the wt strain (FIG. 12).b The average N-ppm supplemented by the Hs12 strain across the 3 independent experiments was estimated to be 6.38 ppm (corresponding to a dry weight difference of 0.29 g) with ap-value of 0.021. This suggests that the deregulation of the nif genes engineered in the Hs12 is improving the in planta phenotype under the conditions tested.

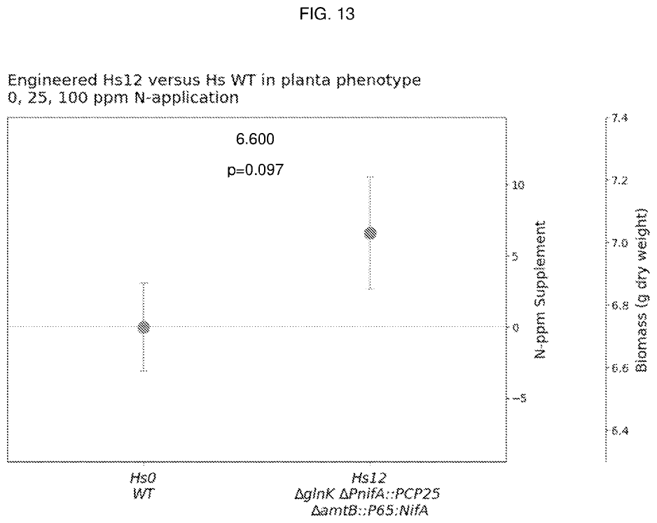

To test the engineered deregulation of the N-fix genes by exogenous nitrogen strain Hs12, the performances at three different nitrogen doses were evaluated in planta (FIG. 13). The effect of microbial treatment was assessed by a mixed model and mean effect was aggregated across nitrogen levels to improve statistical significance. A single effect was estimated with standard error that accounted for additional variation due to multiple nitrogen levels. Results obtained for this experiment are summarized in Table 6 and FIG. 13.

TABLE 6
Summary of Hs12 at Varied ppm N-application GH Experiment.

| | | Biomass | Biomass | | Std | |
|---|---|---|---|---|---|---|
| | | (g dry | difference | N-ppm | Err | p- |
| Strain | Genotype | weight) | (g) | supplemented | (g) | value |
| Hs0 | WT | 6.73 | — | — | — | — |
| Hs12 | ΔglnK, | 7.03 | 0.3 | 6.6 | 0.18 | 0.097 |
| | PnifA::PCP25, | | | | | |
| | amtB::P65:nifA | | | | | |

### (vii) Variants

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 10⁵-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically significant degree.

Variants of the protein, nucleic acid, and gene sequences also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including (but not limited to) those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, NY. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×denhardt's solution, 10% dextran sulfate, and 20 μg/mL denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH₂PO₄; 0.02M EDTA, pH 7.4), 0.5% SDS, 30% formamide, 100 g/mL salmon sperm blocking DNA; followed by washes at 50° C. with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

### (viii) Closing Paragraphs

Each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability of a genetically modified Hs microbe of the disclosure to reduce the amount of nitrogen application for an agricultural plant when the genetically modified Hs microbe is associated with the plant.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e., denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

1. A genetically modified Herbaspirillum seropedicae (Hs) microbe comprising:
(a) downregulation of endogenous glnK gene encoding P11-like nitrogen regulatory protein;
(b) a heterologous nifA gene encoding transcriptional activator NifA operably linked to a first heterologous constitutive promoter;
(c) downregulation of endogenous amtB gene encoding ammonium transporter AmtB; and
(d) endogenous nifA gene operably linked to a second heterologous constitutive promoter, wherein the second heterologous constitutive promoter replaces the endogenous nifA promoter.

2. The genetically modified Hs microbe of claim 1, wherein the downregulation of the endogenous glnK gene comprises a deletion of the endogenous glnK gene.

3. The genetically modified Hs microbe of claim 1, wherein the downregulation of the endogenous amtB gene comprises a deletion of the endogenous amtB gene such that expression of AmtB is reduced or eliminated.

4. The genetically modified Hs microbe of claim 3, wherein the deletion of the endogenous amtB gene comprises replacement of the endogenous amtB gene with the heterologous nifA gene operably linked to the first constitutive promoter.

5. The genetically modified Hs microbe of claim 1, wherein the first heterologous or second heterologous constitutive promoter is P65, CP25, or CP32.

6. The genetically modified Hs microbe of claim 1, wherein the first heterologous constitutive promoter is P65 and the second heterologous constitutive promoter is CP25.

7. The genetically modified Hs microbe of claim 1, wherein nitrogenase activity of the genetically modified Hs microbe is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to nitrogenase activity of a control Hs microbe as measured by an acetylene reduction assay, an 15N₂ fixing assay, an 15N dilution assay, and/or an ammonia biosensor assay.

8. The genetically modified Hs microbe of claim 1, wherein ammonia secretion from the genetically modified Hs microbe is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to ammonia secretion of a control Hs microbe as measured by an ammonia biosensor assay.

9. The genetically modified Hs microbe of claim 7, wherein the control Hs microbe is an Hs microbe that is not genetically modified.

10-11. (canceled)

12. A formulation comprising a genetically modified Hs microbe of claim 1 and a carrier.

13. The formulation of claim 12, wherein the formulation comprises a stabilizer, a surfactant, an adherent, a fungicide, a nematicide, an insecticide, an herbicide, a virucide, a nutrient, or a combination thereof.

14-19. (canceled)

20. A method of reducing nitrogen fertilizer application, comprising inoculating a plant or plant part with a formulation of claim 12, and growing a plant from the inoculated plant or plant part, wherein application of nitrogen to the grown plant is reduced as compared to application of nitrogen to a reference agricultural plant grown from a plant or plant part that has not been contacted with the formulation.

21. The method of claim 20, wherein the reduction in the application of nitrogen (N) is measured as N replacement per application, and wherein the N replacement is at least 5 ppm of N, at least 6 ppm of N, at least 7 ppm of N, at least 8 ppm of N, at least 9 ppm of N, at least 10 ppm of N, at least 11 ppm of N, at least 12 ppm of N, at least 13 ppm of N, at least 14 ppm of N, at least 15 ppm of N, at least 16 ppm of N, or greater.

22-23. (canceled)

24. A composition comprising a plant or plant part and a genetically modified Hs microbe of claim 1.

25. The composition of claim 24, wherein the plant part is a seed and the genetically modified Hs microbe is part of a seed coating.

26. The composition of claim 24, wherein the genetically modified Hs microbe is present in an amount of 105-109 CFU per plant or plant part.

27. (canceled)

28. The composition of claim 24, wherein the plant or plant part is from: maize, wheat, soybean, barley, millet, rice, turfgrass, cotton, canola, rapeseed, alfalfa, tomato, sugarbeet, oats, rye, sorghum, almond, walnut, apple, peanut, strawberry, lettuce, orange, potato, banana, sugarcane, cassava, mango, guava, palm, onions, olives, peppers, tea, yams, cacao, sunflower, asparagus, carrot, coconut, lemon, lime, watermelon, cabbage, cucumber, and grape.

29-32. (canceled)

33. A genetic construct comprising 5′ to 3′: (a) an upstream (US) homology arm comprising sequence homologous to sequence 5′ of the start codon of an endogenous Hs glnK coding sequence; and (b) a downstream (DS) homology arm comprising sequence homologous to sequence 3′ of the stop codon of the endogenous Hs glnK coding sequence.

34. The genetic construct of claim 33, wherein the genetic construct comprises a sequence having at least 90% sequence identity to SEQ ID NO: 1 or comprises a sequence as set forth in SEQ ID NO: 1.

35. A genetic construct comprising 5′ to 3′:
(a) an upstream (US) homology arm comprising sequence homologous to sequence 5′ of the start codon of an endogenous Hs amtB coding sequence;
(b) a P65 promoter;
(c) an nifA coding sequence; and
(d) a downstream (DS) homology arm comprising sequence homologous to sequence 3′ of the stop codon of the endogenous Hs amtB coding sequence.

36. The genetic construct of claim 35, wherein the genetic construct comprises a sequence having at least 90% sequence identity to SEQ ID NO: 2 or comprises a sequence as set forth in SEQ ID NO: 2.

37-43. (canceled)

44. A cell comprising the genetic construct of claim 33.


SEQUENCE LISTING (19 sequences)

SEQ ID NO: 1 - DNA, 4001 bp, synthetic construct
  misc_feature 1..4001: note = glnK upstream homology arm - glnK downstream homology arm sequence cassette
  source 1..4001: mol_type = other DNA
tctcgtcggcattggcggcgggcaggatgaaggcgcgccgccgttcgtcggcgctggcgccgtgctgcatggcgcaggtcatcgccagggcgccgcggatgggcaacaactggccggagagggatagctcgccggcgtattcgtagtcgtccagccggtcgccggggatctgctgggaggcggccaggatgcccagggcgatggccaggtcgaagcggccggattccttggggaggtcggcaggcgcgaggttgacggtgatgcgccgcgccgggaattcaaagcgggcgttctgcaatgccgcccggacgcggtcacgggcttccttcacctcggcctcgggcaggccgacgatggcgaaggagggcaggccattggccagatgcacctccaccgtgaccggtggggcgttcatgccggccagggcgcggctgcgcaggaccgccaggctcatcgcgatgggcctggctgcctgttacctatatagagaagagagtgtgatcgaagtgaaagtaggtgcgccaggatcatggagtctgtttttgctgggcttccagctcggccacacgggcctccagggcttccaggcgggcgcgggtcttggccagcacctgagcctggatgtcgaattcctcgcgcgtgaccaggtcgagcttggcaaagccctggctgagcatggccttgacgttcttttcgatgtccttggccggggagttttcgattgccttgttgatcttggattgcaggtcttcaaaaaacgggggcttgtccatgtcggatctccttggcagataagctgtagctggaatgcaagtttagcggtttcctgcccatcctcaaaccgccttgcctggatgagaccgcatttttaccgccaagtgcggcctgtgaagttttttccttcaggaaatgcgccgtcttggtgatccggaacgcccctgctggccgcgcaccaaaatgtgattgccggaatggaagcagggttaacaaaaaacaagcaatatcgcaccaatcacgtgcattttcccttggtctgcactattttgaactgaagttagaaaaaaaacgtgaccaatctccggtttcacctaaaaacacaagcctgccttgccgaaagaacatggcacggctcctgctacagggtttgagcaaacacaaaaaacgacttttattgttcaaacacaaaccagagggaaagtcatgaagaaactgattcttgcaacagccatcgccgcatccttcgtttctaccgtggcccgtgcagaagacgccaaaccagataatgagttcagcttcaacgccgctgtcgtgagcgactaccgctaccgcggcctgacccagacccgcttcgacccggccctgcaagtcggtgctgactacacccacaaccccaccggcctgtacgccggcacctggctgaccaacatcaagtggatcaaggacgctggcgccgtggccagcccgccggtcaacaccaagggcgacatcgagtgggacatctatggcggcaagcgcggcgacatcggcggcggcttcacctatgacgtcggcggcctgtactactactacccgggcaacaccctggccgacagcggcgccaagaacgccaacaccttcgaactgtacggccaggtcggctacggtccgggctacttcaagtattcgcaatcgctgaccaacctgttcggcgcagccaacagcaagtacagccagtacttcgacctgggcgccaacatcgagacctactacggcgtcatcctgggcctgcacgtgggtcaccagaacgtgcgcggcaaccccaaccagttcagctacaccgactggaagatcggcgtgtccaagaccttcgacgatctggccggcatcaccctgggcctggcctatgtcggcaccgacctcaaggatggcgtgaccaccacgccggcttcggatggcctgaagaacgtcggcaagaacggcttcgttttctccctcagcaaaactttctaacaagaggttcgtaaaaggggaactctaatgagcatgaagaaaatgttttccggcgcgctcgccattggcgcgctgctgtttgcagtcagttttactgctccggcccaggcgcaggatgcgcccaagcctgaagctgcagcggctgccgcagcggccccggctgctgctcccgctgcacccaaggcggaagctgccgctcctgcggccgctgccccggccgcaccggctgccgctgccgccgctcccgcaccggtccccaacaagggcgataccgcctggatgctggtctgcaccgccttcgtcatcctcatgaccttgccgggcctgggcctgttctacggcggcctggtgcgcagcaagaacatgctgtcggtgctgatgcagtgcttcatgatcttctcgctgatctacatcctgtggttcgtctacggctacagcatcgcgttcaccgagggcaatgccttcttcggtggcttcgatcgcctgttcctgcacggtctgacgggtgactccaacaccggcaccttcagcaagggcgtggtcattccggagctggtcttcgtcgccttccagggtgcgttcgcggccatcactgtggcgctgatcattggcgccttcgccgaacgcgtgaagttctcggccgtgatcctgttctccatcctgtggttcaccttctcctacctgccgatggcccacatggtctggttctggccgggtccggacgccttcaccgacgctgccgctggtgaaaaggccaccgccatctcgggctggctgttccagaagggcgcgctggactacgcaggcggcacggtggtgcacatcaacgccgctatcgccggcctggtcggttcctatgtgatcggcaagcgtatcggtttcggcaaggaagccttcaagccgcatagcctgaccctgaccatggtcggcgcctcgctgctgtggttcggctggttcggcttcaacgccggctcgggtctggaagccaacggcactgctgccctggccttcgtcaacaccatcctggcaacggcggctgctgtgctgtcctggagcttcggcgaatggatcggcaagggcaagccctcgatgctgggcgccgcttccggcgcggtggctggtctggtggcgatcaccccggcggctggtttcgtgggcgtcatcggcgccgtggtcatgggcctgatcgctggcctgctgtgcctgtggggcgtgagcggcctgaagaagatcctgggttccgacgatgcgctggacgtgttcggcgtgcacggcgtgggcggcatcctgggcgccatcctgaccggcgtgttcgcagcccccagcctgggcggcaccggcatctacgactacgtggccaacgctgttgcgcctgactactccatcggcggccaggtctggatccagttccagggcgtgctgaccaccgtggtcctgtcgggtgtggtctccttcatcgcctacaagatcgttgacgtcgtcatcggcctgcgtgtgaccgaggaagaagaacgcgaaggcctggatatctccagccacggcgaatcggcttaccactaagcctctgccgtactgatcgactgagtctgcagtattccttcggggccggcgacggcctctttcaaaggcgccttcgggcgccttttttcattgcctggatgcaagctgcttttttctgtggcattcgcgcgtcgccccggataattcccgactatttgagtcggactctttaaaatccctccatcatcgccatctgaacactcccagcgacgctttcgcagcgggcatcgccagggccggcctttgccggaatggcaacagtggcttgtgtcgcgtcgcaacacgatgtcaggaatccgctaaaatcgatcctcgctgccggcatttacatatcgttaaggtctcttatggtcccgcatctcgtcaccgctctgaatggtcctctgctggagctggagaagaaaatcctgggcgccacgccggccatcgaacgctggttccgcatggaatgg

SEQ ID NO: 2 - DNA, 5860 bp, synthetic construct
  misc_feature 1..5860: note = amtB upstream homology arm - P65 promoter - Hs NifA CDS - amtB downstream homology arm sequence cassette
  source 1..5860: mol_type = other DNA
ggcgaaggagggcaggccattggccagatgcacctccaccgtgaccggtggggcgttcatgccggccagggcgcggctgcgcaggaccgccaggctcatcgcgatgggcctggctgcctgttacctatatagagaagagagtgtgatcgaagtgaaagtaggtgcgccaggatcatggagtctgtttttgctgggcttccagctcggccacacgggcctccagggcttccaggcgggcgcgggtcttggccagcacctgagcctggatgtcgaattcctcgcgcgtgaccaggtcgagcttggcaaagccctggctgagcatggccttgacgttcttttcgatgtccttggccggggagttttcgattgccttgttgatcttggattgcaggtcttcaaaaaacgggggcttgtccatgtcggatctccttggcagataagctgtagctggaatgcaagtttagcggtttcctgcccatcctcaaaccgccttgcctggatgagaccgcatttttaccgccaagtgcggcctgtgaagttttttccttcaggaaatgcgccgtcttggtgatccggaacgcccctgctggccgcgcaccaaaatgtgattgccggaatggaagcagggttaacaaaaaacaagcaatatcgcaccaatcacgtgcattttcccttggtctgcactattttgaactgaagttagaaaaaaaacgtgaccaatctccggtttcacctaaaaacacaagcctgccttgccgaaagaacatggcacggctcctgctacagggtttgagcaaacacaaaaaacgacttttattgttcaaacacaaaccagagggaaagtcatgaagaaactgattcttgcaacagccatcgccgcatccttcgtttctaccgtggcccgtgcagaagacgccaaaccagataatgagttcagcttcaacgccgctgtcgtgagcgactaccgctaccgcggcctgacccagacccgcttcgacccggccctgcaagtcggtgctgactacacccacaaccccaccggcctgtacgccggcacctggctgaccaacatcaagtggatcaaggacgctggcgccgtggccagcccgccggtcaacaccaagggcgacatcgagtgggacatctatggcggcaagcgcggcgacatcggcggcggcttcacctatgacgtcggcggcctgtactactactacccgggcaacaccctggccgacagcggcgccaagaacgccaacaccttcgaactgtacggccaggtcggctacggtccgggctacttcaagtattcgcaatcgctgaccaacctgttcggcgcagccaacagcaagtacagccagtacttcgacctgggcgccaacatcgagacctactacggcgtcatcctgggcctgcacgtgggtcaccagaacgtgcgcggcaaccccaaccagttcagctacaccgactggaagatcggcgtgtccaagaccttcgacgatctggccggcatcaccctgggcctggcctatgtcggcaccgacctcaaggatggcgtgaccaccacgccggcttcggatggcctgaagaacgtcggcaagaacggcttcgttttctccctcagcaaaactttctaacaagaggttcgtatgaaactgatcacagcgatcatcaaaccgttcaagctggacgaggtgcgtgaagccctctccgcgatcggggtgcaaggcatcaccgtgactgaagtcaagggcttcggtcgtcagaagggccataccgagctgtaccgcggcgcggaatacgtggtcgatttcctgcccaagaccaagatcgaggcggcggtggacgacgccatcgtcgaacgcgcgctcgaagccatcgagaccgctgcccgcaccggcaagatcggcgacggcaagatcttcgtccaggacctgctggacgtgatccgtatccgcaccggcgagaccggcaaagacgctctgtaaaaaaggggaactctaggccgccgtagtgtccctgcccggctcgtgggacggcgcgcatcccggtcagccgggatgtttcacgtgaaacatccgttcgcatcctttctccaaaaaaccctcttgacttggagtccgctcgaagccctatgatgataggcgaacccatcgagaggagcatctatggccactattctcgacgaccgcagtgtgaatttagaactcgtaacgatttacgagatcagcaagatattgggttcatcgctggatctatccaagaccttgcgcgaagtcctcaacgtcctctccgcccacctggaaacgaaacgggtattgctgagtctgatgcaggactccggtgaattgcaactggtcagcgccatcggcctgagttacgaagaattccagagtggcagataccgggtcggcgagggcatcaccggcaagatcttccagacggaaactcccatcgtggtgcgcgacttggcccaggagccgctgttcctggctcgcaccagcccgcgccagtcgcaagacggggaagtcatttcctttgtgggcgtgccgatcaaggctgcccgtgaaatgctgggtgtgttgtgcgtttttcgggatggacaatcgccctcgcgtagtgtcgaccatgaggtgcgcctgctgaccatggtggccaacctgatcggacaaaccgtccggctgtatcgcagcgtagcggccgagcgccagcagctgcaagaggaaaagcgccagctgtcccggcaactccagggcaagtacaagctcgataacgtaatcggcatttccaaggcgatgcaggaagtgttcgcgcaggtacaccagtccgccccctcgcgctcgaccatgctgttgcgcggcgagagcggcaccggcaaggaagtcatcgcgcgcgccatccattacctcagtccgcgcaaggatgggccattcatcaaggtcaactgtgcggccttgtcggaaaccttgctggaatccgaattgtttggccacgagaagggcgccttcaccggggcccagggtgaacgcaaggggcgtttcgaactcgcccatggcggcaccttgttcctcgacgagatcggcgagatctcgccggccttccaggccaagctgctgcgggtattgcaggagcgtgaattcgagcgggtcggtgggtcgcgttcgatcaaggtggacgtgcgtctggtcaccgccaccaaccgcgacctggagaaggccgtggccaagggcgagttccgggccgacctgtattaccgtatcaatgtggtcagcatcttcattcctccgctgcgcgaacggcgcgaggatattccctatctggtggaacacttcctggaaaaattccgtgtggagaaccagcgcgccatggtggccatgtcaccgcaggccatgaaggtcatgatgaattgctactggccgggcaatgtacgggaactggaaaattgtgtcgagcgtactgccaccatgatgcgcggcgatctgatcaccgaggtgcatttctcctgtcagcagaacaaatgcctgaccaaggtcttgcatgaacccggccagcagcaaccggtcgtggtggtgccgctggagcgcatatccgcgcatacggcgccatcttcgccgagtgggatggccaaggacaagccactggcggccgcgcccccgacctccgaacgcgaacgtcttatctgggccatggagcaatgtggctgggtgcaagccaaggcagcccgcgcactcaacatctcgccgcgccagatgggctatgcgctgcagaaattcaatatcgaggtcaagaagttctaataaatcccatcaaataaaacgaaaggctcagtcgaaagactgggcctttcgttttatctgttgttttcctctgccgtactgatcgactgagtctgcagtattccttcggggccggcgacggcctctttcaaaggcgccttcgggcgccttttttcattgcctggatgcaagctgcttttttctgtggcattcgcgcgtcgccccggataattcccgactatttgagtcggactctttaaaatccctccatcatcgccatctgaacactcccagcgacgctttcgcagcgggcatcgccagggccggcctttgccggaatggcaacagtggcttgtgtcgcgtcgcaacacgatgtcaggaatccgctaaaatcgatcctcgctgccggcatttacatatcgttaaggtctcttatggtcccgcatctcgtcaccgctctgaatggtcctctgctggagctggagaagaaaatcctgggcgccacgccggccatcgaacgctggttccgcatggaatggcaggagcacacgccgcccttctattgctcggtcgacctgcgcaacgccggcttcaagctggcgccggtcgacaccaacctgttcccgggcggcttcaacaacctttctcccgagatgctgccgctggcggtgcaggcggccatggccgccatcgagaaatactgtcccgatgcccgcaacctgctgctgatcccggaaaaccacaccggcaacaccttctacctgcagaacatcgcccggctgatgcagatcttccgccagaccgggctgaacgtgcgcctgggcagcctggcgccgggcgtgaacaagcccaagccgatcgaactgcccgatggcaccaccatcaccctggagccgctggagcgctcggccaacggccgccgcctgggactgaagaacttcgatccctgtacggtgctgctcaacaatgacctgtccatcggcatcccgtcggtgctggaaggcttgcatgagcagagcgtgctgccgccgttgcacgccggctgggcgatgcgccgcaagagcaaccatttcgccgcctatgatgaggtggtcaagaagtttgccaagatggtcgaggtcgatccctggatgctcaatccgctctttgccaaggtcagcgacatcgacttccaggaaagcaccggcaccgatgcgctggccgacagcgtttcttcgctgttgtccaagatccgcaagaagtacaaggaatacggtatcaaggagcagcccttcgtcttcgtcaaggccaacgccggcacctatggcatgggcatcatgacggtcaaggacgccagcgagatcaaggacctgaaccgcaagcagcgcaccggcatgctggcggccggcaaggaagggctggaggtgtcggaggtgatcgtgcaggaaggcgtgcattccttcgagcgcatcaaccaggccgtggccgagccggtggtctacatgatcgaccgctatgtggtgggcggcttctatcgcgtccacgccgaccgtgacgtcaacgagaacctcaacgcacccggcgcgcatttcgtgccgctggccttcgcccagcagcacgccctgccggacatgcatgccaagccgggcacggcggcgcccaaccgtttctacatgtatggcgtggtggcgcgattggcgctgctggcggcctcggtggaaatggaaaagaccgatcccaacccggaagtttattaatatcgggccttcgccaggctcaacaggaccctagcctcatgaaaatcgctttcctcgccgatccactggatacgttcaagacgtacaaggattccaccttcgccatgatgcgcgaagccgcgcgccgtggccacgtcctctacgccttcgaacaggccgacctggtgctggaaagcgggctggtgacggccaacgcctgcaagctcacgctcacgggcgatgccgatgactggtatcggaccgacaagccggtctcttctttcctgtccgaattcgatgcggtcatcgtgcgcaaggacccgcccttcgacatggagtacatctacacgacctacctgctggagctggccg

SEQ ID NO: 3 - DNA, 5761 bp, synthetic construct
  misc_feature 1..5761: note = amtB upstream homology arm - CP32 promoter - Hs NifA CDS - amtB downstream homology arm sequence cassette
  source 1..5761: mol_type = other DNA
ggcgaaggagggcaggccattggccagatgcacctccaccgtgaccggtggggcgttcatgccggccagggcgcggctgcgcaggaccgccaggctcatcgcgatgggcctggctgcctgttacctatatagagaagagagtgtgatcgaagtgaaagtaggtgcgccaggatcatggagtctgtttttgctgggcttccagctcggccacacgggcctccagggcttccaggcgggcgcgggtcttggccagcacctgagcctggatgtcgaattcctcgcgcgtgaccaggtcgagcttggcaaagccctggctgagcatggccttgacgttcttttcgatgtccttggccggggagttttcgattgccttgttgatcttggattgcaggtcttcaaaaaacgggggcttgtccatgtcggatctccttggcagataagctgtagctggaatgcaagtttagcggtttcctgcccatcctcaaaccgccttgcctggatgagaccgcatttttaccgccaagtgcggcctgtgaagttttttccttcaggaaatgcgccgtcttggtgatccggaacgcccctgctggccgcgcaccaaaatgtgattgccggaatggaagcagggttaacaaaaaacaagcaatatcgcaccaatcacgtgcattttcccttggtctgcactattttgaactgaagttagaaaaaaaacgtgaccaatctccggtttcacctaaaaacacaagcctgccttgccgaaagaacatggcacggctcctgctacagggtttgagcaaacacaaaaaacgacttttattgttcaaacacaaaccagagggaaagtcatgaagaaactgattcttgcaacagccatcgccgcatccttcgtttctaccgtggcccgtgcagaagacgccaaaccagataatgagttcagcttcaacgccgctgtcgtgagcgactaccgctaccgcggcctgacccagacccgcttcgacccggccctgcaagtcggtgctgactacacccacaaccccaccggcctgtacgccggcacctggctgaccaacatcaagtggatcaaggacgctggcgccgtggccagcccgccggtcaacaccaagggcgacatcgagtgggacatctatggcggcaagcgcggcgacatcggcggcggcttcacctatgacgtcggcggcctgtactactactacccgggcaacaccctggccgacagcggcgccaagaacgccaacaccttcgaactgtacggccaggtcggctacggtccgggctacttcaagtattcgcaatcgctgaccaacctgttcggcgcagccaacagcaagtacagccagtacttcgacctgggcgccaacatcgagacctactacggcgtcatcctgggcctgcacgtgggtcaccagaacgtgcgcggcaaccccaaccagttcagctacaccgactggaagatcggcgtgtccaagaccttcgacgatctggccggcatcaccctgggcctggcctatgtcggcaccgacctcaaggatggcgtgaccaccacgccggcttcggatggcctgaagaacgtcggcaagaacggcttcgttttctccctcagcaaaactttctaacaagaggttcgtatgaaactgatcacagcgatcatcaaaccgttcaagctggacgaggtgcgtgaagccctctccgcgatcggggtgcaaggcatcaccgtgactgaagtcaagggcttcggtcgtcagaagggccataccgagctgtaccgcggcgcggaatacgtggtcgatttcctgcccaagaccaagatcgaggcggcggtggacgacgccatcgtcgaacgcgcgctcgaagccatcgagaccgctgcccgcaccggcaagatcggcgacggcaagatcttcgtccaggacctgctggacgtgatccgtatccgcaccggcgagaccggcaaagacgctctgtaaaaaaggggaactctacatacgggagtttattcttgacatattgccggtgtgttggtataataacttaagtcaataaaggcatataaaaggaggttaataacatgaaagttaaagtaaaacatcttaatcatgcggtggagggtttctaatggccactattctcgacgaccgcagtgtgaatttagaactcgtaacgatttacgagatcagcaagatattgggttcatcgctggatctatccaagaccttgcgcgaagtcctcaacgtcctctccgcccacctggaaacgaaacgggtattgctgagtctgatgcaggactccggtgaattgcaactggtcagcgccatcggcctgagttacgaagaattccagagtggcagataccgggtcggcgagggcatcaccggcaagatcttccagacggaaactcccatcgtggtgcgcgacttggcccaggagccgctgttcctggctcgcaccagcccgcgccagtcgcaagacggggaagtcatttcctttgtgggcgtgccgatcaaggctgcccgtgaaatgctgggtgtgttgtgcgtttttcgggatggacaatcgccctcgcgtagtgtcgaccatgaggtgcgcctgctgaccatggtggccaacctgatcggacaaaccgtccggctgtatcgcagcgtagcggccgagcgccagcagctgcaagaggaaaagcgccagctgtcccggcaactccagggcaagtacaagctcgataacgtaatcggcatttccaaggcgatgcaggaagtgttcgcgcaggtacaccagtccgccccctcgcgctcgaccatgctgttgcgcggcgagagcggcaccggcaaggaagtcatcgcgcgcgccatccattacctcagtccgcgcaaggatgggccattcatcaaggtcaactgtgcggccttgtcggaaaccttgctggaatccgaattgtttggccacgagaagggcgccttcaccggggcccagggtgaacgcaaggggcgtttcgaactcgcccatggcggcaccttgttcctcgacgagatcggcgagatctcgccggccttccaggccaagctgctgcgggtattgcaggagcgtgaattcgagcgggtcggtgggtcgcgttcgatcaaggtggacgtgcgtctggtcaccgccaccaaccgcgacctggagaaggccgtggccaagggcgagttccgggccgacctgtattaccgtatcaatgtggtcagcatcttcattcctccgctgcgcgaacggcgcgaggatattccctatctggtggaacacttcctggaaaaattccgtgtggagaaccagcgcgccatggtggccatgtcaccgcaggccatgaaggtcatgatgaattgctactggccgggcaatgtacgggaactggaaaattgtgtcgagcgtactgccaccatgatgcgcggcgatctgatcaccgaggtgcatttctcctgtcagcagaacaaatgcctgaccaaggtcttgcatgaacccggccagcagcaaccggtcgtggtggtgccgctggagcgcatatccgcgcatacggcgccatcttcgccgagtgggatggccaaggacaagccactggcggccgcgcccccgacctccgaacgcgaacgtcttatctgggccatggagcaatgtggctgggtgcaagccaaggcagcccgcgcactcaacatctcgccgcgccagatgggctatgcgctgcagaaattcaatatcgaggtcaagaagttctgacctctgccgtactgatcgactgagtctgcagtattccttcggggccggcgacggcctctttcaaaggcgccttcgggcgccttttttcattgcctggatgcaagctgcttttttctgtggcattcgcgcgtcgccccggataattcccgactatttgagtcggactctttaaaatccctccatcatcgccatctgaacactcccagcgacgctttcgcagcgggcatcgccagggccggcctttgccggaatggcaacagtggcttgtgtcgcgtcgcaacacgatgtcaggaatccgctaaaatcgatcctcgctgccggcatttacatatcgttaaggtctcttatggtcccgcatctcgtcaccgctctgaatggtcctctgctggagctggagaagaaaatcctgggcgccacgccggccatcgaacgctggttccgcatggaatggcaggagcacacgccgcccttctattgctcggtcgacctgcgcaacgccggcttcaagctggcgccggtcgacaccaacctgttcccgggcggcttcaacaacctttctcccgagatgctgccgctggcggtgcaggcggccatggccgccatcgagaaatactgtcccgatgcccgcaacctgctgctgatcccggaaaaccacaccggcaacaccttctacctgcagaacatcgcccggctgatgcagatcttccgccagaccgggctgaacgtgcgcctgggcagcctggcgccgggcgtgaacaagcccaagccgatcgaactgcccgatggcaccaccatcaccctggagccgctggagcgctcggccaacggccgccgcctgggactgaagaacttcgatccctgtacggtgctgctcaacaatgacctgtccatcggcatcccgtcggtgctggaaggcttgcatgagcagagcgtgctgccgccgttgcacgccggctgggcgatgcgccgcaagagcaaccatttcgccgcctatgatgaggtggtcaagaagtttgccaagatggtcgaggtcgatccctggatgctcaatccgctctttgccaaggtcagcgacatcgacttccaggaaagcaccggcaccgatgcgctggccgacagcgtttcttcgctgttgtccaagatccgcaagaagtacaaggaatacggtatcaaggagcagcccttcgtcttcgtcaaggccaacgccggcacctatggcatgggcatcatgacggtcaaggacgccagcgagatcaaggacctgaaccgcaagcagcgcaccggcatgctggcggccggcaaggaagggctggaggtgtcggaggtgatcgtgcaggaaggcgtgcattccttcgagcgcatcaaccaggccgtggccgagccggtggtctacatgatcgaccgctatgtggtgggcggcttctatcgcgtccacgccgaccgtgacgtcaacgagaacctcaacgcacccggcgcgcatttcgtgccgctggccttcgcccagcagcacgccctgccggacatgcatgccaagccgggcacggcggcgcccaaccgtttctacatgtatggcgtggtggcgcgattggcgctgctggcggcctcggtggaaatggaaaagaccgatcccaacccggaagtttattaatatcgggccttcgccaggctcaacaggaccctagcctcatgaaaatcgctttcctcgccgatccactggatacgttcaagacgtacaaggattccaccttcgccatgatgcgcgaagccgcgcgccgtggccacgtcctctacgccttcgaacaggccgacctggtgctggaaagcgggctggtgacggccaacgcctgcaagctcacgctcacgggcgatgccgatgactggtatcggaccgacaagccggtctcttctttcctgtccgaattcgatgcggtcatcgtgcgcaaggacccgcccttcgacatggagtacatctacacgacctacctgctggagctggccg

SEQ ID NO: 4 - DNA, 3999 bp, synthetic construct
  misc_feature 1..3999: note = amtB upstream homology arm - amtB downstream homology arm sequence cassette
  source 1..3999: mol_type = other DNA
ggcgaaggagggcaggccattggccagatgcacctccaccgtgaccggtggggcgttcatgccggccagggcgcggctgcgcaggaccgccaggctcatcgcgatgggcctggctgcctgttacctatatagagaagagagtgtgatcgaagtgaaagtaggtgcgccaggatcatggagtctgtttttgctgggcttccagctcggccacacgggcctccagggcttccaggcgggcgcgggtcttggccagcacctgagcctggatgtcgaattcctcgcgcgtgaccaggtcgagcttggcaaagccctggctgagcatggccttgacgttcttttcgatgtccttggccggggagttttcgattgccttgttgatcttggattgcaggtcttcaaaaaacgggggcttgtccatgtcggatctccttggcagataagctgtagctggaatgcaagtttagcggtttcctgcccatcctcaaaccgccttgcctggatgagaccgcatttttaccgccaagtgcggcctgtgaagttttttccttcaggaaatgcgccgtcttggtgatccggaacgcccctgctggccgcgcaccaaaatgtgattgccggaatggaagcagggttaacaaaaaacaagcaatatcgcaccaatcacgtgcattttcccttggtctgcactattttgaactgaagttagaaaaaaaacgtgaccaatctccggtttcacctaaaaacacaagcctgccttgccgaaagaacatggcacggctcctgctacagggtttgagcaaacacaaaaaacgacttttattgttcaaacacaaaccagagggaaagtcatgaagaaactgattcttgcaacagccatcgccgcatccttcgtttctaccgtggcccgtgcagaagacgccaaaccagataatgagttcagcttcaacgccgctgtcgtgagcgactaccgctaccgcggcctgacccagacccgcttcgacccggccctgcaagtcggtgctgactacacccacaaccccaccggcctgtacgccggcacctggctgaccaacatcaagtggatcaaggacgctggcgccgtggccagcccgccggtcaacaccaagggcgacatcgagtgggacatctatggcggcaagcgcggcgacatcggcggcggcttcacctatgacgtcggcggcctgtactactactacccgggcaacaccctggccgacagcggcgccaagaacgccaacaccttcgaactgtacggccaggtcggctacggtccgggctacttcaagtattcgcaatcgctgaccaacctgttcggcgcagccaacagcaagtacagccagtacttcgacctgggcgccaacatcgagacctactacggcgtcatcctgggcctgcacgtgggtcaccagaacgtgcgcggcaaccccaaccagttcagctacaccgactggaagatcggcgtgtccaagaccttcgacgatctggccggcatcaccctgggcctggcctatgtcggcaccgacctcaaggatggcgtgaccaccacgccggcttcggatggcctgaagaacgtcggcaagaacggcttcgttttctccctcagcaaaactttctaacaagaggttcgtatgaaactgatcacagcgatcatcaaaccgttcaagctggacgaggtgcgtgaagccctctccgcgatcggggtgcaaggcatcaccgtgactgaagtcaagggcttcggtcgtcagaagggccataccgagctgtaccgcggcgcggaatacgtggtcgatttcctgcccaagaccaagatcgaggcggcggtggacgacgccatcgtcgaacgcgcgctcgaagccatcgagaccgctgcccgcaccggcaagatcggcgacggcaagatcttcgtccaggacctgctggacgtgatccgtatccgcaccggcgagaccggcaaagacgctctgtaaaaaaggggaactctacctctgccgtactgatcgactgagtctgcagtattccttcggggccggcgacggcctctttcaaaggcgccttcgggcgccttttttcattgcctggatgcaagctgcttttttctgtggcattcgcgcgtcgccccggataattcccgactatttgagtcggactctttaaaatccctccatcatcgccatctgaacactcccagcgacgctttcgcagcgggcatcgccagggccggcctttgccggaatggcaacagtggcttgtgtcgcgtcgcaacacgatgtcaggaatccgctaaaatcgatcctcgctgccggcatttacatatcgttaaggtctcttatggtcccgcatctcgtcaccgctctgaatggtcctctgctggagctggagaagaaaatcctgggcgccacgccggccatcgaacgctggttccgcatggaatggcaggagcacacgccgcccttctattgctcggtcgacctgcgcaacgccggcttcaagctggcgccggtcgacaccaacctgttcccgggcggcttcaacaacctttctcccgagatgctgccgctggcggtgcaggcggccatggccgccatcgagaaatactgtcccgatgcccgcaacctgctgctgatcccggaaaaccacaccggcaacaccttctacctgcagaacatcgcccggctgatgcagatcttccgccagaccgggctgaacgtgcgcctgggcagcctggcgccgggcgtgaacaagcccaagccgatcgaactgcccgatggcaccaccatcaccctggagccgctggagcgctcggccaacggccgccgcctgggactgaagaacttcgatccctgtacggtgctgctcaacaatgacctgtccatcggcatcccgtcggtgctggaaggcttgcatgagcagagcgtgctgccgccgttgcacgccggctgggcgatgcgccgcaagagcaaccatttcgccgcctatgatgaggtggtcaagaagtttgccaagatggtcgaggtcgatccctggatgctcaatccgctctttgccaaggtcagcgacatcgacttccaggaaagcaccggcaccgatgcgctggccgacagcgtttcttcgctgttgtccaagatccgcaagaagtacaaggaatacggtatcaaggagcagcccttcgtcttcgtcaaggccaacgccggcacctatggcatgggcatcatgacggtcaaggacgccagcgagatcaaggacctgaaccgcaagcagcgcaccggcatgctggcggccggcaaggaagggctggaggtgtcggaggtgatcgtgcaggaaggcgtgcattccttcgagcgcatcaaccaggccgtggccgagccggtggtctacatgatcgaccgctatgtggtgggcggcttctatcgcgtccacgccgaccgtgacgtcaacgagaacctcaacgcacccggcgcgcatttcgtgccgctggccttcgcccagcagcacgccctgccggacatgcatgccaagccgggcacggcggcgcccaaccgtttctacatgtatggcgtggtggcgcgattggcgctgctggcggcctcggtggaaatggaaaagaccgatcccaacccggaagtttattaatatcgggccttcgccaggctcaacaggaccctagcctcatgaaaatcgctttcctcgccgatccactggatacgttcaagacgtacaaggattccaccttcgccatgatgcgcgaagccgcgcgccgtggccacgtcctctacgccttcgaacaggccgacctggtgctggaaagcgggctggtgacggccaacgcctgcaagctcacgctcacgggcgatgccgatgactggtatcggaccgacaagccggtctcttctttcctgtccgaattcgatgcggtcatcgtgcgcaaggacccgcccttcgacatggagtacatctacacgacctacctgctggagctggccg

SEQ ID NO: 5 - DNA, 4285 bp, synthetic construct
  misc_feature 1..4285: note = Hs NifA promoter upstream homology arm - CP25 promoter - Hs NifA promoter downstream homology arm sequence cassette
  source 1..4285: mol_type = other DNA
gcgatggtgacggtgacttgcgtagtgacagggccggtatcgatcgcactgatgaccccggcgaattgattacgtgcgctggttttcattgacattctcctgagcaggccagccaaggctggcagttgattaagccatgtctcctgccgatgcaggaattcgagattgcgccgttgcagctcgcgccacacggccagcagttccaccgcggcgggcgtgagccgggtgccgccgccgcctttgccgccggtgaccgtttcaatcaatgcgccattgaccaggttgccggctgactccagcagcatccatgcgcccttgtagctgtagcccgccgtactggccgcgcgattgatcgagccggtttgcgcgatggcgtcgagcagggcaaagaagcgctggtccaggcgatgcaccagtttcagttcgctgctcaacaaggtggtatcggtggcaggcaaggtactcatgtccttgccgatggctgcagtcccagcgcgcccgccagcaagctcagggctgcgcgcgcttgctggggatcgtggctggcaggcttgagcagcaggcgatgcggccccagtttttcttgcagctggccgatgcgtgccacgcattgcgccgggtcgccgatgatggcgttggccaggatgacttgatcggaggcatcggcctggaacatgggcggggtgccaccgggcgcgaattgcacttgcatcatgcgcggatacgcacgcacacctgccagaccatgggcaagcgccttgtgcgcctcgggttcgcaatggaaaaagcgggcgatggcaaacgggaagtgaccgcgctgcgcacgctgtgcagccacccgcgtgagcacctgctcaagatcggcggagggtgtggccatcaatccgtaatcctggcgtgcggccagatcaagcgaatcttcgctcatgctggccagccataccgggatgggacgttgcagcggctgcggttgcaactgcactctgtcgtagtgaaaaaatcgtccctcggtactgaatggcacgccttgccatgcgcgctggatgacgtccagtgcttcaatcatgcgtgcgcgcgcctgatcggcaccgctcaatccggcgtgacgatattgctcgggaaacgggccgccgcgtgccacgccgaattccatccggccgttgctgagcaggtccagcgtggcgacgtcctcggcaacccgcaagggatcattgagcgccagcaacgaagcgcccgtaccgaggcggatgcgcgaagtgcgtgcggcgatctgggccagcagcaccatcaaggcgctgccgatggcgaagctgctgtaatggtgctcggcgacccagacatcgtgatagcccagggcctctgccacgacggcatgatcgacggtctgcaacagcgcatcgcgtgcgttgccatggggattttcgacattgaggaagacgccgtagcgttgcgggtaatccggcatggcaggttcacgggaaaaagagttggacatcgctcagaccggccgcatcaagaaacagatgcggggatgcgatgccggatggcgcaggtcatgattcggatcttcccgatggccggatcagatccgctatcggcggcagcatgtcggtggcgggcgagtggaaccagctgcgtaccagctcataggggtagcgccgcggcggttcgacgcgcgcatgacgtaccagcagatgaatcgacaagggttcggcccaggcccagaagtcggcccgcagcgcgggcgggaaaccgatatcgtcaaaggtatgccaaagctgtaccagccacagttcgcgcccagcttcgtccaatagcaacggcaagcctgcgcccgcttgcagatgggcatggcgttggctgtagtagagcggaccgccgcatgactccaccacgaagtcggcgatgcgcgagctgacgcaatcaaaacaattgtgcgaacgtgcgaacagcggggaatggcgcaggcgccgcatatgatgcagcaccagtttgcgcaagccggattcatcacacagcgccagcaaccggtgcgccggttgaggtaattgtggcggctcacatatcacctgttgcgtgccgcgcgccatcgccgggccggcgaccggccagttcagggtcagataacatgcctgggagcagggcgcccactttggcagtttattcttgacatgtagtgagggggctggtataatcacataagtcaataaaggcatataaaaggaggttaataacatgaaagttaaagtaaaacatcttaatcatgcggtggagggtttctaatgatggccactattctcgacgaccgcagtgtgaatttagaactcgtaacgatttacgagatcagcaagatattgggttcatcgctggatctatccaagaccttgcgcgaagtcctcaacgtcctctccgcccacctggaaacgaaacgggtattgctgagtctgatgcaggactccggtgaattgcaactggtcagcgccatcggcctgagttacgaagaattccagagtggcagataccgggtcggcgagggcatcaccggcaagatcttccagacggaaactcccatcgtggtgcgcgacttggcccaggagccgctgttcctggctcgcaccagcccgcgccagtcgcaagacggggaagtcatttcctttgtgggcgtgccgatcaaggctgcccgtgaaatgctgggtgtgttgtgcgtttttcgggatggacaatcgccctcgcgtagtgtcgaccatgaggtgcgcctgctgaccatggtggccaacctgatcggacaaaccgtccggctgtatcgcagcgtagcggccgagcgccagcagctgcaagaggaaaagcgccagctgtcccggcaactccagggcaagtacaagctcgataacgtaatcggcatttccaaggcgatgcaggaagtgttcgcgcaggtacaccagtccgccccctcgcgctcgaccatgctgttgcgcggcgagagcggcaccggcaaggaagtcatcgcgcgcgccatccattacctcagtccgcgcaaggatgggccattcatcaaggtcaactgtgcggccttgtcggaaaccttgctggaatccgaattgtttggccacgagaagggcgccttcaccggggcccagggtgaacgcaaggggcgtttcgaactcgcccatggcggcaccttgttcctcgacgagatcggcgagatctcgccggccttccaggccaagctgctgcgggtattgcaggagcgtgaattcgagcgggtcggtgggtcgcgttcgatcaaggtggacgtgcgtctggtcaccgccaccaaccgcgacctggagaaggccgtggccaagggcgagttccgggccgacctgtattaccgtatcaatgtggtcagcatcttcattcctccgctgcgcgaacggcgcgaggatattccctatctggtggaacacttcctggaaaaattccgtgtggagaaccagcgcgccatggtggccatgtcaccgcaggccatgaaggtcatgatgaattgctactggccgggcaatgtacgggaactggaaaattgtgtcgagcgtactgccaccatgatgcgcggcgatctgatcaccgaggtgcatttctcctgtcagcagaacaaatgcctgaccaaggtcttgcatgaacccggccagcagcaaccggtcgtggtggtgccgctggagcgcatatccgcgcatacggcgccatcttcgccgagtgggatggccaaggacaagccactggcggccgcgcccccgacctccgaacgcgaacgtcttatctgggccatggagcaatgtggctgggtgcaagccaaggcagcccgcgcactcaacatctcgccgcgccagatgggctatgcgctgcagaaattcaatatcgaggtcaagaagttctgaaactcatctgcgagcgtgacgtaatacgctgccagatccaaagtctttttctcattgccgtcctcgccctgcgcgcgcggtctcttcggcgtcgcgccagccggttgtattgatggattccccggctggcacgtcctcgcattctccttctccttctccactgcgtcaggctcattgcggcgcgctcaattgcaggcgccggcccgtgctgcattgtcgtgttcatgacaagcgccacccacaatgtatcgccgattgtcgctatcctcacatcgccgtacgacgcaattctccgctatgtcattgaattgattgattattttgtctggcacggttttggctatgtcctcaggagaaggcccaggcatcac

SEQ ID NO: 6 - DNA, 3998 bp, synthetic construct
  misc_feature 1..3998: note = ntrC upstream homology arm - ntrC downstream homology arm sequence cassette
  source 1..3998: mol_type = other DNA
tgtcgctattagagtgcaatgcgcatctctagcccggtatgccatgaagacgtcgcccgacgacatcgccgaaacgcccaatatcccatccacagagatgactgcttcgccgatgcgatgccaggagcttgctgctggccgaggtgcgctgccgcaaggccatccatgccggacctccgtcatccactgcgcaggccgcctccaatgcgctctgccctttcccctcaggtcaaatcgtgaacatcagaaataccaaagtcgccactcagctcatgctgggcttttccatcctgatcctcttcatggctgcgctgggaggaacgtccctctacatcatgggcaagatcaacgcccagctggtcgacgtccagggcaactggatgcccagcgtgactaccgcgcaaaccatgctggccgagatgcgggcggtcaacctggcccagtaccgcgccctgaccgccgacaatgcgcagcgcatgcaagacgcccgcacccgcatcaccgctgccctgggacgctacaccaaggccagcgagcagtaccgccacctcatcagctcgccggccgaacagagcgcctacgatgaactgcaggccttgctggcccgctatatccagatcggcgaccagttgccgcaaagcgtggagcaaggcaaggaagaagaagccgccaacaccatgaaggacacgctctatccggtgcgtaccgccatggaagccaagatccagcagatcatcaagatcaaccaggaaggcgcggcgcaggcggtcgaacacggccaggccagctatgcgcaaagccacctcaccatcatcgccttcaccctggccgcggcactggcgggactgggcattgcgctgctgatcagccgccgcctcacgcgccagctcggaggtgaaccgggcgaggcgatgtggctgtcgggccagatcgccgccggcaacctggcggcgcggctggaactcaagagcaatgacaacggcagcctgatgcgctcgctgatgatcatgcgcgaccagctcagcggcctggtgggccagatccagcatgccagcacgtccatccacgtggctgcccaggagatcgcgcagggcaacatcggtctctcgcagcgcaccgaagaacaggccgcctcgctggaagaaaccgcctccagcatggaagaactgaccagcactgtgcaacagaatgccggtaacgccaagcaggccagcagcatggccagcgccggtgcagaagtggcgcgccggggcgaggccgagatcgccgacgtggtggcgacgatgcgcgagatgagcgtgagctcgcaccagatgtcggacatcatcagcgtcatcgaaggcatttccttccagaccaatatcctggccctcaacgctgccgtggaagcggcccgcgccggcgagaacggacgcggcttcgccgtggtcgcctcggaagtgcgcgcgctggcccagcgcagctcggccgcggccaaggaaatcaaggacctgatcgaagactcggtggggcgcatccgcaacggctcccaactggtcgaagcggccggcgcgacgattgccgagatcatgcagtccagcgtgcgcaccaccgacctgatgaaggaaattgccgcggcttccgaggaacagagcgccggcatcggccaggtcaatacggccatcgtgcagatggaccaggtcacgcagcaaaacgcggcgctggtggaagaagcggcggcagccgcgcagtccatgatgcaacaggcgcaagcgctcacagcggcggtgtcggtcttcaagctggatgatcaggccttgcgtaccctgcccggcatgcccgccgcgccccagctgggccgttcactggccacgccggcgctgtcttgacggccgccgcaatagcggcgccgaggcaatgaacaatgaacaatgaacaatgcaaatggcctggatcgcaagatccaggccatttgtttttccagccacgcgcggcctggaacccgccctgccctgtgaggcatcacgtcttcctgcgttggtcttgctgttgatgctcccgcgagccaggccgcttctggcgccctgcccgcagcccccttggatgacgggatgtcttccgtcatgtcggctttggcgcgccgcctgcggccatgctcacggcagcggtataagtattctgaaatcggtacaacctggccggctgtcgcattcgatcacgcccatgtgctgatggacgaatgtctgcgccagggtcagccccagtccgctgccaccttcccgacccgacaccagtggatagaagattctgtcctggatgtcgggggggatgcccggaccgttatcgatgatatgcaagtctagtgccagccggtatcggaccttggatagcgtcacttgacggctcacgcgagtacgcaggatgagctcgccatcgccggcgcggatgcggtcggccagcgcctgggcggcgttgtgcacgatgttgagcacggcctggatgagctgctccttgtcgccgcgaaattccgggatggacaggtcatagtcgcgcttgatggtcagcccctgggggaactcggccatgaccaggctgcgcacgcgttcacagacttcgtggatgttgacgtcgccgacgatgtgcggatgccggtgcggcgccagcaggcgatcgaccagggtctgcaggcgatcggcttccttgatgatgacctgggtatattcgcgcaattccttgaggtgccgctcgggcagctccagctccagcagctgcgctgcgccgcggatgccgcccagcggattcttgatctcgtgggccaggttgcggatcagttccttgttggcctggctctggtcgaagatgcgctcttcgcggtccagcttcaattgctggacgttttcgcgcagctccagcagcaccggcatgtcggggttgtccagcggcgtgacgatggtgtggacctgcagcggttcgcggttggcgcgttccagcaccaggtccaggcgcttgtcggcgaactggtgttcgatggcctggtagaacagggccgagagctccttgccgttcaggaagagctccgagagcttctgctgcgagagcaccttgaaggagctttccagcaggttctcggcggcggcattggcatagacgatgccgccctcggcgtcgaggatgatcaccgccgaggccagcaggtcgagaccttcgagggaggggaacggagtcttcatggcggtagtcggggagcctggattcatgtcgtggagcagagtcgttcgtgcgggaacactagcaagtttcgggcgcgctcccatgaggatgcaaggaatgcacacaactcaggggacttgccggcgcgcataaaaaaaggggaagcaagcttcccctttttgtccggatgcggattcgattacagcgagtagtacatgtcgaattcgatcgggtgcgtggtcatgcggaagcgctggacttcctgcatcttcagatcgatgtaggcatcgatcatgctgtcggtgaacacgccgccacgggtcaggaactcgcggtccttgtccaggtgttccagcgcttcgtccagcgaggagcacacggtcgggatcagcttgtcttcttccggcggcaggtggtacaggtccttggtcgcagcttcgcccgggtggatcttgttctgcacgccatccagaccggccatcagcagggccgagaagcacaggtaggggttggccaggggatccgggaaacgggtctcgatacgacggcccttggggttggccacgtgcgggatacggatcgaggccgaacggttgcgggccgagtaggccagcttgaccggggcttcgaagccggggaccagacgcttgtaggagttggtgccggggttggtgatggcgttcagagccttggcgtgcttgatgatgccgccgatgtagtacagcgcgaactcggacaggccagcatagccgtcgccagcgaacaggttcttgccatccttccagacggactggtgaacgtgcatgccg

SEQ ID NO: 7 - DNA, 5524 bp, synthetic construct
  misc_feature 1..5524: note = glnA upstream homology arm - lac promoter - lacI CDS - tac promoter - glnA downstream homology arm sequence cassette
  source 1..5524: mol_type = other DNA
cccgcgccgcaagcgcaacagcccgccttccagcgattcgctctcggaaatgcccagcctctcgccacgcgagcgcgaagtgctgcgctgctgcctggatggcctctccgtcaccgccatcgccgacaagttcgcccgcagcgtcaagaccatcagcggccagaagcaatcggccttccgcaagctgggcgtgcgcagcgacaacgagctgttcaagatcgaataccagatcaaggacctgtaagaccagcaaggagacagaagatgaaccgcagtgagcctagggtcttgctcgtcgatgattcggcgacgatccgttatgcgatgcagaaccgtttgcaatcgctgggctgccaggtccaggtggcggccgacggcgccgccgcgctggcggcgatccgcgcccagccgcaggggctggtgctgctggattgctttctgccggacctgcccggccacgaggtggcgcgtcaggtgcgcgccctggagcaagaggacccgcagcgtccctacacgcccttgatcggcatttccgccgaggctgatgcggcgcacgtacggctatgcctggacggcggcatggatggcatgctcgacaagcccgtgcagaccgccgacctgcgcaagatgctctcgctctggtgcgatcatgaatgtggcgacgagggcgccgcgctgcccccgcgcgaggaatatgcacgcgacctggcgcgcttgttcctgagcacctcgcagcacgacctggccgccctgcaacaggcccaccgggccggcgaccaggccaccatgggcaagctggcgcaccgcatgaagggcgcggcgctgaccatgcagcgcagcgagatcgtggccctgctggagtgcctggaagaggccatgcaggacgactacgacacccaggacgccatctccctcttgttgagttcactggagaaggtattggccgcaagcgcggcgccgggctaggagcgatcccggccgacgcacagcccggcgctcaggcccccacccacggcaggcccgccttgcaccagccctcgacggtcttgcggtggcccgcggcgtccttgttgccctcgaagccttcgaggatgtcgaagcagtgggtatagccatgctcggtcgccagcttggccgcatgcttggaacgcacgccggaacggcacaggaacagcagcacatcgtcagggttggtgacttcccgcagctgttccaggaattgcggattgggcttgccctcggggtagagattccattgcaccgctagatgctgcgccggcgcgatctggaccacgccgacccagtcgcgctcggcgcgggtgcgtacatcgaccagcttgatgcggctgtcgccttcgagcagggccagtgcttcggccggcgtgactgcgccgcgatagggaagctgtgcatcgacggcgcgttgggtggcggcggagaggatatcttgagtgctcatgctgggttcgctatcaagtgtcaggtataagtgtcaggtatcgttgatcgtggatcgccgtgttgcgccggggcaagccccccaggatatcgcgacaggcaatcgcaccgggagctcagattctaaatccaaaccggcgcacgtccccggcctcgcttgcttcggttgcttcggttgcatgatgtggccgctttggtgcgtacctgctgcggcagcaagcgctgccccagaaaagcgcaggcaccaaaacagggcaattcagcttgtttcttccattagtgcattttaatggtgcatttagataaaaacgcactaaaataatgcatcccctggcggcctctgcaccgcagggatcggcccgcaggccgcgtcctccctaaaatatctcatcctgtgcgcaccctaaaggttggtggcatggaatctgctatcatccgccactgagttcggcgcaagaccgttgtatcaaatgcaaagatccagtcgcagaaaacggctcaacaccccaatttatactacttcctcgttttaggagattcgccatcaaataaaacgaaaggctcagtcgaaagactgggcctttcgttttatctgttgttttggtgatgtcggcgatataggcgccagcaaccgcacctgtggcgccggtgatgccggccacgatgcgtccggcgtagaggatctgctcatgtttgacagcttatcatcgatgcataatgtgcctgtcaaatggacgaagcagggattctgcaaaccctatgctactccctcgagccgtcaattgtctgattcgttaccaattaggatccttatcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagtaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccaccatcgccgcttccactttttcccgcgttttcgcagaaacgtggctggcctggttcaccacgcgggaaacggtcatataagagacaccggcatactctgcgacatcgtataacgttactggtttcatattcaccaccctgaattgactctcttccgggcgctatcatgccataccgcgaaaggttttgcgccagcactagaacttgcggcggatactgtcccatgttgacaattaatcatcggctcgtataatgtgtggaattgtgagcgctcacaattaaagaggagaaattacatatggcaaggacggccgcagaggttttgaagatggtgaaggacaacgaagtcaagtttgtcgacttccgtttcgctgacacccgtggcaaggaacagcacgtttccgtgcctgtttcgcatttcgacatcgacaagttcgaatccggtcatgccttcgacggttcgtcgatcgccggctggaagggtatcgaagcctccgacatgctgctgatcccggacccgaacaccgccaacatcgacccgttcatggaagagacgaccctcttcatgcaatgtgacgtgatcgagccgtccgacggcaagggctatgaccgcgatccgcgctccatcgccaagcgcgctgaagcctacctgaagtcctccggcctgggcgacaccgcctacttcggtccggaacccgaattcttcatcttcgacggtgtgcgttggggcgccgacatgtccggttccttcgtcaagatcgactccgaagaagcttcctggagcaccggcgccaagatcgaaggcggcaactccggccaccgtccgaccgtcaagggcggctacttcccggttcccccggtcgacagcttccaggacatgcgttcggaaatgtccctgatcattgaatccctgggcatccccgtcgaagtgcaccaccacgaagtggctggcgctggccagaacgaactgggcaccaagttctcgaccctggtcgagcgcgctgactggacccagaccatgaagtacgtgatctggaacgtcgcccacacctatggcaagaccgcgaccttcatgcccaagcccctggttggcgacaacggctccggcatgcacgttcaccagtccgtctggaaggatggcaagaacctgttcgctggcgacggctatgctggcctgtccgagttcgcgctgtactacatcggcggcatcatcaagcacgccaaggctctgaacgccatcaccaaccccggcaccaactcctacaagcgtctggtccccggcttcgaagccccggtcaagctggcctactcggcccgcaaccgttcggcctcgatccgtatcccgcacgtggccaaccccaagggccgtcgtatcgagacgcgtttcccggatcccctggccaacccctacctgtgcttctcggccctgctgatggccggtctggatggcgtgcagaacaagatccacccgggcgaagctgcgaccaaggacctgtaccacctcccgccggaagaagacaagctgatcccgaccgtgtgctcctcgctggacgaagcgctggaacacctggacaaggaccgcgagttcctgacccgtggcggcgtgttcaccgacagcatgatcgatgcctacatcgatctgaagatgcaggaagtccagcgcttccgcatgaccacgcacccgatcgaattcgacatgtactactcgctgtaatcgaatccgcatccggacaaaaaggggaagcttgcttcccctttttttatgcgcgccggcaagtcccctgagttgtgtgcattccttgcatcctcatgggagcgcgcccgaaacttgctagtgttcccgcacgaacgactctgctccacgacatgaatccaggctccccgactaccgccatgaagactccgttcccctccctcgaaggactcgacctgctggcctcggcggtgatcatcctcgacgccgagggcggcatcgtctatgccaatgccgccgccgagaacctgctggaaagctccttcaaggtgctctcgcagcagaagctctcggagctcttcctgaacggcaaggagctctcggccctgttctaccaggccatcgaacaccagttcgccgacaagcgcctggacctggtgctggaacgcgccaaccgcgaaccgctgcaggtccacaccatcgtcacgccgctggacaaccccgacatgccggtgctgctggagctgcgcgaaaacgtccagcaattgaagctggaccgcgaagagcgcatcttcgaccagagccaggccaacaaggaactgatc

SEQ ID NO: 8 - DNA, 3999 bp, synthetic construct
  misc_feature 1..3999: note = nifH upstream homology arm - nifH downstream homology arm sequence cassette
  source 1..3999: mol_type = other DNA
ccgtgcacgaacggcagcgtgttctggaagccgacggcggcatacaccgcccccagcggctggcaagccttggccggattgatggtcagcgcctcgcgggcgaagttcttttgctggtattccttggtcttggtccattcggccacgtcctgcaccttgtccagcggaacggcaaactcgaactcttctttcttgcgggcgaacagttcgcggtattccggctggcggaacagattctcgtggtccagaacgctatcggcgttttgcggcatgatgataccccttcaattcaactgatcaaatgatccaggctcgggtcggatggacgatgagcgatcgacacagtcctgcgcccatccatcccgaatcgattcagctcaggccttccacggagccttggccatgccccacaccgggctgttgatggccatgtccatgtcgcgggcgaagatggcgaagccgtcatagccgtgatacgggcccgagtaatcccaggagtgcatctggcggaacggcacgcccatcttctggaagacgtatttctccttgatcccggagccaaccagatcgggctcgatcttctcgacgaacttctcgaattcatagctggtcacgtcgtcgtagatcagcgtgccgtcttccacgtagtgggtggtgcgctggtagtcgtcgttgtggccgaattcatatccggtaccgaccaccttcatgcccaggtcaccataggcatcgatcacgtggcgcgggcgcaggccaccgacgaacagcatcacggtcttgccttccaggcgcgggcggtacttggcgatcacggcatcggtgagggccttgtacttggcgatgacacgctcggcgccttccttgatcttgtcgtcgaagtggctggcaatctggcgcagcgacgcctcgatctggctggggccgaagaagttgtactcgacccagggaatgccgaacttctcttccatgtggcgcgagatgtagttcatcgagcgatagcagtgcagcacgttgagcttggccttgggggtgttttccatttcggcgagggtgccgtcgccggaccactgggcgatcacgcgcaggccgatttcttccagcaggatgcgcgaggaccaggcgtcgccgccgatgttgtagtcgccgatgatggccacgtcatacggagtcgccacaaactggttcttgtccgggtccatcttgtccagcacccattccttgatggcgtcgttggccacgtggtggcccagcgactgcgacacgccacggaagccttcgcagcgcaccggcacgatggtatggccctcgtattgcttggactttttcttggagaccgcttcgatgtcgtcaccgatgagaccgatcgggcattcggattgcaccgaaatacccttgttgagcgggaacagttcctggatctcgtcgacgatcttttccagcttcttatcgccgccgaagacgatgtccttttcctggaaatcggacgtgaactgcatggtcacgaaactgtcgatgccggtcttgccaatgtaatagttgcggcgcgagccccaggagtactggccacagccgaccggaccatggctgatgtggatcatgtccttgatcggcccccagaccacgcccttggaaccggcataggcgcagccgcgaatggtcatcacgccggggatcgacttgatgttggatttgacgttgcaatccgacttgccctcttcctgggtggtcaggtgcttggcgcgacgcttggcggtcttgtccggataggccttgaggacctcattgatcaattccgtattgcgtgccgtggtttcttcaactgtgaggctcatgggccctccgataaaaattagtcgcgggggacatacccggtcgtatgccagtccggtaaacaaacctgcgctaccctttttcttgcggtcccggctgccttcgcttctgaacacagaggcgcgagcggccaggaccttgcagcatcaacagcaggcagacgacgatgcttgattctcctagaaataagcggtgtagctctggtgtaagaaattaaggtttcgttcaaagcagttttgtcatcacacttgcagccgttggggcgccgcttcgaaacttttattggtcttcgtctcaagtcacgcattcggcaagtcatcgtcggtgcacacctctctttgcaaatgccgtgccaccatcaaaaaacgattccaagccattgatttaattggtgaaaacagcatttcgcgcagcctctcgacccacttctttctggtgttacaaaccgcacaaacggcacctggtctgtagggaacgatacaagcccgcatccccgacacaaccgtgtacccaaccaccctgttgcacccctctcggcccgctggcccgcagccacgggccttgtcacgccctgctgaagacaagcagaggcggctcgctgataacgggaacgatgtttgctatcgggatgaatggcagggcaaagaaggctgcgccaagcggcccggtcagtgatccactcacgcaaggagcaaaccatgcaaatcggtgtagacagccagcgcgcggtcgacaacaaacgggtcttcatcgtcgactgtgatgaagtcagcggcttggcgctgcagttcatgctcgccgacgagtgcgaagcgcatctgctggcggatgtggacaatgccttgcgcaagggccgggattggccgcccgacctgctcttgctgggccagggcaatctcgtcaaggagggcatggaggtgctggcccaattgcgccagcaacatccggccttgaagatcgtagtggtgtgcgacagcgccaatgaccgccatgttcgccaagccctggcgcaaggggccaactccacgctgctgcgcccgcttacgctggagagcgtgcgccgcaaggtcgatgcccaactgggccgacgcacgcccttgggcctgggcatcgcggtgacggtgatgtaagaccatgtgcgcgactcgcatccccttgaagctgccgcatcgggctaccccgatgccgggcgcatcgaagtctgcgccgctggccaatccgcattaccagcagatcggcggcgagccggtcgtgcggcaactggtcgaccgtttctacgccctgatggaccagttgccccaggcggccgtggtacgcgccatgcatccgggcgacctgaccccggccaagcagcgcctgttcatgttcctctccggttggctgggcggcccgccgctgtatgccgaggccttcggccatccgcgcctgcgtcacgcacacgcgggctttgccgtcgatgctgccgcacgtgatgcctggatggcctgcatgaaccaggcactacaagaactggtacccgacgaggtgttgcggcagcaactgggtgccgcatttttcaagactgccgattttctgtgcaaccaataaggaaacttcatgagcactctcgcaaatacccccctggccgatctcgacaatgatggccgtctgctgagcccggtcttcaagggcccgaccaaacgcaatggacgcgtcgccttccgtggcgaaatcgcgctcaaataccaggccagcctggccgatgaaaagcgccccccggaaatcaagctcgaccaggtgatgacggtggccgacgtgggcgccaccaccattcctttcctggccggcatggcgccttcgctggaacatatcaagctgctgtgcgaagtgctgggggacaagctggctgccgatggcaaatatttcttctttgcctgcaacctggacatttcgctgcggttccagattccttatggcggcgccaccttctacgtgatgccgctggacgaatcgacggtctataacgagctgctggaattgttccgcatcgagcgtggcgatctcaagaaactcgacaccgccgaaaagattctggcggtggccgacggtgccctgcgcttcaagggcaagttcgaggagatcagcttccagcgcgggctggaggtgatggggccgatcaagatcaaggaa

SEQ ID NO: 9 - DNA, 4000 bp, synthetic construct
  misc_feature 1..4000: note = nifA upstream homology arm - nifA downstream homology arm sequence cassette
  source 1..4000: mol_type = other DNA
tttttctccttgcgtgaaactgattggctgcttacgctgcggcggacgtcgactgcaccgaggtatacgccccctggcggatatcggcgtcgcctcgtggccgatgctcgatctgtccgctgcgcacgcgttccaggtaatccatgaagtaggcgatgacggattgctcgatgaattcatgggcataccggtccaccggctcgatgcccaccttctgcagatcatccttgggacagccgccgatcttggccaccagcactgcatggcagtcgttgatggcgcggatgacggtctccagtgcatcctcctcgccatagccgccctgacagtactggtccacgcgccggtgtccgacgaacttgctgccggcgctgctgacttcatagatctggaattcgctgacgtgtccgaagtgctcgttgacgcgtccctgccccttggtcgccaccgccaccagcaaggacaggccgctctggtctgccatctgggccagctcctgctgctcggcggccttggcggccgacttggcctggcgttcctgttccaccagctcctggtaagccttgcgggtggcgccgtcataggccacttccatggcctcgatcttttcggtggtgaattcggcgctgcggtcttcacccagcaggcccaccgcatcggcgcggcactggcggcagtggcgcatcatgttcatctcgccttcacaggcatcctgcaaggccttgagctcctgcgcgctggggccgcgctggccgttcaggccaaacacggtgccatgctcgggcgctgaaatgagcggcatgatgttgtgcaggaaggcaccgcgtgacttgacggcgcgattgacttccaccagatgctggtcattgatgcccgggatcatcaccgagttgaccttgcacaggatgccgcgcgccgtgagcatctccagtcccagcatctgccgttcgtgcaggatgcgggccgcgtcgataccggtccagcgcttgttctggtaatagatccagggatagatgtgctgtccgatctcgggatcgaccatgttgatggtgatggtgacgtgatcgacgttaaaggcagcaatggtatcgatgtggtcaggcaaggccaggccattggtcgacaggcacagcttgatatcgggcgcggtctgcgaaatcagctcgaaggtcttgaaggtcttggccggattggccagcgggtcgccaggaccggcaatgcccagcaccgtcatctgcggaatggtcgaggccaccgcaaagaccttcttggccgcttgttcgggcgtgagcttttcgctgaccacgcctggacgcgactcgttggcgcaatcgtatttgcggttgcagtagttgcactggatgttacaggccggtgccaccgccacgtgcatacgcgcatagtggtgatgagcctcttcgctgtagcagggatggttcttgaccttttcccaaacctcgggcgccatgtcggcgggaccatcggatgaaccgcaactggctttgccgccttcactgctggtgccacagcctttgtgttcggctaccttgtccagcaaggtgcctagcgatttgatgtcctggataccgacgtattgtgtgggctgcatggatagtccctttcaggaaagtgatgcctgggccttctcctgaggacatagccaaaaccgtgccagacaaaataatcaatcaattcaatgacatagcggagaattgcgtcgtacggcgatgtgaggatagcgacaatcggcgatacattgtgggtggcgcttgtcatgaacacgacaatgcagcacgggccggcgcctgcaattgagcgcgccgcaatgagcctgacgcagtggagaaggagaaggagaatgcgaggacgtgccagccggggaatccatcaatacaaccggctggcgcgacgccgaagagaccgcgcgcgcagggcgaggacggcaatgagaaaaagactttggatctggcagcgtattacgtcacgctcgcagatgagttctgactctcctaacgtgaatactggaaccccatgcgctgcctgagagcgctagaacccgccttgctactctgtcgtgagatccgagatcctgaaaaacggcagaaaaagcgccgtgtaattaccgaatagttaccttgctggtcaagcaaacttcatgcccaagcagatgcatgaatcgatctcgccaagcaaccccacacaatgccggctttcctcgatgtcgccttggcgggttggcgacaacggtgtcgtctttgtcggatttacgacaatcatcgccactggcgcagcaatcagaaatcgtcgctatcccattgatttaaaagaaaaatattgtatggcatggttttggctattcatacctgcatcggccgctcaccggccaagcgaaacgacaaagcagagcaagccggtagcccagccagcgttatgtagcgagatatacagacctccaacatccaccatcgcccactatgaatgtgccttcgacttccgaacccggtgcaccacgtgcgtgctgggcgccctgctcccaggcatgttatctgaccctgaactggccggtcgccggcccggcgatggcgcgcggcacgcaacaggtgatatgtgagccgccacaattacctcaaccggcgcaccggttgctggcgctgtgtgatgaatccggcttgcgcaaactggtgctgcatcatatgcggcgcctgcgccattccccgctgttcgcacgttcgcacaattgttttgattgcgtcagctcgcgcatcgccgacttcgtggtggagtcatgcggcggtccgctctactacagccaacgccatgcccatctgcaagcgggcgcaggcttgccgttgctattggacgaagctgggcgcgaactgtggctggtacagctttggcatacctttgacgatatcggtttcccgcccgcgctgcgggccgacttctgggcctgggccgaacccttgtcgattcatctgctggtacgtcatgcgcgcgtcgaaccgccgcggcgctacccctatgagctggtacgcagctggttccactcgcccgccaccgacatgctgccgccgatagcggatctgatccggccatcgggaagatccgaatcatgacctgcgccatccggcatcgcatccccgcatctgtttcttgatgcggccggtctgagcgatgtccaactctttttcccgtgaacctgccatgccggattacccgcaacgctacggcgtcttcctcaatgtcgaaaatccccatggcaacgcacgcgatgcgctgttgcagaccgtcgatcatgccgtcgtggcagaggccctgggctatcacgatgtctgggtcgccgagcaccattacagcagcttcgccatcggcagcgccttgatggtgctgctggcccagatcgccgcacgcacttcgcgcatccgcctcggtacgggcgcttcgttgctggcgctcaatgatcccttgcgggttgccgaggacgtcgccacgctggacctgctcagcaacggccggatggaattcggcgtggcacgcggcggcccgtttcccgagcaatatcgtcacgccggattgagcggtgccgatcaggcgcgcgcacgcatgattgaagcactggacgtcatccagcgcgcatggcaaggcgtgccattcagtaccgagggacgattttttcactacgacagagtgcagttgcaaccgcagccgctgcaacgtcccatcccggtatggctggccagcatgagcgaagattcgcttgatctggccgcacgccaggattacggattgatggccacaccctccgccgatcttgagcaggtgctcacgcgggtggctgcacagcgtgcgcagcgcggtcacttcccgtttgccatcgcccgctttttccattgcgaacccgaggcgcacaaggcgcttgcccatggtctggcaggtgtgcgtgcgtatccgcgcatgatgcaagtgcaattcgcgcccggtggcaccccgcccatgttcc

SEQ ID NO: 10 - protein, 11 aa, synthetic construct
  REGION 1..11: note = degradation tag amino acid sequence
AANDENYALAA

SEQ ID NO: 11 - DNA, 8885 bp, synthetic construct
  misc_feature 1..8885: note = glnK upstream homology arm - glnK downstream homology arm in vector including gentamicin selectable marker and human herpes virus 1 thymidine kinase counter-selectable marker
  source 1..8885: mol_type = other DNA
ggaccgaaacaaaaaaagacgcttttcagcgtcttcaataattggtttggtaccgagttatagtccagggcgttactttagttggcttctcccatctcgcgtgcaaacgtgcgcgctaaatcacagatggtcggaatcgagcccggagtggtgacatgggtctgcaccatgccagaggtcagttgtagcagcgcatctctgcaaccagctggcgactgatcgtaatccaggataaatacgtgcattgagcgcagtcttttagccagaacgtccagggcccaggcgaacacgttatacaagtctccattcggggcgagaagttcgggtgcacgaaacaacgtgaacagtgtatcccctatatgagggcgagggcctgcgttggactgaggttccgcaccttgaggagggacggccgtacctgagagctggccccaatcctctcgccagctaccaccacattgaagataacgaactgtattcgccagtaagccatacacgcgacggatcgcagctagcatggccaagtctaacctctcccccggacgctggcgtttagccagacgatcgatatgtcgatcttctggcaatgcgcccagcacaatgttggtcccggggagcgtagggggaataagcgcaacaaatgccagaacagcttgcggagtcatactacccatcaagtaacgcgcggcaggataacacagtaacgcggcaattgggtgacggtcgaagatgagtgtcagtgccggaggtggagcatggctggagcccgcttctcccccaatgtgcggggctagtacagcatcagtgacagcgtacggcatacccatggtgatctgcgccgaggtcatgaccactgcggcatcgccagcagagatttcaccctgatccagtcgatgttgcgtagtataaatattcgctatcgtttcgctggcgcccagaactctccagtaggtcattggttccggcacatacacgatgtcatcgcgggaacccagcgctaccaggagctgggtcgtggtagttttgcccatcccgtgcggtccatcaatataaacccgaagtaaggttggcatcttctgttcggggcggacctccgttgcttcttgctggcgtcgcggacgcaaggctgtacggcggttagaatggcccctactccgcgcagcttggtcaaaggctgaggcatgctgatgaccagggtatgaagccattatttcctccttacgtctaggaacgctagcataatacctaggactgagctagctgtaaaaagatcccctgattccctttgtcaacagcaatggatcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcggccaattcgatctagaattccattgagtaagtttttaagcacatcagcttcaaaagcgctctctcgtcggcattggcggcgggcaggatgaaggcgcgccgccgttcgtcggcgctggcgccgtgctgcatggcgcaggtcatcgccagggcgccgcggatgggcaacaactggccggagagggatagctcgccggcgtattcgtagtcgtccagccggtcgccggggatctgctgggaggcggccaggatgcccagggcgatggccaggtcgaagcggccggattccttggggaggtcggcaggcgcgaggttgacggtgatgcgccgcgccgggaattcaaagcgggcgttctgcaatgccgcccggacgcggtcacgggcttccttcacctcggcctcgggcaggccgacgatggcgaaggagggcaggccattggccagatgcacctccaccgtgaccggtggggcgttcatgccggccagggcgcggctgcgcaggaccgccaggctcatcgcgatgggcctggctgcctgttacctatatagagaagagagtgtgatcgaagtgaaagtaggtgcgccaggatcatggagtctgtttttgctgggcttccagctcggccacacgggcctccagggcttccaggcgggcgcgggtcttggccagcacctgagcctggatgtcgaattcctcgcgcgtgaccaggtcgagcttggcaaagccctggctgagcatggccttgacgttcttttcgatgtccttggccggggagttttcgattgccttgttgatcttggattgcaggtcttcaaaaaacgggggcttgtccatgtcggatctccttggcagataagctgtagctggaatgcaagtttagcggtttcctgcccatcctcaaaccgccttgcctggatgagaccgcatttttaccgccaagtgcggcctgtgaagttttttccttcaggaaatgcgccgtcttggtgatccggaacgcccctgctggccgcgcaccaaaatgtgattgccggaatggaagcagggttaacaaaaaacaagcaatatcgcaccaatcacgtgcattttcccttggtctgcactattttgaactgaagttagaaaaaaaacgtgaccaatctccggtttcacctaaaaacacaagcctgccttgccgaaagaacatggcacggctcctgctacagggtttgagcaaacacaaaaaacgacttttattgttcaaacacaaaccagagggaaagtcatgaagaaactgattcttgcaacagccatcgccgcatccttcgtttctaccgtggcccgtgcagaagacgccaaaccagataatgagttcagcttcaacgccgctgtcgtgagcgactaccgctaccgcggcctgacccagacccgcttcgacccggccctgcaagtcggtgctgactacacccacaaccccaccggcctgtacgccggcacctggctgaccaacatcaagtggatcaaggacgctggcgccgtggccagcccgccggtcaacaccaagggcgacatcgagtgggacatctatggcggcaagcgcggcgacatcggcggcggcttcacctatgacgtcggcggcctgtactactactacccgggcaacaccctggccgacagcggcgccaagaacgccaacaccttcgaactgtacggccaggtcggctacggtccgggctacttcaagtattcgcaatcgctgaccaacctgttcggcgcagccaacagcaagtacagccagtacttcgacctgggcgccaacatcgagacctactacggcgtcatcctgggcctgcacgtgggtcaccagaacgtgcgcggcaaccccaaccagttcagctacaccgactggaagatcggcgtgtccaagaccttcgacgatctggccggcatcaccctgggcctggcctatgtcggcaccgacctcaaggatggcgtgaccaccacgccggcttcggatggcctgaagaacgtcggcaagaacggcttcgttttctccctcagcaaaactttctaacaagaggttcgtaaaaggggaactctaatgagcatgaagaaaatgttttccggcgcgctcgccattggcgcgctgctgtttgcagtcagttttactgctccggcccaggcgcaggatgcgcccaagcctgaagctgcagcggctgccgcagcggccccggctgctgctcccgctgcacccaaggcggaagctgccgctcctgcggccgctgccccggccgcaccggctgccgctgccgccgctcccgcaccggtccccaacaagggcgataccgcctggatgctggtctgcaccgccttcgtcatcctcatgaccttgccgggcctgggcctgttctacggcggcctggtgcgcagcaagaacatgctgtcggtgctgatgcagtgcttcatgatcttctcgctgatctacatcctgtggttcgtctacggctacagcatcgcgttcaccgagggcaatgccttcttcggtggcttcgatcgcctgttcctgcacggtctgacgggtgactccaacaccggcaccttcagcaagggcgtggtcattccggagctggtcttcgtcgccttccagggtgcgttcgcggccatcactgtggcgctgatcattggcgccttcgccgaacgcgtgaagttctcggccgtgatcctgttctccatcctgtggttcaccttctcctacctgccgatggcccacatggtctggttctggccgggtccggacgccttcaccgacgctgccgctggtgaaaaggccaccgccatctcgggctggctgttccagaagggcgcgctggactacgcaggcggcacggtggtgcacatcaacgccgctatcgccggcctggtcggttcctatgtgatcggcaagcgtatcggtttcggcaaggaagccttcaagccgcatagcctgaccctgaccatggtcggcgcctcgctgctgtggttcggctggttcggcttcaacgccggctcgggtctggaagccaacggcactgctgccctggccttcgtcaacaccatcctggcaacggcggctgctgtgctgtcctggagcttcggcgaatggatcggcaagggcaagccctcgatgctgggcgccgcttccggcgcggtggctggtctggtggcgatcaccccggcggctggtttcgtgggcgtcatcggcgccgtggtcatgggcctgatcgctggcctgctgtgcctgtggggcgtgagcggcctgaagaagatcctgggttccgacgatgcgctggacgtgttcggcgtgcacggcgtgggcggcatcctgggcgccatcctgaccggcgtgttcgcagcccccagcctgggcggcaccggcatctacgactacgtggccaacgctgttgcgcctgactactccatcggcggccaggtctggatccagttccagggcgtgctgaccaccgtggtcctgtcgggtgtggtctccttcatcgcctacaagatcgttgacgtcgtcatcggcctgcgtgtgaccgaggaagaagaacgcgaaggcctggatatctccagccacggcgaatcggcttaccactaagcctctgccgtactgatcgactgagtctgcagtattccttcggggccggcgacggcctctttcaaaggcgccttcgggcgccttttttcattgcctggatgcaagctgcttttttctgtggcattcgcgcgtcgccccggataattcccgactatttgagtcggactctttaaaatccctccatcatcgccatctgaacactcccagcgacgctttcgcagcgggcatcgccagggccggcctttgccggaatggcaacagtggcttgtgtcgcgtcgcaacacgatgtcaggaatccgctaaaatcgatcctcgctgccggcatttacatatcgttaaggtctcttatggtcccgcatctcgtcaccgctctgaatggtcctctgctggagctggagaagaaaatcctgggcgccacgccggccatcgaacgctggttccgcatggaatggcctccttacgcagggcatccatttattactcaaccgtaaccgattttgccaggttacgcggctggtcctctagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcgtcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgactcgagcgtcgatttttgtgatgctcgtcaggggggcggagcctatggaaaaacgccagcaacgcggcctttttacggttcctggccttttgctggccttttgctcacatgttctttcctgcgttatcaggggattccttaaggtatactttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccgataagctctgataccgctcgccgcagccgaacgaccgagcgcagcgggtcagtgagcgaggaagcggaagagcgcccaatacgcaaaccgcctctccccgcgcgttggccgattcattaatgcagcgcccaaacataacaggaagaaaaatgccccg

SEQ ID NO: 12 - DNA, 10804 bp, synthetic construct
  misc_feature 1..10804: note = amtB upstream homology arm - P65 promoter - Hs NifA CDS - amtB downstream homology arm in vector including gentamicin selectable marker and human herpes virus 1 thymidine kinase counter-selectable marker
  source 1..10804: mol_type = other DNA
ggaccgaaacaaaaaaagacgcttttcagcgtcttcaataattggtttggtaccgagttatagtccagggcgttactttagttggcttctcccatctcgcgtgcaaacgtgcgcgctaaatcacagatggtcggaatcgagcccggagtggtgacatgggtctgcaccatgccagaggtcagttgtagcagcgcatctctgcaaccagctggcgactgatcgtaatccaggataaatacgtgcattgagcgcagtcttttagccagaacgtccagggcccaggcgaacacgttatacaagtctccattcggggcgagaagttcgggtgcacgaaacaacgtgaacagtgtatcccctatatgagggcgagggcctgcgttggactgaggttccgcaccttgaggagggacggccgtacctgagagctggccccaatcctctcgccagctaccaccacattgaagataacgaactgtattcgccagtaagccatacacgcgacggatcgcagctagcatggccaagtctaacctctcccccggacgctggcgtttagccagacgatcgatatgtcgatcttctggcaatgcgcccagcacaatgttggtcccggggagcgtagggggaataagcgcaacaaatgccagaacagcttgcggagtcatactacccatcaagtaacgcgcggcaggataacacagtaacgcggcaattgggtgacggtcgaagatgagtgtcagtgccggaggtggagcatggctggagcccgcttctcccccaatgtgcggggctagtacagcatcagtgacagcgtacggcatacccatggtgatctgcgccgaggtcatgaccactgcggcatcgccagcagagatttcaccctgatccagtcgatgttgcgtagtataaatattcgctatcgtttcgctggcgcccagaactctccagtaggtcattggttccggcacatacacgatgtcatcgcgggaacccagcgctaccaggagctgggtcgtggtagttttgcccatcccgtgcggtccatcaatataaacccgaagtaaggttggcatcttctgttcggggcggacctccgttgcttcttgctggcgtcgcggacgcaaggctgtacggcggttagaatggcccctactccgcgcagcttggtcaaaggctgaggcatgctgatgaccagggtatgaagccattatttcctccttacgtctaggaacgctagcataatacctaggactgagctagctgtaaaaagatcccctgattccctttgtcaacagcaatggatcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcggccaattcgatctagaattccattgagtaagtttttaagcacatcagcttcaaaagcgctctgggcgaaggagggcaggccattggccagatgcacctccaccgtgaccggtggggcgttcatgccggccagggcgcggctgcgcaggaccgccaggctcatcgcgatgggcctggctgcctgttacctatatagagaagagagtgtgatcgaagtgaaagtaggtgcgccaggatcatggagtctgtttttgctgggcttccagctcggccacacgggcctccagggcttccaggcgggcgcgggtcttggccagcacctgagcctggatgtcgaattcctcgcgcgtgaccaggtcgagcttggcaaagccctggctgagcatggccttgacgttcttttcgatgtccttggccggggagttttcgattgccttgttgatcttggattgcaggtcttcaaaaaacgggggcttgtccatgtcggatctccttggcagataagctgtagctggaatgcaagtttagcggtttcctgcccatcctcaaaccgccttgcctggatgagaccgcatttttaccgccaagtgcggcctgtgaagttttttccttcaggaaatgcgccgtcttggtgatccggaacgcccctgctggccgcgcaccaaaatgtgattgccggaatggaagcagggttaacaaaaaacaagcaatatcgcaccaatcacgtgcattttcccttggtctgcactattttgaactgaagttagaaaaaaaacgtgaccaatctccggtttcacctaaaaacacaagcctgccttgccgaaagaacatggcacggctcctgctacagggtttgagcaaacacaaaaaacgacttttattgttcaaacacaaaccagagggaaagtcatgaagaaactgattcttgcaacagccatcgccgcatccttcgtttctaccgtggcccgtgcagaagacgccaaaccagataatgagttcagcttcaacgccgctgtcgtgagcgactaccgctaccgcggcctgacccagacccgcttcgacccggccctgcaagtcggtgctgactacacccacaaccccaccggcctgtacgccggcacctggctgaccaacatcaagtggatcaaggacgctggcgccgtggccagcccgccggtcaacaccaagggcgacatcgagtgggacatctatggcggcaagcgcggcgacatcggcggcggcttcacctatgacgtcggcggcctgtactactactacccgggcaacaccctggccgacagcggcgccaagaacgccaacaccttcgaactgtacggccaggtcggctacggtccgggctacttcaagtattcgcaatcgctgaccaacctgttcggcgcagccaacagcaagtacagccagtacttcgacctgggcgccaacatcgagacctactacggcgtcatcctgggcctgcacgtgggtcaccagaacgtgcgcggcaaccccaaccagttcagctacaccgactggaagatcggcgtgtccaagaccttcgacgatctggccggcatcaccctgggcctggcctatgtcggcaccgacctcaaggatggcgtgaccaccacgccggcttcggatggcctgaagaacgtcggcaagaacggcttcgttttctccctcagcaaaactttctaacaagaggttcgtatgaaactgatcacagcgatcatcaaaccgttcaagctggacgaggtgcgtgaagccctctccgcgatcggggtgcaaggcatcaccgtgactgaagtcaagggcttcggtcgtcagaagggccataccgagctgtaccgcggcgcggaatacgtggtcgatttcctgcccaagaccaagatcgaggcggcggtggacgacgccatcgtcgaacgcgcgctcgaagccatcgagaccgctgcccgcaccggcaagatcggcgacggcaagatcttcgtccaggacctgctggacgtgatccgtatccgcaccggcgagaccggcaaagacgctctgtaaaaaaggggaactctaggccgccgtagtgtccctgcccggctcgtgggacggcgcgcatcccggtcagccgggatgtttcacgtgaaacatccgttcgcatcctttctccaaaaaaccctcttgacttggagtccgctcgaagccctatgatgataggcgaacccatcgagaggagcatctatggccactattctcgacgaccgcagtgtgaatttagaactcgtaacgatttacgagatcagcaagatattgggttcatcgctggatctatccaagaccttgcgcgaagtcctcaacgtcctctccgcccacctggaaacgaaacgggtattgctgagtctgatgcaggactccggtgaattgcaactggtcagcgccatcggcctgagttacgaagaattccagagtggcagataccgggtcggcgagggcatcaccggcaagatcttccagacggaaactcccatcgtggtgcgcgacttggcccaggagccgctgttcctggctcgcaccagcccgcgccagtcgcaagacggggaagtcatttcctttgtgggcgtgccgatcaaggctgcccgtgaaatgctgggtgtgttgtgcgtttttcgggatggacaatcgccctcgcgtagtgtcgaccatgaggtgcgcctgctgaccatggtggccaacctgatcggacaaaccgtccggctgtatcgcagcgtagcggccgagcgccagcagctgcaagaggaaaagcgccagctgtcccggcaactccagggcaagtacaagctcgataacgtaatcggcatttccaaggcgatgcaggaagtgttcgcgcaggtacaccagtccgccccctcgcgctcgaccatgctgttgcgcggcgagagcggcaccggcaaggaagtcatcgcgcgcgccatccattacctcagtccgcgcaaggatgggccattcatcaaggtcaactgtgcggccttgtcggaaaccttgctggaatccgaattgtttggccacgagaagggcgccttcaccggggcccagggtgaacgcaaggggcgtttcgaactcgcccatggcggcaccttgttcctcgacgagatcggcgagatctcgccggccttccaggccaagctgctgcgggtattgcaggagcgtgaattcgagcgggtcggtgggtcgcgttcgatcaaggtggacgtgcgtctggtcaccgccaccaaccgcgacctggagaaggccgtggccaagggcgagttccgggccgacctgtattaccgtatcaatgtggtcagcatcttcattcctccgctgcgcgaacggcgcgaggatattccctatctggtggaacacttcctggaaaaattccgtgtggagaaccagcgcgccatggtggccatgtcaccgcaggccatgaaggtcatgatgaattgctactggccgggcaatgtacgggaactggaaaattgtgtcgagcgtactgccaccatgatgcgcggcgatctgatcaccgaggtgcatttctcctgtcagcagaacaaatgcctgaccaaggtcttgcatgaacccggccagcagcaaccggtcgtggtggtgccgctggagcgcatatccgcgcatacggcgccatcttcgccgagtgggatggccaaggacaagccactggcggccgcgcccccgacctccgaacgcgaacgtcttatctgggccatggagcaatgtggctgggtgcaagccaaggcagcccgcgcactcaacatctcgccgcgccagatgggctatgcgctgcagaaattcaatatcgaggtcaagaagttctaataaatcccatcaaataaaacgaaaggctcagtcgaaagactgggcctttcgttttatctgttgttttcctctgccgtactgatcgactgagtctgcagtattccttcggggccggcgacggcctctttcaaaggcgccttcgggcgccttttttcattgcctggatgcaagctgcttttttctgtggcattcgcgcgtcgccccggataattcccgactatttgagtcggactctttaaaatccctccatcatcgccatctgaacactcccagcgacgctttcgcagcgggcatcgccagggccggcctttgccggaatggcaacagtggcttgtgtcgcgtcgcaacacgatgtcaggaatccgctaaaatcgatcctcgctgccggcatttacatatcgttaaggtctcttatggtcccgcatctcgtcaccgctctgaatggtcctctgctggagctggagaagaaaatcctgggcgccacgccggccatcgaacgctggttccgcatggaatggcaggagcacacgccgcccttctattgctcggtcgacctgcgcaacgccggcttcaagctggcgccggtcgacaccaacctgttcccgggcggcttcaacaacctttctcccgagatgctgccgctggcggtgcaggcggccatggccgccatcgagaaatactgtcccgatgcccgcaacctgctgctgatcccggaaaaccacaccggcaacaccttctacctgcagaacatcgcccggctgatgcagatcttccgccagaccgggctgaacgtgcgcctgggcagcctggcgccgggcgtgaacaagcccaagccgatcgaactgcccgatggcaccaccatcaccctggagccgctggagcgctcggccaacggccgccgcctgggactgaagaacttcgatccctgtacggtgctgctcaacaatgacctgtccatcggcatcccgtcggtgctggaaggcttgcatgagcagagcgtgctgccgccgttgcacgccggctgggcgatgcgccgcaagagcaaccatttcgccgcctatgatgaggtggtcaagaagtttgccaagatggtcgaggtcgatccctggatgctcaatccgctctttgccaaggtcagcgacatcgacttccaggaaagcaccggcaccgatgcgctggccgacagcgtttcttcgctgttgtccaagatccgcaagaagtacaaggaatacggtatcaaggagcagcccttcgtcttcgtcaaggccaacgccggcacctatggcatgggcatcatgacggtcaaggacgccagcgagatcaaggacctgaaccgcaagcagcgcaccggcatgctggcggccggcaaggaagggctggaggtgtcggaggtgatcgtgcaggaaggcgtgcattccttcgagcgcatcaaccaggccgtggccgagccggtggtctacatgatcgaccgctatgtggtgggcggcttctatcgcgtccacgccgaccgtgacgtcaacgagaacctcaacgcacccggcgcgcatttcgtgccgctggccttcgcccagcagcacgccctgccggacatgcatgccaagccgggcacggcggcgcccaaccgtttctacatgtatggcgtggtggcgcgattggcgctgctggcggcctcggtggaaatggaaaagaccgatcccaacccggaagtttattaatatcgggccttcgccaggctcaacaggaccctagcctcatgaaaatcgctttcctcgccgatccactggatacgttcaagacgtacaaggattccaccttcgccatgatgcgcgaagccgcgcgccgtggccacgtcctctacgccttcgaacaggccgacctggtgctggaaagcgggctggtgacggccaacgcctgcaagctcacgctcacgggcgatgccgatgactggtatcggaccgacaagccggtctcttctttcctgtccgaattcgatgcggtcatcgtgcgcaaggacccgcccttcgacatggagtacatctacacgacctacctgctggagctggccgcctccttacgcagggcatccatttattactcaaccgtaaccgattttgccaggttacgcggctggtcctctagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcgtcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagcctttgctggccttttgctcacatgttctttcctgcgttatcaggggattccttaaggtatactttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccgataagctctgataccgctcgccgcagccgaacgaccgagcgcagcgggtcagtgagcgaggaagcggaagagcgcccaatacgcaaaccgcctctccccgcgcgttggccgattcattaatgcagcgcccaaacataacaggaagaaaaatgccccg

SEQ ID NO: 13 - DNA, 10704 bp, synthetic construct
  misc_feature 1..10704: note = amtB upstream homology arm - CP32 promoter - Hs NifA CDS - amtB downstream homology arm in vector including gentamicin selectable marker and human herpes virus 1 thymidine kinase counter-selectable marker kinase counter-selectable marker
  source 1..10704: mol_type = other DNA
ggaccgaaacaaaaaaagacgcttttcagcgtcttcaataattggtttggtaccgagttatagtccagggcgttactttagttggcttctcccatctcgcgtgcaaacgtgcgcgctaaatcacagatggtcggaatcgagcccggagtggtgacatgggtctgcaccatgccagaggtcagttgtagcagcgcatctctgcaaccagctggcgactgatcgtaatccaggataaatacgtgcattgagcgcagtcttttagccagaacgtccagggcccaggcgaacacgttatacaagtctccattcggggcgagaagttcgggtgcacgaaacaacgtgaacagtgtatcccctatatgagggcgagggcctgcgttggactgaggttccgcaccttgaggagggacggccgtacctgagagctggccccaatcctctcgccagctaccaccacattgaagataacgaactgtattcgccagtaagccatacacgcgacggatcgcagctagcatggccaagtctaacctctcccccggacgctggcgtttagccagacgatcgatatgtcgatcttctggcaatgcgcccagcacaatgttggtcccggggagcgtagggggaataagcgcaacaaatgccagaacagcttgcggagtcatactacccatcaagtaacgcgcggcaggataacacagtaacgcggcaattgggtgacggtcgaagatgagtgtcagtgccggaggtggagcatggctggagcccgcttctcccccaatgtgcggggctagtacagcatcagtgacagcgtacggcatacccatggtgatctgcgccgaggtcatgaccactgcggcatcgccagcagagatttcaccctgatccagtcgatgttgcgtagtataaatattcgctatcgtttcgctggcgcccagaactctccagtaggtcattggttccggcacatacacgatgtcatcgcgggaacccagcgctaccaggagctgggtcgtggtagttttgcccatcccgtgcggtccatcaatataaacccgaagtaaggttggcatcttctgttcggggcggacctccgttgcttcttgctggcgtcgcggacgcaaggctgtacggcggttagaatggcccctactccgcgcagcttggtcaaaggctgaggcatgctgatgaccagggtatgaagccattatttcctccttacgtctaggaacgctagcataatacctaggactgagctagctgtaaaaagatcccctgattccctttgtcaacagcaatggatcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggtgacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcggccaattcgatctagaattccattgagtaagtttttaagcacatcagcttcaaaagcgctctgggcgaaggagggcaggccattggccagatgcacctccaccgtgaccggtggggcgttcatgccggccagggcgcggctgcgcaggaccgccaggctcatcgcgatgggcctggctgcctgttacctatatagagaagagagtgtgatcgaagtgaaagtaggtgcgccaggatcatggagtctgtttttgctgggcttccagctcggccacacgggcctccagggcttccaggcgggcgcgggtcttggccagcacctgagcctggatgtcgaattcctcgcgcgtgaccaggtcgagcttggcaaagccctggctgagcatggccttgacgttcttttcgatgtccttggccggggagttttcgattgccttgttgatcttggattgcaggtcttcaaaaaacgggggcttgtccatgtcggatctccttggcagataagctgtagctggaatgcaagtttagcggtttcctgcccatcctcaaaccgccttgcctggatgagaccgcatttttaccgccaagtgcggcctgtgaagttttttccttcaggaaatgcgccgtcttggtgatccggaacgcccctgctggccgcgcaccaaaatgtgattgccggaatggaagcagggttaacaaaaaacaagcaatatcgcaccaatcacgtgcattttcccttggtctgcactattttgaactgaagttagaaaaaaaacgtgaccaatctccggtttcacctaaaaacacaagcctgccttgccgaaagaacatggcacggctcctgctacagggtttgagcaaacacaaaaaacgacttttattgttcaaacacaaaccagagggaaagtcatgaagaaactgattcttgcaacagccatcgccgcatccttcgtttctaccgtggcccgtgcagaagacgccaaaccagataatgagttcagcttcaacgccgctgtcgtgagcgactaccgctaccgcggcctgacccagacccgcttcgacccggccctgcaagtcggtgctgactacacccacaaccccaccggcctgtacgccggcacctggctgaccaacatcaagtggatcaaggacgctggcgccgtggccagcccgccggtcaacaccaagggcgacatcgagtgggacatctatggcggcaagcgcggcgacatcggcggcggcttcacctatgacgtcggcggcctgtactactactacccgggcaacaccctggccgacagcggcgccaagaacgccaacaccttcgaactgtacggccaggtcggctacggtccgggctacttcaagtattcgcaatcgctgaccaacctgttcggcgcagccaacagcaagtacagccagtacttcgacctgggcgccaacatcgagacctactacggcgtcatcctgggcctgcacgtgggtcaccagaacgtgcgcggcaaccccaaccagttcagctacaccgactggaagatcggcgtgtccaagaccttcgacgatctggccggcatcaccctgggcctggcctatgtcggcaccgacctcaaggatggcgtgaccaccacgccggcttcggatggcctgaagaacgtcggcaagaacggcttcgttttctccctcagcaaaactttctaacaagaggttcgtatgaaactgatcacagcgatcatcaaaccgttcaagctggacgaggtgcgtgaagccctctccgcgatcggggtgcaaggcatcaccgtgactgaagtcaagggcttcggtcgtcagaagggccataccgagctgtaccgcggcgcggaatacgtggtcgatttcctgcccaagaccaagatcgaggcggcggtggacgacgccatcgtcgaacgcgcgctcgaagccatcgagaccgctgcccgcaccggcaagatcggcgacggcaagatcttcgtccaggacctgctggacgtgatccgtatccgcaccggcgagaccggcaaagacgctctgtaaaaaaggggaactctacatacgggagtttattcttgacatattgccggtgtgttggtataataacttaagtcaataaaggcatataaaaggaggttaataacatgaaagttaaagtaaaacatcttaatcatgcggtggagggtttctaatggccactattctcgacgaccgcagtgtgaatttagaactcgtaacgatttacgagatcagcaagatattgggttcatcgctggatctatccaagaccttgcgcgaagtcctcaacgtcctctccgcccacctggaaacgaaacgggtattgctgagtctgatgcaggactccggtgaattgcaactggtcagcgccatcggcctgagttacgaagaattccagagtggcagataccgggtcggcgagggcatcaccggcaagatcttccagacggaaactcccatcgtggtgcgcgacttggcccaggagccgctgttcctggctcgcaccagcccgcgccagtcgcaagacggggaagtcatttcctttgtgggcgtgccgatcaaggctgcccgtgaaatgctgggtgtgttgtgcgtttttcgggatggacaatcgccctcgcgtagtgtcgaccatgaggtgcgcctgctgaccatggtggccaacctgatcggacaaaccgtccggctgtatcgcagcgtagcggccgagcgccagcagctgcaagaggaaaagcgccagctgtcccggcaactccagggcaagtacaagctcgataacgtaatcggcatttccaaggcgatgcaggaagtgttcgcgcaggtacaccagtccgccccctcgcgctcgaccatgctgttgcgcggcgagagcggcaccggcaaggaagtcatcgcgcgcgccatccattacctcagtccgcgcaaggatgggccattcatcaaggtcaactgtgcggccttgtcggaaaccttgctggaatccgaattgtttggccacgagaagggcgccttcaccggggcccagggtgaacgcaaggggcgtttcgaactcgcccatggcggcaccttgttcctcgacgagatcggcgagatctcgccggccttccaggccaagctgctgcgggtattgcaggagcgtgaattcgagcgggtcggtgggtcgcgttcgatcaaggtggacgtgcgtctggtcaccgccaccaaccgcgacctggagaaggccgtggccaagggcgagttccgggccgacctgtattaccgtatcaatgtggtcagcatcttcattcctccgctgcgcgaacggcgcgaggatattccctatctggtggaacacttcctggaaaaattccgtgtggagaaccagcgcgccatggtggccatgtcaccgcaggccatgaaggtcatgatgaattgctactggccgggcaatgtacgggaactggaaaattgtgtcgagcgtactgccaccatgatgcgcggcgatctgatcaccgaggtgcatttctcctgtcagcagaacaaatgcctgaccaaggtcttgcatgaacccggccagcagcaaccggtcgtggtggtgccgctggagcgcatatccgcgcatacggcgccatcttcgccgagtgggatggccaaggacaagccactggcggccgcgcccccgacctccgaacgcgaacgtcttatctgggccatggagcaatgtggctgggtgcaagccaaggcagcccgcgcactcaacatctcgccgcgccagatgggctatgcgctgcagaaattcaatatcgaggtcaagaagttctgacctctgccgtactgatcgactgagtctgcagtattccttcggggccggcgacggcctctttcaaaggcgccttcgggcgccttttttcattgcctggatgcaagctgcttttttctgtggcattcgcgcgtcgccccggataattcccgactatttgagtcggactctttaaaatccctccatcatcgccatctgaacactcccagcgacgctttcgcagcgggcatcgccagggccggcctttgccggaatggcaacagtggcttgtgtcgcgtcgcaacacgatgtcaggaatccgctaaaatcgatcctcgctgccggcatttacatatcgttaaggtctcttatggtcccgcatctcgtcaccgctctgaatggtcctctgctggagctggagaagaaaatcctgggcgccacgccggccatcgaacgctggttccgcatggaatggcaggagcacacgccgcccttctattgctcggtcgacctgcgcaacgccggcttcaagctggcgccggtcgacaccaacctgttcccgggcggcttcaacaacctttctcccgagatgctgccgctggcggtgcaggcggccatggccgccatcgagaaatactgtcccgatgcccgcaacctgctgctgatcccggaaaaccacaccggcaacaccttctacctgcagaacatcgcccggctgatgcagatcttccgccagaccgggctgaacgtgcgcctgggcagcctggcgccgggcgtgaacaagcccaagccgatcgaactgcccgatggcaccaccatcaccctggagccgctggagcgctcggccaacggccgccgcctgggactgaagaacttcgatccctgtacggtgctgctcaacaatgacctgtccatcggcatcccgtcggtgctggaaggcttgcatgagcagagcgtgctgccgccgttgcacgccggctgggcgatgcgccgcaagagcaaccatttcgccgcctatgatgaggtggtcaagaagtttgccaagatggtcgaggtcgatccctggatgctcaatccgctctttgccaaggtcagcgacatcgacttccaggaaagcaccggcaccgatgcgctggccgacagcgtttcttcgctgttgtccaagatccgcaagaagtacaaggaatacggtatcaaggagcagcccttcgtcttcgtcaaggccaacgccggcacctatggcatgggcatcatgacggtcaaggacgccagcgagatcaaggacctgaaccgcaagcagcgcaccggcatgctggcggccggcaaggaagggctggaggtgtcggaggtgatcgtgcaggaaggcgtgcattccttcgagcgcatcaaccaggccgtggccgagccggtggtctacatgatcgaccgctatgtggtgggcggcttctatcgcgtccacgccgaccgtgacgtcaacgagaacctcaacgcacccggcgcgcatttcgtgccgctggccttcgcccagcagcacgccctgccggacatgcatgccaagccgggcacggcggcgcccaaccgtttctacatgtatggcgtggtggcgcgattggcgctgctggcggcctcggtggaaatggaaaagaccgatcccaacccggaagtttattaatatcgggccttcgccaggctcaacaggaccctagcctcatgaaaatcgctttcctcgccgatccactggatacgttcaagacgtacaaggattccaccttcgccatgatgcgcgaagccgcgcgccgtggccacgtcctctacgccttcgaacaggccgacctggtgctggaaagcgggctggtgacggccaacgcctgcaagctcacgctcacgggcgatgccgatgactggtatcggaccgacaagccggtctcttctttcctgtccgaattcgatgcggtcatcgtgcgcaaggacccgcccttcgacatggagtacatctacacgacctacctgctggagctggccgcctccttacgcagggcatccatttattactcaaccgtaaccgattttgccaggttacgcggctggtcctctagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagggcctcgtgatacgcctatttttataggttaatgtcatgataataatggtttcttagacgtcaggtggcacttttacagccttacgcatctgtgcggtatttcacaccgcatatggtgcactctcagtacaatctgctctgatgccgcatagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagcctttgctggccttttgctcacatgttctttcctgcgttatcaggggattccttaaggtatactttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccgataagctctgataccgctcgccgcagccgaacgaccgagcgcagcgggtcagtgagcgaggaagcggaagagcgcccaatacgcaaaccgcctctccccgcgcgttggccgattcattaatgcagcgcccaaacataacaggaagaaaaatgccccg

SEQ ID NO: 14 - DNA, 8885 bp, synthetic construct
  misc_feature 1..8885: note = amtB upstream homology arm - amtB downstream homology arm in vector including gentamicin selectable marker and human herpes virus 1 thymidine kinase counter-selectable marker
  source 1..8885: mol_type = other DNA
ggaccgaaacaaaaaaagacgcttttcagcgtcttcaataattggtttggtaccgagttatagtccagggcgttactttagttggcttctcccatctcgcgtgcaaacgtgcgcgctaaatcacagatggtcggaatcgagcccggagtggtgacatgggtctgcaccatgccagaggtcagttgtagcagcgcatctctgcaaccagctggcgactgatcgtaatccaggataaatacgtgcattgagcgcagtcttttagccagaacgtccagggcccaggcgaacacgttatacaagtctccattcggggcgagaagttcgggtgcacgaaacaacgtgaacagtgtatcccctatatgagggcgagggcctgcgttggactgaggttccgcaccttgaggagggacggccgtacctgagagctggccccaatcctctcgccagctaccaccacattgaagataacgaactgtattcgccagtaagccatacacgcgacggatcgcagctagcatggccaagtctaacctctcccccggacgctggcgtttagccagacgatcgatatgtcgatcttctggcaatgcgcccagcacaatgttggtcccggggagcgtagggggaataagcgcaacaaatgccagaacagcttgcggagtcatactacccatcaagtaacgcgcggcaggataacacagtaacgcggcaattgggtgacggtcgaagatgagtgtcagtgccggaggtggagcatggctggagcccgcttctcccccaatgtgcggggctagtacagcatcagtgacagcgtacggcatacccatggtgatctgcgccgaggtcatgaccactgcggcatcgccagcagagatttcaccctgatccagtcgatgttgcgtagtataaatattcgctatcgtttcgctggcgcccagaactctccagtaggtcattggttccggcacatacacgatgtcatcgcgggaacccagcgctaccaggagctgggtcgtggtagttttgcccatcccgtgcggtccatcaatataaacccgaagtaaggttggcatcttctgttcggggcggacctccgttgcttcttgctggcgtcgcggacgcaaggctgtacggcggttagaatggcccctactccgcgcagcttggtcaaaggctgaggcatgctgatgaccagggtatgaagccattatttcctccttacgtctaggaacgctagcataatacctaggactgagctagctgtaaaaagatcccctgattccctttgtcaacagcaatggatcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcggccaattcgatctagaattccattgagtaagtttttaagcacatcagcttcaaaagcgctctgggcgaaggagggcaggccattggccagatgcacctccaccgtgaccggtggggcgttcatgccggccagggcgcggctgcgcaggaccgccaggctcatcgcgatgggcctggctgcctgttacctatatagagaagagagtgtgatcgaagtgaaagtaggtgcgccaggatcatggagtctgtttttgctgggcttccagctcggccacacgggcctccagggcttccaggcgggcgcgggtcttggccagcacctgagcctggatgtcgaattcctcgcgcgtgaccaggtcgagcttggcaaagccctggctgagcatggccttgacgttcttttcgatgtccttggccggggagttttcgattgccttgttgatcttggattgcaggtcttcaaaaaacgggggcttgtccatgtcggatctccttggcagataagctgtagctggaatgcaagtttagcggtttcctgcccatcctcaaaccgccttgcctggatgagaccgcatttttaccgccaagtgcggcctgtgaagttttttccttcaggaaatgcgccgtcttggtgatccggaacgcccctgctggccgcgcaccaaaatgtgattgccggaatggaagcagggttaacaaaaaacaagcaatatcgcaccaatcacgtgcattttcccttggtctgcactattttgaactgaagttagaaaaaaaacgtgaccaatctccggtttcacctaaaaacacaagcctgccttgccgaaagaacatggcacggctcctgctacagggtttgagcaaacacaaaaaacgacttttattgttcaaacacaaaccagagggaaagtcatgaagaaactgattcttgcaacagccatcgccgcatccttcgtttctaccgtggcccgtgcagaagacgccaaaccagataatgagttcagcttcaacgccgctgtcgtgagcgactaccgctaccgcggcctgacccagacccgcttcgacccggccctgcaagtcggtgctgactacacccacaaccccaccggcctgtacgccggcacctggctgaccaacatcaagtggatcaaggacgctggcgccgtggccagcccgccggtcaacaccaagggcgacatcgagtgggacatctatggcggcaagcgcggcgacatcggcggcggcttcacctatgacgtcggcggcctgtactactactacccgggcaacaccctggccgacagcggcgccaagaacgccaacaccttcgaactgtacggccaggtcggctacggtccgggctacttcaagtattcgcaatcgctgaccaacctgttcggcgcagccaacagcaagtacagccagtacttcgacctgggcgccaacatcgagacctactacggcgtcatcctgggcctgcacgtgggtcaccagaacgtgcgcggcaaccccaaccagttcagctacaccgactggaagatcggcgtgtccaagaccttcgacgatctggccggcatcaccctgggcctggcctatgtcggcaccgacctcaaggatggcgtgaccaccacgccggcttcggatggcctgaagaacgtcggcaagaacggcttcgttttctccctcagcaaaactttctaacaagaggttcgtatgaaactgatcacagcgatcatcaaaccgttcaagctggacgaggtgcgtgaagccctctccgcgatcggggtgcaaggcatcaccgtgactgaagtcaagggcttcggtcgtcagaagggccataccgagctgtaccgcggcgcggaatacgtggtcgatttcctgcccaagaccaagatcgaggcggcggtggacgacgccatcgtcgaacgcgcgctcgaagccatcgagaccgctgcccgcaccggcaagatcggcgacggcaagatcttcgtccaggacctgctggacgtgatccgtatccgcaccggcgagaccggcaaagacgctctgtaaaaaaggggaactctacctctgccgtactgatcgactgagtctgcagtattccttcggggccggcgacggcctctttcaaaggcgccttcgggcgccttttttcattgcctggatgcaagctgcttttttctgtggcattcgcgcgtcgccccggataattcccgactatttgagtcggactctttaaaatccctccatcatcgccatctgaacactcccagcgacgctttcgcagcgggcatcgccagggccggcctttgccggaatggcaacagtggcttgtgtcgcgtcgcaacacgatgtcaggaatccgctaaaatcgatcctcgctgccggcatttacatatcgttaaggtctcttatggtcccgcatctcgtcaccgctctgaatggtcctctgctggagctggagaagaaaatcctgggcgccacgccggccatcgaacgctggttccgcatggaatggcaggagcacacgccgcccttctattgctcggtcgacctgcgcaacgccggcttcaagctggcgccggtcgacaccaacctgttcccgggcggcttcaacaacctttctcccgagatgctgccgctggcggtgcaggcggccatggccgccatcgagaaatactgtcccgatgcccgcaacctgctgctgatcccggaaaaccacaccggcaacaccttctacctgcagaacatcgcccggctgatgcagatcttccgccagaccgggctgaacgtgcgcctgggcagcctggcgccgggcgtgaacaagcccaagccgatcgaactgcccgatggcaccaccatcaccctggagccgctggagcgctcggccaacggccgccgcctgggactgaagaacttcgatccctgtacggtgctgctcaacaatgacctgtccatcggcatcccgtcggtgctggaaggcttgcatgagcagagcgtgctgccgccgttgcacgccggctgggcgatgcgccgcaagagcaaccatttcgccgcctatgatgaggtggtcaagaagtttgccaagatggtcgaggtcgatccctggatgctcaatccgctctttgccaaggtcagcgacatcgacttccaggaaagcaccggcaccgatgcgctggccgacagcgtttcttcgctgttgtccaagatccgcaagaagtacaaggaatacggtatcaaggagcagcccttcgtcttcgtcaaggccaacgccggcacctatggcatgggcatcatgacggtcaaggacgccagcgagatcaaggacctgaaccgcaagcagcgcaccggcatgctggcggccggcaaggaagggctggaggtgtcggaggtgatcgtgcaggaaggcgtgcattccttcgagcgcatcaaccaggccgtggccgagccggtggtctacatgatcgaccgctatgtggtgggcggcttctatcgcgtccacgccgaccgtgacgtcaacgagaacctcaacgcacccggcgcgcatttcgtgccgctggccttcgcccagcagcacgccctgccggacatgcatgccaagccgggcacggcggcgcccaaccgtttctacatgtatggcgtggtggcgcgattggcgctgctggcggcctcggtggaaatggaaaagaccgatcccaacccggaagtttattaatatcgggccttcgccaggctcaacaggaccctagcctcatgaaaatcgctttcctcgccgatccactggatacgttcaagacgtacaaggattccaccttcgccatgatgcgcgaagccgcgcgccgtggccacgtcctctacgccttcgaacaggccgacctggtgctggaaagcgggctggtgacggccaacgcctgcaagctcacgctcacgggcgatgccgatgactggtatcggaccgacaagccggtctcttctttcctgtccgaattcgatgcggtcatcgtgcgcaaggacccgcccttcgacatggagtacatctacacgacctacctgctggagctggccgcctccttacgcagggcatccatttattactcaaccgtaaccgattttgccaggttacgcggctggtcctctagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcgtcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgactcgagcgtcgatttttgtgatgctcgtcaggggggcggagcctatggaaaaacgccagcaacgcggcctttttacggttcctggccttttgctggccttttgctcacatgttctttcctgcgttatcaggggattccttaaggtatactttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccgataagctctgataccgctcgccgcagccgaacgaccgagcgcagcgggtcagtgagcgaggaagcggaagagcgcccaatacgcaaaccgcctctccccgcgcgttggccgattcattaatgcagcgcccaaacataacaggaagaaaaatgccccg

SEQ ID NO: 15 - DNA, 9170 bp, synthetic construct
  misc_feature 1..9170: note = Hs NifA promoter upstream homology arm - CP25 promoter - Hs NifA promoter downstream homology arm in vector including gentamicin selectable marker and human herpes virus 1 thymidine kinase counter-selectable marker
  source 1..9170: mol_type = other DNA
ggaccgaaacaaaaaaagacgcttttcagcgtcttcaataattggtttggtaccgagttatagtccagggcgttactttagttggcttctcccatctcgcgtgcaaacgtgcgcgctaaatcacagatggtcggaatcgagcccggagtggtgacatgggtctgcaccatgccagaggtcagttgtagcagcgcatctctgcaaccagctggcgactgatcgtaatccaggataaatacgtgcattgagcgcagtcttttagccagaacgtccagggcccaggcgaacacgttatacaagtctccattcggggcgagaagttcgggtgcacgaaacaacgtgaacagtgtatcccctatatgagggcgagggcctgcgttggactgaggttccgcaccttgaggagggacggccgtacctgagagctggccccaatcctctcgccagctaccaccacattgaagataacgaactgtattcgccagtaagccatacacgcgacggatcgcagctagcatggccaagtctaacctctcccccggacgctggcgtttagccagacgatcgatatgtcgatcttctggcaatgcgcccagcacaatgttggtcccggggagcgtagggggaataagcgcaacaaatgccagaacagcttgcggagtcatactacccatcaagtaacgcgcggcaggataacacagtaacgcggcaattgggtgacggtcgaagatgagtgtcagtgccggaggtggagcatggctggagcccgcttctcccccaatgtgcggggctagtacagcatcagtgacagcgtacggcatacccatggtgatctgcgccgaggtcatgaccactgcggcatcgccagcagagatttcaccctgatccagtcgatgttgcgtagtataaatattcgctatcgtttcgctggcgcccagaactctccagtaggtcattggttccggcacatacacgatgtcatcgcgggaacccagcgctaccaggagctgggtcgtggtagttttgcccatcccgtgcggtccatcaatataaacccgaagtaaggttggcatcttctgttcggggcggacctccgttgcttcttgctggcgtcgcggacgcaaggctgtacggcggttagaatggcccctactccgcgcagcttggtcaaaggctgaggcatgctgatgaccagggtatgaagccattatttcctccttacgtctaggaacgctagcataatacctaggactgagctagctgtaaaaagatcccctgattccctttgtcaacagcaatggatcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcggccaattcgatctagaattccattgagtaagtttttaagcacatcagcttcaaaagcgctctgcgatggtgacggtgacttgcgtagtgacagggccggtatcgatcgcactgatgaccccggcgaattgattacgtgcgctggttttcattgacattctcctgagcaggccagccaaggctggcagttgattaagccatgtctcctgccgatgcaggaattcgagattgcgccgttgcagctcgcgccacacggccagcagttccaccgcggcgggcgtgagccgggtgccgccgccgcctttgccgccggtgaccgtttcaatcaatgcgccattgaccaggttgccggctgactccagcagcatccatgcgcccttgtagctgtagcccgccgtactggccgcgcgattgatcgagccggtttgcgcgatggcgtcgagcagggcaaagaagcgctggtccaggcgatgcaccagtttcagttcgctgctcaacaaggtggtatcggtggcaggcaaggtactcatgtccttgccgatggctgcagtcccagcgcgcccgccagcaagctcagggctgcgcgcgcttgctggggatcgtggctggcaggcttgagcagcaggcgatgcggccccagtttttcttgcagctggccgatgcgtgccacgcattgcgccgggtcgccgatgatggcgttggccaggatgacttgatcggaggcatcggcctggaacatgggcggggtgccaccgggcgcgaattgcacttgcatcatgcgcggatacgcacgcacacctgccagaccatgggcaagcgccttgtgcgcctcgggttcgcaatggaaaaagcgggcgatggcaaacgggaagtgaccgcgctgcgcacgctgtgcagccacccgcgtgagcacctgctcaagatcggcggagggtgtggccatcaatccgtaatcctggcgtgcggccagatcaagcgaatcttcgctcatgctggccagccataccgggatgggacgttgcagcggctgcggttgcaactgcactctgtcgtagtgaaaaaatcgtccctcggtactgaatggcacgccttgccatgcgcgctggatgacgtccagtgcttcaatcatgcgtgcgcgcgcctgatcggcaccgctcaatccggcgtgacgatattgctcgggaaacgggccgccgcgtgccacgccgaattccatccggccgttgctgagcaggtccagcgtggcgacgtcctcggcaacccgcaagggatcattgagcgccagcaacgaagcgcccgtaccgaggcggatgcgcgaagtgcgtgcggcgatctgggccagcagcaccatcaaggcgctgccgatggcgaagctgctgtaatggtgctcggcgacccagacatcgtgatagcccagggcctctgccacgacggcatgatcgacggtctgcaacagcgcatcgcgtgcgttgccatggggattttcgacattgaggaagacgccgtagcgttgcgggtaatccggcatggcaggttcacgggaaaaagagttggacatcgctcagaccggccgcatcaagaaacagatgcggggatgcgatgccggatggcgcaggtcatgattcggatcttcccgatggccggatcagatccgctatcggcggcagcatgtcggtggcgggcgagtggaaccagctgcgtaccagctcataggggtagcgccgcggcggttcgacgcgcgcatgacgtaccagcagatgaatcgacaagggttcggcccaggcccagaagtcggcccgcagcgcgggcgggaaaccgatatcgtcaaaggtatgccaaagctgtaccagccacagttcgcgcccagcttcgtccaatagcaacggcaagcctgcgcccgcttgcagatgggcatggcgttggctgtagtagagcggaccgccgcatgactccaccacgaagtcggcgatgcgcgagctgacgcaatcaaaacaattgtgcgaacgtgcgaacagcggggaatggcgcaggcgccgcatatgatgcagcaccagtttgcgcaagccggattcatcacacagcgccagcaaccggtgcgccggttgaggtaattgtggcggctcacatatcacctgttgcgtgccgcgcgccatcgccgggccggcgaccggccagttcagggtcagataacatgcctgggagcagggcgcccactttggcagtttattcttgacatgtagtgagggggctggtataatcacataagtcaataaaggcatataaaaggaggttaataacatgaaagttaaagtaaaacatcttaatcatgcggtggagggtttctaatgatggccactattctcgacgaccgcagtgtgaatttagaactcgtaacgatttacgagatcagcaagatattgggttcatcgctggatctatccaagaccttgcgcgaagtcctcaacgtcctctccgcccacctggaaacgaaacgggtattgctgagtctgatgcaggactccggtgaattgcaactggtcagcgccatcggcctgagttacgaagaattccagagtggcagataccgggtcggcgagggcatcaccggcaagatcttccagacggaaactcccatcgtggtgcgcgacttggcccaggagccgctgttcctggctcgcaccagcccgcgccagtcgcaagacggggaagtcatttcctttgtgggcgtgccgatcaaggctgcccgtgaaatgctgggtgtgttgtgcgtttttcgggatggacaatcgccctcgcgtagtgtcgaccatgaggtgcgcctgctgaccatggtggccaacctgatcggacaaaccgtccggctgtatcgcagcgtagcggccgagcgccagcagctgcaagaggaaaagcgccagctgtcccggcaactccagggcaagtacaagctcgataacgtaatcggcatttccaaggcgatgcaggaagtgttcgcgcaggtacaccagtccgccccctcgcgctcgaccatgctgttgcgcggcgagagcggcaccggcaaggaagtcatcgcgcgcgccatccattacctcagtccgcgcaaggatgggccattcatcaaggtcaactgtgcggccttgtcggaaaccttgctggaatccgaattgtttggccacgagaagggcgccttcaccggggcccagggtgaacgcaaggggcgtttcgaactcgcccatggcggcaccttgttcctcgacgagatcggcgagatctcgccggccttccaggccaagctgctgcgggtattgcaggagcgtgaattcgagcgggtcggtgggtcgcgttcgatcaaggtggacgtgcgtctggtcaccgccaccaaccgcgacctggagaaggccgtggccaagggcgagttccgggccgacctgtattaccgtatcaatgtggtcagcatcttcattcctccgctgcgcgaacggcgcgaggatattccctatctggtggaacacttcctggaaaaattccgtgtggagaaccagcgcgccatggtggccatgtcaccgcaggccatgaaggtcatgatgaattgctactggccgggcaatgtacgggaactggaaaattgtgtcgagcgtactgccaccatgatgcgcggcgatctgatcaccgaggtgcatttctcctgtcagcagaacaaatgcctgaccaaggtcttgcatgaacccggccagcagcaaccggtcgtggtggtgccgctggagcgcatatccgcgcatacggcgccatcttcgccgagtgggatggccaaggacaagccactggcggccgcgcccccgacctccgaacgcgaacgtcttatctgggccatggagcaatgtggctgggtgcaagccaaggcagcccgcgcactcaacatctcgccgcgccagatgggctatgcgctgcagaaattcaatatcgaggtcaagaagttctgaaactcatctgcgagcgtgacgtaatacgctgccagatccaaagtctttttctcattgccgtcctcgccctgcgcgcgcggtctcttcggcgtcgcgccagccggttgtattgatggattccccggctggcacgtcctcgcattctccttctccttctccactgcgtcaggctcattgcggcgcgctcaattgcaggcgccggcccgtgctgcattgtcgtgttcatgacaagcgccacccacaatgtatcgccgattgtcgctatcctcacatcgccgtacgacgcaattctccgctatgtcattgaattgattgattattttgtctggcacggttttggctatgtcctcaggagaaggcccaggcatcaccctccttacgcagggcatccatttattactcaaccgtaaccgattttgccaggttacgcggctggtcctctagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcgtcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgactcgagcgtcgatttttgtgatgctcgtcaggggggcggagcctatggaaaaacgccagcaacgcggcctttttacggttcctggccttttgctggccttttgctcacatgttctttcctgcgttatcaggggattccttaaggtatactttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccgataagctctgataccgctcgccgcagccgaacgaccgagcgcagcgggtcagtgagcgaggaagcggaagagcgcccaatacgcaaaccgcctctccccgcgcgttggccgattcattaatgcagcgcccaaacataacaggaagaaaaatgccccg

SEQ ID NO: 16 - DNA, 8886 bp, synthetic construct
  misc_feature 1..8886: note = ntrC upstream homology arm - ntrC downstream homology arm in vector including gentamicin selectable marker and human herpes virus 1 thymidine kinase counter-selectable marker
  source 1..8886: mol_type = other DNA
ggaccgaaacaaaaaaagacgcttttcagcgtcttcaataattggtttggtaccgagttatagtccagggcgttactttagttggcttctcccatctcgcgtgcaaacgtgcgcgctaaatcacagatggtcggaatcgagcccggagtggtgacatgggtctgcaccatgccagaggtcagttgtagcagcgcatctctgcaaccagctggcgactgatcgtaatccaggataaatacgtgcattgagcgcagtcttttagccagaacgtccagggcccaggcgaacacgttatacaagtctccattcggggcgagaagttcgggtgcacgaaacaacgtgaacagtgtatcccctatatgagggcgagggcctgcgttggactgaggttccgcaccttgaggagggacggccgtacctgagagctggccccaatcctctcgccagctaccaccacattgaagataacgaactgtattcgccagtaagccatacacgcgacggatcgcagctagcatggccaagtctaacctctcccccggacgctggcgtttagccagacgatcgatatgtcgatcttctggcaatgcgcccagcacaatgttggtcccggggagcgtagggggaataagcgcaacaaatgccagaacagcttgcggagtcatactacccatcaagtaacgcgcggcaggataacacagtaacgcggcaattgggtgacggtcgaagatgagtgtcagtgccggaggtggagcatggctggagcccgcttctcccccaatgtgcggggctagtacagcatcagtgacagcgtacggcatacccatggtgatctgcgccgaggtcatgaccactgcggcatcgccagcagagatttcaccctgatccagtcgatgttgcgtagtataaatattcgctatcgtttcgctggcgcccagaactctccagtaggtcattggttccggcacatacacgatgtcatcgcgggaacccagcgctaccaggagctgggtcgtggtagttttgcccatcccgtgcggtccatcaatataaacccgaagtaaggttggcatcttctgttcggggcggacctccgttgcttcttgctggcgtcgcggacgcaaggctgtacggcggttagaatggcccctactccgcgcagcttggtcaaaggctgaggcatgctgatgaccagggtatgaagccattatttcctccttacgtctaggaacgctagcataatacctaggactgagctagctgtaaaaagatcccctgattccctttgtcaacagcaatggatcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcggccaattcgatctagaattccattgagtaagtttttaagcacatcagcttcaaaagcgctctgaatgtcgctattagagtgcaatgcgcatctctagcccggtatgccatgaagacgtcgcccgacgacatcgccgaaacgcccaatatcccatccacagagatgactgcttcgccgatgcgatgccaggagcttgctgctggccgaggtgcgctgccgcaaggccatccatgccggacctccgtcatccactgcgcaggccgcctccaatgcgctctgccctttcccctcaggtcaaatcgtgaacatcagaaataccaaagtcgccactcagctcatgctgggcttttccatcctgatcctcttcatggctgcgctgggaggaacgtccctctacatcatgggcaagatcaacgcccagctggtcgacgtccagggcaactggatgcccagcgtgactaccgcgcaaaccatgctggccgagatgcgggcggtcaacctggcccagtaccgcgccctgaccgccgacaatgcgcagcgcatgcaagacgcccgcacccgcatcaccgctgccctgggacgctacaccaaggccagcgagcagtaccgccacctcatcagctcgccggccgaacagagcgcctacgatgaactgcaggccttgctggcccgctatatccagatcggcgaccagttgccgcaaagcgtggagcaaggcaaggaagaagaagccgccaacaccatgaaggacacgctctatccggtgcgtaccgccatggaagccaagatccagcagatcatcaagatcaaccaggaaggcgcggcgcaggcggtcgaacacggccaggccagctatgcgcaaagccacctcaccatcatcgccttcaccctggccgcggcactggcgggactgggcattgcgctgctgatcagccgccgcctcacgcgccagctcggaggtgaaccgggcgaggcgatgtggctgtcgggccagatcgccgccggcaacctggcggcgcggctggaactcaagagcaatgacaacggcagcctgatgcgctcgctgatgatcatgcgcgaccagctcagcggcctggtgggccagatccagcatgccagcacgtccatccacgtggctgcccaggagatcgcgcagggcaacatcggtctctcgcagcgcaccgaagaacaggccgcctcgctggaagaaaccgcctccagcatggaagaactgaccagcactgtgcaacagaatgccggtaacgccaagcaggccagcagcatggccagcgccggtgcagaagtggcgcgccggggcgaggccgagatcgccgacgtggtggcgacgatgcgcgagatgagcgtgagctcgcaccagatgtcggacatcatcagcgtcatcgaaggcatttccttccagaccaatatcctggccctcaacgctgccgtggaagcggcccgcgccggcgagaacggacgcggcttcgccgtggtcgcctcggaagtgcgcgcgctggcccagcgcagctcggccgcggccaaggaaatcaaggacctgatcgaagactcggtggggcgcatccgcaacggctcccaactggtcgaagcggccggcgcgacgattgccgagatcatgcagtccagcgtgcgcaccaccgacctgatgaaggaaattgccgcggcttccgaggaacagagcgccggcatcggccaggtcaatacggccatcgtgcagatggaccaggtcacgcagcaaaacgcggcgctggtggaagaagcggcggcagccgcgcagtccatgatgcaacaggcgcaagcgctcacagcggcggtgtcggtcttcaagctggatgatcaggccttgcgtaccctgcccggcatgcccgccgcgccccagctgggccgttcactggccacgccggcgctgtcttgacggccgccgcaatagcggcgccgaggcaatgaacaatgaacaatgaacaatgcaaatggcctggatcgcaagatccaggccatttgtttttccagccacgcgcggcctggaacccgccctgccctgtgaggcatcacgtcttcctgcgttggtcttgctgttgatgctcccgcgagccaggccgcttctggcgccctgcccgcagcccccttggatgacgggatgtcttccgtcatgtcggctttggcgcgccgcctgcggccatgctcacggcagcggtataagtattctgaaatcggtacaacctggccggctgtcgcattcgatcacgcccatgtgctgatggacgaatgtctgcgccagggtcagccccagtccgctgccaccttcccgacccgacaccagtggatagaagattctgtcctggatgtcgggggggatgcccggaccgttatcgatgatatgcaagtctagtgccagccggtatcggaccttggatagcgtcacttgacggctcacgcgagtacgcaggatgagctcgccatcgccggcgcggatgcggtcggccagcgcctgggcggcgttgtgcacgatgttgagcacggcctggatgagctgctccttgtcgccgcgaaattccgggatggacaggtcatagtcgcgcttgatggtcagcccctgggggaactcggccatgaccaggctgcgcacgcgttcacagacttcgtggatgttgacgtcgccgacgatgtgcggatgccggtgcggcgccagcaggcgatcgaccagggtctgcaggcgatcggcttccttgatgatgacctgggtatattcgcgcaattccttgaggtgccgctcgggcagctccagctccagcagctgcgctgcgccgcggatgccgcccagcggattcttgatctcgtgggccaggttgcggatcagttccttgttggcctggctctggtcgaagatgcgctcttcgcggtccagcttcaattgctggacgttttcgcgcagctccagcagcaccggcatgtcggggttgtccagcggcgtgacgatggtgtggacctgcagcggttcgcggttggcgcgttccagcaccaggtccaggcgcttgtcggcgaactggtgttcgatggcctggtagaacagggccgagagctccttgccgttcaggaagagctccgagagcttctgctgcgagagcaccttgaaggagctttccagcaggttctcggcggcggcattggcatagacgatgccgccctcggcgtcgaggatgatcaccgccgaggccagcaggtcgagaccttcgagggaggggaacggagtcttcatggcggtagtcggggagcctggattcatgtcgtggagcagagtcgttcgtgcgggaacactagcaagtttcgggcgcgctcccatgaggatgcaaggaatgcacacaactcaggggacttgccggcgcgcataaaaaaaggggaagcaagcttcccctttttgtccggatgcggattcgattacagcgagtagtacatgtcgaattcgatcgggtgcgtggtcatgcggaagcgctggacttcctgcatcttcagatcgatgtaggcatcgatcatgctgtcggtgaacacgccgccacgggtcaggaactcgcggtccttgtccaggtgttccagcgcttcgtccagcgaggagcacacggtcgggatcagcttgtcttcttccggcggcaggtggtacaggtccttggtcgcagcttcgcccgggtggatcttgttctgcacgccatccagaccggccatcagcagggccgagaagcacaggtaggggttggccaggggatccgggaaacgggtctcgatacgacggcccttggggttggccacgtgcgggatacggatcgaggccgaacggttgcgggccgagtaggccagcttgaccggggcttcgaagccggggaccagacgcttgtaggagttggtgccggggttggtgatggcgttcagagccttggcgtgcttgatgatgccgccgatgtagtacagcgcgaactcggacaggccagcatagccgtcgccagcgaacaggttcttgccatccttccagacggactggtgaacgtgcatgccgcctccttacgcagggcatccatttattactcaaccgtaaccgattttgccaggttacgcggctggtcctctagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcgtcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgactcgagcgtcgatttttgtgatgctcgtcaggggggcggagcctatggaaaaacgccagcaacgcggcctttttacggttcctggccttttgctggccttttgctcacatgttctttcctgcgttatcaggggattccttaaggtatactttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccgataagctctgataccgctcgccgcagccgaacgaccgagcgcagcgggtcagtgagcgaggaagcggaagagcgcccaatacgcaaaccgcctctccccgcgcgttggccgattcattaatgcagcgcccaaacataacaggaagaaaaatgccccg

SEQ ID NO: 17 - DNA, 10315 bp, synthetic construct
  misc_feature 1..10315: note = glnA upstream homology arm - lac promoter - lacI CDS - tac promoter - glnA downstream homology arm in vector including gentamicin selectable marker and human herpes virus 1 thymidine kinase counter-selectable marker
  source 1..10315: mol_type = other DNA
cccgcgccgcaagcgcaacagcccgccttccagcgattcgctctcggaaatgcccagcctctcgccacgcgagcgcgaagtgctgcgctgctgcctggatggcctctccgtcaccgccatcgccgacaagttcgcccgcagcgtcaagaccatcagcggccagaagcaatcggccttccgcaagctgggcgtgcgcagcgacaacgagctgttcaagatcgaataccagatcaaggacctgtaagaccagcaaggagacagaagatgaaccgcagtgagcctagggtcttgctcgtcgatgattcggcgacgatccgttatgcgatgcagaaccgtttgcaatcgctgggctgccaggtccaggtggcggccgacggcgccgccgcgctggcggcgatccgcgcccagccgcaggggctggtgctgctggattgctttctgccggacctgcccggccacgaggtggcgcgtcaggtgcgcgccctggagcaagaggacccgcagcgtccctacacgcccttgatcggcatttccgccgaggctgatgcggcgcacgtacggctatgcctggacggcggcatggatggcatgctcgacaagcccgtgcagaccgccgacctgcgcaagatgctctcgctctggtgcgatcatgaatgtggcgacgagggcgccgcgctgcccccgcgcgaggaatatgcacgcgacctggcgcgcttgttcctgagcacctcgcagcacgacctggccgccctgcaacaggcccaccgggccggcgaccaggccaccatgggcaagctggcgcaccgcatgaagggcgcggcgctgaccatgcagcgcagcgagatcgtggccctgctggagtgcctggaagaggccatgcaggacgactacgacacccaggacgccatctccctcttgttgagttcactggagaaggtattggccgcaagcgcggcgccgggctaggagcgatcccggccgacgcacagcccggcgctcaggcccccacccacggcaggcccgccttgcaccagccctcgacggtcttgcggtggcccgcggcgtccttgttgccctcgaagccttcgaggatgtcgaagcagtgggtatagccatgctcggtcgccagcttggccgcatgcttggaacgcacgccggaacggcacaggaacagcagcacatcgtcagggttggtgacttcccgcagctgttccaggaattgcggattgggcttgccctcggggtagagattccattgcaccgctagatgctgcgccggcgcgatctggaccacgccgacccagtcgcgctcggcgcgggtgcgtacatcgaccagcttgatgcggctgtcgccttcgagcagggccagtgcttcggccggcgtgactgcgccgcgatagggaagctgtgcatcgacggcgcgttgggtggcggcggagaggatatcttgagtgctcatgctgggttcgctatcaagtgtcaggtataagtgtcaggtatcgttgatcgtggatcgccgtgttgcgccggggcaagccccccaggatatcgcgacaggcaatcgcaccgggagctcagattctaaatccaaaccggcgcacgtccccggcctcgcttgcttcggttgcttcggttgcatgatgtggccgctttggtgcgtacctgctgcggcagcaagcgctgccccagaaaagcgcaggcaccaaaacagggcaattcagcttgtttcttccattagtgcattttaatggtgcatttagataaaaacgcactaaaataatgcatcccctggcggcctctgcaccgcagggatcggcccgcaggccgcgtcctccctaaaatatctcatcctgtgcgcaccctaaaggttggtggcatggaatctgctatcatccgccactgagttcggcgcaagaccgttgtatcaaatgcaaagatccagtcgcagaaaacggctcaacaccccaatttatactacttcctcgttttaggagattcgccatcaaataaaacgaaaggctcagtcgaaagactgggcctttcgttttatctgttgttttggtgatgtcggcgatataggcgccagcaaccgcacctgtggcgccggtgatgccggccacgatgcgtccggcgtagaggatctgctcatgtttgacagcttatcatcgatgcataatgtgcctgtcaaatggacgaagcagggattctgcaaaccctatgctactccctcgagccgtcaattgtctgattcgttaccaattaggatccttatcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagtaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccaccatcgccgcttccactttttcccgcgttttcgcagaaacgtggctggcctggttcaccacgcgggaaacggtcatataagagacaccggcatactctgcgacatcgtataacgttactggtttcatattcaccaccctgaattgactctcttccgggcgctatcatgccataccgcgaaaggttttgcgccagcactagaacttgcggcggatactgtcccatgttgacaattaatcatcggctcgtataatgtgtggaattgtgagcgctcacaattaaagaggagaaattacatatggcaaggacggccgcagaggttttgaagatggtgaaggacaacgaagtcaagtttgtcgacttccgtttcgctgacacccgtggcaaggaacagcacgtttccgtgcctgtttcgcatttcgacatcgacaagttcgaatccggtcatgccttcgacggttcgtcgatcgccggctggaagggtatcgaagcctccgacatgctgctgatcccggacccgaacaccgccaacatcgacccgttcatggaagagacgaccctcttcatgcaatgtgacgtgatcgagccgtccgacggcaagggctatgaccgcgatccgcgctccatcgccaagcgcgctgaagcctacctgaagtcctccggcctgggcgacaccgcctacttcggtccggaacccgaattcttcatcttcgacggtgtgcgttggggcgccgacatgtccggttccttcgtcaagatcgactccgaagaagcttcctggagcaccggcgccaagatcgaaggcggcaactccggccaccgtccgaccgtcaagggcggctacttcccggttcccccggtcgacagcttccaggacatgcgttcggaaatgtccctgatcattgaatccctgggcatccccgtcgaagtgcaccaccacgaagtggctggcgctggccagaacgaactgggcaccaagttctcgaccctggtcgagcgcgctgactggacccagaccatgaagtacgtgatctggaacgtcgcccacacctatggcaagaccgcgaccttcatgcccaagcccctggttggcgacaacggctccggcatgcacgttcaccagtccgtctggaaggatggcaagaacctgttcgctggcgacggctatgctggcctgtccgagttcgcgctgtactacatcggcggcatcatcaagcacgccaaggctctgaacgccatcaccaaccccggcaccaactcctacaagcgtctggtccccggcttcgaagccccggtcaagctggcctactcggcccgcaaccgttcggcctcgatccgtatcccgcacgtggccaaccccaagggccgtcgtatcgagacgcgtttcccggatcccctggccaacccctacctgtgcttctcggccctgctgatggccggtctggatggcgtgcagaacaagatccacccgggcgaagctgcgaccaaggacctgtaccacctcccgccggaagaagacaagctgatcccgaccgtgtgctcctcgctggacgaagcgctggaacacctggacaaggaccgcgagttcctgacccgtggcggcgtgttcaccgacagcatgatcgatgcctacatcgatctgaagatgcaggaagtccagcgcttccgcatgaccacgcacccgatcgaattcgacatgtactactcgctgtaatcgaatccgcatccggacaaaaaggggaagcttgcttcccctttttttatgcgcgccggcaagtcccctgagttgtgtgcattccttgcatcctcatgggagcgcgcccgaaacttgctagtgttcccgcacgaacgactctgctccacgacatgaatccaggctccccgactaccgccatgaagactccgttcccctccctcgaaggactcgacctgctggcctcggcggtgatcatcctcgacgccgagggcggcatcgtctatgccaatgccgccgccgagaacctgctggaaagctccttcaaggtgctctcgcagcagaagctctcggagctcttcctgaacggcaaggagctctcggccctgttctaccaggccatcgaacaccagttcgccgacaagcgcctggacctggtgctggaacgcgccaaccgcgaaccgctgcaggtccacaccatcgtcacgccgctggacaaccccgacatgccggtgctgctggagctgcgcgaaaacgtccagcaattgaagctggaccgcgaagagcgcatcttcgaccagagccaggccaacaaggaactgatccctccttacgcagggcatccatttattactcaaccgtaaccgattttgccaggttacgcggctggtcctctagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagcctttgctggccttttgctcacatgttctttcctgcgttatcaggggattccttaaggtatactttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccgataagctctgataccgctcgccgcagccgaacgaccgagcgcagcgggtcagtgagcgaggaagcggaagagcgcccaatacgcaaaccgcctctccccgcgcgttggccgattcattaatgcagcgcccaaacataacaggaagaaaaatgccccgggaccgaaacaaaaaaagacgcttttcagcgtcttcaataattggtttggtaccgagttatagtccagggcgttactttagttggcttctcccatctcgcgtgcaaacgtgcgcgctaaatcacagatggtcggaatcgagcccggagtggtgacatgggtctgcaccatgccagaggtcagttgtagcagcgcatctctgcaaccagctggcgactgatcgtaatccaggataaatacgtgcattgagcgcagtcttttagccagaacgtccagggcccaggcgaacacgttatacaagtctccattcggggcgagaagttcgggtgcacgaaacaacgtgaacagtgtatcccctatatgagggcgagggcctgcgttggactgaggttccgcaccttgaggagggacggccgtacctgagagctggccccaatcctctcgccagctaccaccacattgaagataacgaactgtattcgccagtaagccatacacgcgacggatcgcagctagcatggccaagtctaacctctcccccggacgctggcgtttagccagacgatcgatatgtcgatcttctggcaatgcgcccagcacaatgttggtcccggggagcgtagggggaataagcgcaacaaatgccagaacagcttgcggagtcatactacccatcaagtaacgcgcggcaggataacacagtaacgcggcaattgggtgacggtcgaagatgagtgtcagtgccggaggtggagcatggctggagcccgcttctcccccaatgtgcggggctagtacagcatcagtgacagcgtacggcatacccatggtgatctgcgccgaggtcatgaccactgcggcatcgccagcagagatttcaccctgatccagtcgatgttgcgtagtataaatattcgctatcgtttcgctggcgcccagaactctccagtaggtcattggttccggcacatacacgatgtcatcgcgggaacccagcgctaccaggagctgggtcgtggtagttttgcccatcccgtgcggtccatcaatataaacccgaagtaaggttggcatcttctgttcggggcggacctccgttgcttcttgctggcgtcgcggacgcaaggctgtacggcggttagaatggcccctactccgcgcagcttggtcaaaggctgaggcatgctgatgaccagggtatgaagccattatttcctccttacgtctaggaacgctagcataatacctaggactgagctagctgtaaaaagatcccctgattccctttgtcaacagcaatggatcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggtgacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcggccaattcgatctagaattccattgagtaagtttttaagcacatcagcttcaaaagcgctctg

SEQ ID NO: 18 - DNA, 8886 bp, synthetic construct
  misc_feature 1..8886: note = nifH upstream homology arm - nifH downstream homology arm in vector including gentamicin selectable marker and human herpes virus 1 thymidine kinase counter-selectable marker
  source 1..8886: mol_type = other DNA
ggaccgaaacaaaaaaagacgcttttcagcgtcttcaataattggtttggtaccgagttatagtccagggcgttactttagttggcttctcccatctcgcgtgcaaacgtgcgcgctaaatcacagatggtcggaatcgagcccggagtggtgacatgggtctgcaccatgccagaggtcagttgtagcagcgcatctctgcaaccagctggcgactgatcgtaatccaggataaatacgtgcattgagcgcagtcttttagccagaacgtccagggcccaggcgaacacgttatacaagtctccattcggggcgagaagttcgggtgcacgaaacaacgtgaacagtgtatcccctatatgagggcgagggcctgcgttggactgaggttccgcaccttgaggagggacggccgtacctgagagctggccccaatcctctcgccagctaccaccacattgaagataacgaactgtattcgccagtaagccatacacgcgacggatcgcagctagcatggccaagtctaacctctcccccggacgctggcgtttagccagacgatcgatatgtcgatcttctggcaatgcgcccagcacaatgttggtcccggggagcgtagggggaataagcgcaacaaatgccagaacagcttgcggagtcatactacccatcaagtaacgcgcggcaggataacacagtaacgcggcaattgggtgacggtcgaagatgagtgtcagtgccggaggtggagcatggctggagcccgcttctcccccaatgtgcggggctagtacagcatcagtgacagcgtacggcatacccatggtgatctgcgccgaggtcatgaccactgcggcatcgccagcagagatttcaccctgatccagtcgatgttgcgtagtataaatattcgctatcgtttcgctggcgcccagaactctccagtaggtcattggttccggcacatacacgatgtcatcgcgggaacccagcgctaccaggagctgggtcgtggtagttttgcccatcccgtgcggtccatcaatataaacccgaagtaaggttggcatcttctgttcggggcggacctccgttgcttcttgctggcgtcgcggacgcaaggctgtacggcggttagaatggcccctactccgcgcagcttggtcaaaggctgaggcatgctgatgaccagggtatgaagccattatttcctccttacgtctaggaacgctagcataatacctaggactgagctagctgtaaaaagatcccctgattccctttgtcaacagcaatggatcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcggccaattcgatctagaattccattgagtaagtttttaagcacatcagcttcaaaagcgctctgccgtgcacgaacggcagcgtgttctggaagccgacggcggcatacaccgcccccagcggctggcaagccttggccggattgatggtcagcgcctcgcgggcgaagttcttttgctggtattccttggtcttggtccattcggccacgtcctgcaccttgtccagcggaacggcaaactcgaactcttctttcttgcgggcgaacagttcgcggtattccggctggcggaacagattctcgtggtccagaacgctatcggcgttttgcggcatgatgataccccttcaattcaactgatcaaatgatccaggctcgggtcggatggacgatgagcgatcgacacagtcctgcgcccatccatcccgaatcgattcagctcaggccttccacggagccttggccatgccccacaccgggctgttgatggccatgtccatgtcgcgggcgaagatggcgaagccgtcatagccgtgatacgggcccgagtaatcccaggagtgcatctggcggaacggcacgcccatcttctggaagacgtatttctccttgatcccggagccaaccagatcgggctcgatcttctcgacgaacttctcgaattcatagctggtcacgtcgtcgtagatcagcgtgccgtcttccacgtagtgggtggtgcgctggtagtcgtcgttgtggccgaattcatatccggtaccgaccaccttcatgcccaggtcaccataggcatcgatcacgtggcgcgggcgcaggccaccgacgaacagcatcacggtcttgccttccaggcgcgggcggtacttggcgatcacggcatcggtgagggccttgtacttggcgatgacacgctcggcgccttccttgatcttgtcgtcgaagtggctggcaatctggcgcagcgacgcctcgatctggctggggccgaagaagttgtactcgacccagggaatgccgaacttctcttccatgtggcgcgagatgtagttcatcgagcgatagcagtgcagcacgttgagcttggccttgggggtgttttccatttcggcgagggtgccgtcgccggaccactgggcgatcacgcgcaggccgatttcttccagcaggatgcgcgaggaccaggcgtcgccgccgatgttgtagtcgccgatgatggccacgtcatacggagtcgccacaaactggttcttgtccgggtccatcttgtccagcacccattccttgatggcgtcgttggccacgtggtggcccagcgactgcgacacgccacggaagccttcgcagcgcaccggcacgatggtatggccctcgtattgcttggactttttcttggagaccgcttcgatgtcgtcaccgatgagaccgatcgggcattcggattgcaccgaaatacccttgttgagcgggaacagttcctggatctcgtcgacgatcttttccagcttcttatcgccgccgaagacgatgtccttttcctggaaatcggacgtgaactgcatggtcacgaaactgtcgatgccggtcttgccaatgtaatagttgcggcgcgagccccaggagtactggccacagccgaccggaccatggctgatgtggatcatgtccttgatcggcccccagaccacgcccttggaaccggcataggcgcagccgcgaatggtcatcacgccggggatcgacttgatgttggatttgacgttgcaatccgacttgccctcttcctgggtggtcaggtgcttggcgcgacgcttggcggtcttgtccggataggccttgaggacctcattgatcaattccgtattgcgtgccgtggtttcttcaactgtgaggctcatgggccctccgataaaaattagtcgcgggggacatacccggtcgtatgccagtccggtaaacaaacctgcgctaccctttttcttgcggtcccggctgccttcgcttctgaacacagaggcgcgagcggccaggaccttgcagcatcaacagcaggcagacgacgatgcttgattctcctagaaataagcggtgtagctctggtgtaagaaattaaggtttcgttcaaagcagttttgtcatcacacttgcagccgttggggcgccgcttcgaaacttttattggtcttcgtctcaagtcacgcattcggcaagtcatcgtcggtgcacacctctctttgcaaatgccgtgccaccatcaaaaaacgattccaagccattgatttaattggtgaaaacagcatttcgcgcagcctctcgacccacttctttctggtgttacaaaccgcacaaacggcacctggtctgtagggaacgatacaagcccgcatccccgacacaaccgtgtacccaaccaccctgttgcacccctctcggcccgctggcccgcagccacgggccttgtcacgccctgctgaagacaagcagaggcggctcgctgataacgggaacgatgtttgctatcgggatgaatggcagggcaaagaaggctgcgccaagcggcccggtcagtgatccactcacgcaaggagcaaaccatgcaaatcggtgtagacagccagcgcgcggtcgacaacaaacgggtcttcatcgtcgactgtgatgaagtcagcggcttggcgctgcagttcatgctcgccgacgagtgcgaagcgcatctgctggcggatgtggacaatgccttgcgcaagggccgggattggccgcccgacctgctcttgctgggccagggcaatctcgtcaaggagggcatggaggtgctggcccaattgcgccagcaacatccggccttgaagatcgtagtggtgtgcgacagcgccaatgaccgccatgttcgccaagccctggcgcaaggggccaactccacgctgctgcgcccgcttacgctggagagcgtgcgccgcaaggtcgatgcccaactgggccgacgcacgcccttgggcctgggcatcgcggtgacggtgatgtaagaccatgtgcgcgactcgcatccccttgaagctgccgcatcgggctaccccgatgccgggcgcatcgaagtctgcgccgctggccaatccgcattaccagcagatcggcggcgagccggtcgtgcggcaactggtcgaccgtttctacgccctgatggaccagttgccccaggcggccgtggtacgcgccatgcatccgggcgacctgaccccggccaagcagcgcctgttcatgttcctctccggttggctgggcggcccgccgctgtatgccgaggccttcggccatccgcgcctgcgtcacgcacacgcgggctttgccgtcgatgctgccgcacgtgatgcctggatggcctgcatgaaccaggcactacaagaactggtacccgacgaggtgttgcggcagcaactgggtgccgcatttttcaagactgccgattttctgtgcaaccaataaggaaacttcatgagcactctcgcaaatacccccctggccgatctcgacaatgatggccgtctgctgagcccggtcttcaagggcccgaccaaacgcaatggacgcgtcgccttccgtggcgaaatcgcgctcaaataccaggccagcctggccgatgaaaagcgccccccggaaatcaagctcgaccaggtgatgacggtggccgacgtgggcgccaccaccattcctttcctggccggcatggcgccttcgctggaacatatcaagctgctgtgcgaagtgctgggggacaagctggctgccgatggcaaatatttcttctttgcctgcaacctggacatttcgctgcggttccagattccttatggcggcgccaccttctacgtgatgccgctggacgaatcgacggtctataacgagctgctggaattgttccgcatcgagcgtggcgatctcaagaaactcgacaccgccgaaaagattctggcggtggccgacggtgccctgcgcttcaagggcaagttcgaggagatcagcttccagcgcgggctggaggtgatggggccgatcaagatcaaggaaacctccttacgcagggcatccatttattactcaaccgtaaccgattttgccaggttacgcggctggtcctctagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcgtcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgactcgagcgtcgatttttgtgatgctcgtcaggggggcggagcctatggaaaaacgccagcaacgcggcctttttacggttcctggccttttgctggccttttgctcacatgttctttcctgcgttatcaggggattccttaaggtatactttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccgataagctctgataccgctcgccgcagccgaacgaccgagcgcagcgggtcagtgagcgaggaagcggaagagcgcccaatacgcaaaccgcctctccccgcgcgttggccgattcattaatgcagcgcccaaacataacaggaagaaaaatgccccg

SEQ ID NO: 19 - DNA, 8885 bp, synthetic construct
  misc_feature 1..8885: note = nifA upstream homology arm - nifA downstream homology arm in vector including gentamicin selectable marker and human herpes virus 1 thymidine kinase counter-selectable marker
  source 1..8885: mol_type = other DNA
ggaccgaaacaaaaaaagacgcttttcagcgtcttcaataattggtttggtaccgagttatagtccagggcgttactttagttggcttctcccatctcgcgtgcaaacgtgcgcgctaaatcacagatggtcggaatcgagcccggagtggtgacatgggtctgcaccatgccagaggtcagttgtagcagcgcatctctgcaaccagctggcgactgatcgtaatccaggataaatacgtgcattgagcgcagtcttttagccagaacgtccagggcccaggcgaacacgttatacaagtctccattcggggcgagaagttcgggtgcacgaaacaacgtgaacagtgtatcccctatatgagggcgagggcctgcgttggactgaggttccgcaccttgaggagggacggccgtacctgagagctggccccaatcctctcgccagctaccaccacattgaagataacgaactgtattcgccagtaagccatacacgcgacggatcgcagctagcatggccaagtctaacctctcccccggacgctggcgtttagccagacgatcgatatgtcgatcttctggcaatgcgcccagcacaatgttggtcccggggagcgtagggggaataagcgcaacaaatgccagaacagcttgcggagtcatactacccatcaagtaacgcgcggcaggataacacagtaacgcggcaattgggtgacggtcgaagatgagtgtcagtgccggaggtggagcatggctggagcccgcttctcccccaatgtgcggggctagtacagcatcagtgacagcgtacggcatacccatggtgatctgcgccgaggtcatgaccactgcggcatcgccagcagagatttcaccctgatccagtcgatgttgcgtagtataaatattcgctatcgtttcgctggcgcccagaactctccagtaggtcattggttccggcacatacacgatgtcatcgcgggaacccagcgctaccaggagctgggtcgtggtagttttgcccatcccgtgcggtccatcaatataaacccgaagtaaggttggcatcttctgttcggggcggacctccgttgcttcttgctggcgtcgcggacgcaaggctgtacggcggttagaatggcccctactccgcgcagcttggtcaaaggctgaggcatgctgatgaccagggtatgaagccattatttcctccttacgtctaggaacgctagcataatacctaggactgagctagctgtaaaaagatcccctgattccctttgtcaacagcaatggatcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcggccaattcgatctagaattccattgagtaagtttttaagcacatcagcttcaaaagcgctcttttttctccttgcgtgaaactgattggctgcttacgctgcggcggacgtcgactgcaccgaggtatacgccccctggcggatatcggcgtcgcctcgtggccgatgctcgatctgtccgctgcgcacgcgttccaggtaatccatgaagtaggcgatgacggattgctcgatgaattcatgggcataccggtccaccggctcgatgcccaccttctgcagatcatccttgggacagccgccgatcttggccaccagcactgcatggcagtcgttgatggcgcggatgacggtctccagtgcatcctcctcgccatagccgccctgacagtactggtccacgcgccggtgtccgacgaacttgctgccggcgctgctgacttcatagatctggaattcgctgacgtgtccgaagtgctcgttgacgcgtccctgccccttggtcgccaccgccaccagcaaggacaggccgctctggtctgccatctgggccagctcctgctgctcggcggccttggcggccgacttggcctggcgttcctgttccaccagctcctggtaagccttgcgggtggcgccgtcataggccacttccatggcctcgatcttttcggtggtgaattcggcgctgcggtcttcacccagcaggcccaccgcatcggcgcggcactggcggcagtggcgcatcatgttcatctcgccttcacaggcatcctgcaaggccttgagctcctgcgcgctggggccgcgctggccgttcaggccaaacacggtgccatgctcgggcgctgaaatgagcggcatgatgttgtgcaggaaggcaccgcgtgacttgacggcgcgattgacttccaccagatgctggtcattgatgcccgggatcatcaccgagttgaccttgcacaggatgccgcgcgccgtgagcatctccagtcccagcatctgccgttcgtgcaggatgcgggccgcgtcgataccggtccagcgcttgttctggtaatagatccagggatagatgtgctgtccgatctcgggatcgaccatgttgatggtgatggtgacgtgatcgacgttaaaggcagcaatggtatcgatgtggtcaggcaaggccaggccattggtcgacaggcacagcttgatatcgggcgcggtctgcgaaatcagctcgaaggtcttgaaggtcttggccggattggccagcgggtcgccaggaccggcaatgcccagcaccgtcatctgcggaatggtcgaggccaccgcaaagaccttcttggccgcttgttcgggcgtgagcttttcgctgaccacgcctggacgcgactcgttggcgcaatcgtatttgcggttgcagtagttgcactggatgttacaggccggtgccaccgccacgtgcatacgcgcatagtggtgatgagcctcttcgctgtagcagggatggttcttgaccttttcccaaacctcgggcgccatgtcggcgggaccatcggatgaaccgcaactggctttgccgccttcactgctggtgccacagcctttgtgttcggctaccttgtccagcaaggtgcctagcgatttgatgtcctggataccgacgtattgtgtgggctgcatggatagtccctttcaggaaagtgatgcctgggccttctcctgaggacatagccaaaaccgtgccagacaaaataatcaatcaattcaatgacatagcggagaattgcgtcgtacggcgatgtgaggatagcgacaatcggcgatacattgtgggtggcgcttgtcatgaacacgacaatgcagcacgggccggcgcctgcaattgagcgcgccgcaatgagcctgacgcagtggagaaggagaaggagaatgcgaggacgtgccagccggggaatccatcaatacaaccggctggcgcgacgccgaagagaccgcgcgcgcagggcgaggacggcaatgagaaaaagactttggatctggcagcgtattacgtcacgctcgcagatgagttctgactctcctaacgtgaatactggaaccccatgcgctgcctgagagcgctagaacccgccttgctactctgtcgtgagatccgagatcctgaaaaacggcagaaaaagcgccgtgtaattaccgaatagttaccttgctggtcaagcaaacttcatgcccaagcagatgcatgaatcgatctcgccaagcaaccccacacaatgccggctttcctcgatgtcgccttggcgggttggcgacaacggtgtcgtctttgtcggatttacgacaatcatcgccactggcgcagcaatcagaaatcgtcgctatcccattgatttaaaagaaaaatattgtatggcatggttttggctattcatacctgcatcggccgctcaccggccaagcgaaacgacaaagcagagcaagccggtagcccagccagcgttatgtagcgagatatacagacctccaacatccaccatcgcccactatgaatgtgccttcgacttccgaacccggtgcaccacgtgcgtgctgggcgccctgctcccaggcatgttatctgaccctgaactggccggtcgccggcccggcgatggcgcgcggcacgcaacaggtgatatgtgagccgccacaattacctcaaccggcgcaccggttgctggcgctgtgtgatgaatccggcttgcgcaaactggtgctgcatcatatgcggcgcctgcgccattccccgctgttcgcacgttcgcacaattgttttgattgcgtcagctcgcgcatcgccgacttcgtggtggagtcatgcggcggtccgctctactacagccaacgccatgcccatctgcaagcgggcgcaggcttgccgttgctattggacgaagctgggcgcgaactgtggctggtacagctttggcatacctttgacgatatcggtttcccgcccgcgctgcgggccgacttctgggcctgggccgaacccttgtcgattcatctgctggtacgtcatgcgcgcgtcgaaccgccgcggcgctacccctatgagctggtacgcagctggttccactcgcccgccaccgacatgctgccgccgatagcggatctgatccggccatcgggaagatccgaatcatgacctgcgccatccggcatcgcatccccgcatctgtttcttgatgcggccggtctgagcgatgtccaactctttttcccgtgaacctgccatgccggattacccgcaacgctacggcgtcttcctcaatgtcgaaaatccccatggcaacgcacgcgatgcgctgttgcagaccgtcgatcatgccgtcgtggcagaggccctgggctatcacgatgtctgggtcgccgagcaccattacagcagcttcgccatcggcagcgccttgatggtgctgctggcccagatcgccgcacgcacttcgcgcatccgcctcggtacgggcgcttcgttgctggcgctcaatgatcccttgcgggttgccgaggacgtcgccacgctggacctgctcagcaacggccggatggaattcggcgtggcacgcggcggcccgtttcccgagcaatatcgtcacgccggattgagcggtgccgatcaggcgcgcgcacgcatgattgaagcactggacgtcatccagcgcgcatggcaaggcgtgccattcagtaccgagggacgattttttcactacgacagagtgcagttgcaaccgcagccgctgcaacgtcccatcccggtatggctggccagcatgagcgaagattcgcttgatctggccgcacgccaggattacggattgatggccacaccctccgccgatcttgagcaggtgctcacgcgggtggctgcacagcgtgcgcagcgcggtcacttcccgtttgccatcgcccgctttttccattgcgaacccgaggcgcacaaggcgcttgcccatggtctggcaggtgtgcgtgcgtatccgcgcatgatgcaagtgcaattcgcgcccggtggcaccccgcccatgttcccctccttacgcagggcatccatttattactcaaccgtaaccgattttgccaggttacgcggctggtcctctagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcgtcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgactcgagcgtcgatttttgtgatgctcgtcaggggggcggagcctatggaaaaacgccagcaacgcggcctttttacggttcctggccttttgctggccttttgctcacatgttctttcctgcgttatcaggggattccttaaggtatactttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccgataagctctgataccgctcgccgcagccgaacgaccgagcgcagcgggtcagtgagcgaggaagcggaagagcgcccaatacgcaaaccgcctctccccgcgcgttggccgattcattaatgcagcgcccaaacataacaggaagaaaaatgccccg